(19) United States
(12) Patent Application Publication
(10) Pub. No.: US 2004/0229299 A1
(43) Pub. Date: Nov. 18, 2004

(54) Intracellular complexes as biomarkers

(76) Inventors: M. Youssouf Badal, Sunnyvale, CA (US); Xueguang Jin, Santa Clara, CA (US); Hossein Salimi-Moosavi, Sunnyvale, CA (US); Singh Sharat, Los Altos, CA (US)

Correspondence Address: ACLARA BIOSCIENCES, INC., 1288 PEAR AVENUE, MOUNTAIN VIEW, CA, 94043, US

(21) Appl. No.: 10/814,686

(22) Filed: Mar. 30, 2004

Related U.S. Application Data
(63) Continuation-in-part of application No. 10/154,042, filed on May 21, 2002.
(63) Continuation-in-part of application No. 10/623,057, filed on Jul. 17, 2003.
(60) Provisional application No. 60/459,888, filed on Apr. 1, 2003.
(60) Provisional application No. 60/494,482, filed on Aug. 11, 2003.
(60) Provisional application No. 60/508,034, filed on Oct. 1, 2003.
(60) Provisional application No. 60/512,941, filed on Oct. 20, 2003.
(60) Provisional application No. 60/523,258, filed on Nov. 18, 2003.

(51) Int. Cl. G01N 33/574

(52) U.S. Cl. 435/7.23

(57) ABSTRACT

The invention provides a method for determining a disease status of a patient by measuring expression levels of selected intracellular complexes. In one aspect of the invention, the activation status of apoptotic pathways in a patient sample is determined by measuring relative amounts of protein-protein complexes that are characteristic of the apoptotic pathways. In particular, the invention provides a method of determining the activation status of the mitochondrial apoptotic pathway by simultaneously measuring relative amounts of complexes between 14-3-3 proteins and BAD proteins on the one hand and complexes of Bcl-2 proteins and BAD proteins on the other hand. Preferably, methods of the invention are implemented by using sets of binding compounds having releasable molecular tags that are specific for multiple components of one or more complexes formed within apoptotic pathways. After binding, molecular tags are released and separated from the assay mixture for analysis.

This is a continuation-in-part of U.S. patent applications Ser. No. 10/154,042 filed 21 May 2002 and Ser. No. 10/623,057 filed 17 Jul. 2003; priority is further claimed under U.S. provisional applications Ser. No. 60/459,888 filed 1 Apr. 2003; Ser. No. 60/494,482 filed 11 Aug. 2003; Ser. No. 60/508,034 filed 1 Oct. 2003; Ser. No. 60/512,941 filed 20 Oct. 2003; and Ser. No. 60/523,258 filed 18 Nov. 2003, all of the above of which are incorporated by reference in their entirety.

### FIELD OF THE INVENTION

The present invention relates generally to biomarkers, and more particularly, to the use of intracellular molecular complexes as biomarkers.

### BACKGROUND OF THE INVENTION

A biomarker is a characteristic that is objectively measured and evaluated as an indicator of normal biological processes, pathogenic processes, or pharmacological responsees to a therapeutic intervention, Atkinson et al, Clin. Pharmacol. Ther., 69: 89-95 (2001). Biomarkers vary widely in nature, ease of measurement, and correlation with physiological states of interest, e.g. Frank et al, Nature Reviews Drug Discovery, 2: 566-580 (2003). It is believed that the development of new validated biomarkers will lead both to significant reductions in healthcare and drug development costs and to significant improvements in treatment for a wide variety of diseases and conditions. Thus, a great deal of effort has been directed to using new technologies to find new classes of biomarkers, e.g. Petricoin et al, Nature Reviews Drug Discovery, 1: 683-695 (2002).

The formation and disassociation of molecular complexes is a pervasive biological phenomena that is crucial to regulatory processes in living organisms. In particular, signaling pathways between the extracellular environment and the nucleus of a cell involve the formation of many molecular complexes in which multiple proteins are assembled to directly or indirectly induce molecular events, such as phosphorylation or dephosphorylation, which are steps in the signaling process, Gomperts et al, Signal Transduction (Academic Press, New York, 2002). Such pathways and their components have been the subject of intense investigation because of the role aberrant pathway behavior plays in many disease conditions, especially cancer, e.g. McCormick, Trends in Cell Biology, 9: 53-56 (1999); Blume-Jensen and Hunter, Nature, 411: 355-365 (2001); Nicholson et al, Cellular Signalling, 14: 381-395 (2002); and the like. For example, it has been observed that many cancers are associated with an accumulation of mutations or other genetic alterations that affect components of signaling pathways, e.g. by over expression, particularly those pathways involved with cell proliferation, cell motility, differentiation, and cell death, e.g. Blume-Jensen and Hunter (cited above); Evan and Vousden, Nature, 411: 342-348 (2001).

Signaling pathways have been difficult to study not only because of their complexity and interconnectedness, but also because of the disruptive procedures required for analysis of intracellular complexes, e.g. Weng et al, Science, 284: 92-96 (1999); Machida et al, Molecular & Cellular Proteomics, 2.4: 215-233 (2003); Price et al, Methods in Molecular Biology, 218: 255-267 (2003). A wide variety of techniques have been used to study cellular protein-protein interactions and complexes, including immunoprecipitation, chemical cross-linking, yeast two-hybrid systems, tagged fusion proteins, bioluminescence resonance energy transfer (BRET), fluorescence resonance energy transfer (FRET), mass spectroscopy, and the like, e.g. Golemis, editor, Protein-Protein Interactions (Cold Spring Harbor Laboratory Press, New York, 2002); Price et al (cited above); Sorkin et al, Curr. Biol., 10: 1395-1398 (2000); McVey et al, J. Biol. Chem., 17: 14092-14099 (2001); Salim et al, J. Biol. Chem., 277: 15482-15485 (2002); Angers et al, Proc. Natl. Acad. Sci., 97: 3684-3689 (2000); Jones et al, Proteomics, 2: 76-84 (2002); and Petricoin III, et al, The Lancet, 359: 572-577 (2002). Unfortunately, such techniques are difficult to apply, generally lack sufficient sensitivity to provide an accurate picture of the state of a signaling pathway, and/or cannot measure multiple components or interacting components that are crucial for pathway activation. Consequently, measurements made by such techniques have not led to useful biological indicators based on complex formation.

In view of the above, the availability of a new class of biomarkers based on the presence, absence, and/or profile or ratios of intracellular complexes involved with key intracellular processes, such as signal transduction, would advance the medical field by providing a new tool for diagnosis, prognosis, patient stratification, and drug development.

### SUMMARY OF THE INVENTION

The invention is directed to biomarkers comprising intracellular molecular complexes in patient cell or tissue samples, particularly samples preserved by conventional procedures, such as freezing or fixation. In one aspect, the invention includes the correlation of amounts of one or more intracellular molecular complexes to the status of a disease or healthful condition. In particular, the invention provides biomarkers for indicating the status of signaling pathways involved with a disease or healthful condition, wherein such biomarkers are the amounts of signaling complexes.

In one aspect, the invention permits the determination of a disease status of a patient suffering from a disease characterized by aberrant expression of one or more intracellular molecular complexes by the following steps: (i) measuring an amount of each of one or more intracellular molecular complexes in a patient sample; (ii) comparing each such amount to its corresponding amount in a reference sample; and (iii) correlating differences in the amounts from the patient sample and the respective corresponding amounts from the reference sample to the disease status the patient. A patient sample may be fixed or frozen; however, preferably, a patient sample is fixed using conventional protocols.

In one aspect, the invention provides a method of determining a status of a cancer in a patient by simultaneously determining relative amounts of signaling pathway complexes. In one embodiment, such complexes are measured using at least two reagents, referred to herein as “reagent pairs,” that are specific for different members of each complex: one member, referred to herein as a cleaving probe, has a cleavage-inducing moiety that may be induced to cleave susceptible bonds within its immediate proximity; and the other member, referred to herein as a binding compound, has one or more molecular tags attach by linkages that are cleavable by the cleavage-inducing moiety. In accordance with the embodiment, whenever such different members form a complex, the cleavable linkages are brought within the effective cleaving proximity of the cleavage-inducing moiety so that molecular tags can be released. The molecular tags are then separated from the reaction mixture and quantified to provide a measure of complex formation.

In another aspect of the invention, amounts of intracellular molecular complexes in a patient sample may be expressed as a ratiometric measure of complexed and uncomplexed components, or as profiles of relative amounts of a plurality of such complexes. That is, the amount of an intracellular molecular complex is given as a ratio of a measure of one component present in the complex to a measure of the total amount of the other component of the complex, whether it is present in the complex or in a free or monomeric state. In one embodiment, typical measures include peak height or peak area of peaks in an electropherogram that are correlated to particular molecular tags. In another embodiment, the invention correlates ratiometric measurements of one or more protein-protein complexes with a disease status.

In a particular aspect, the invention provides a method of determining from measurements on patient samples, especially fixed samples, the apoptotic status of a patient suffering from a disease, such as a cancer, characterized by aberrant apoptosis, wherein such measurement are of the types and/or amounts of intracellular molecular complexes forming elements of apoptosis signal transduction pathways. Such intracellular complexes include, but are not limited to, one or more of 14-3-3//BAD, BID//BAX, BAX//BAX, Bcl-X_{L}//BAD, Bcl-2//BAD, 14-3-3//BID, BID//BAK, BAX//Bcl-2, Bcl-X_{L}//BIK, Bcl-2//BIK, NF-kB//I-kB, BID//Bcl-2, Bcl-X_{L}//BID, Bcl-2//BID, FADD//caspase-9, BID//Bcl-X_{L}, Bcl-X_{L}//Hrk, Bcl-2//Hrk, TRADD//caspase-9, BID//A1/Bfl-1, Bcl-X_{L}//BIM, Bcl-2//BIM, Apaf-1//caspase-9, Bcl-X_{L}//Noxa, Bcl-2//Noxa, Bcl-X_{L}//Bmf, Bcl-2//Bmf, Bcl-X_{L}//Puma, Bcl-2//Puma, Bcl-X_{L}//Bcl-G, Bcl-2//Bcl-G, Bcl-X_{L}//NIP3, Bcl-2//NIP3, Bcl-X_{L}//Nix, and Bcl-2//Nix. In another aspect, such intracellular complexes include, but are not limited to, 14-3-3//BAD, Bcl-2//BAD, 14-3-3//BID, BAX//Bcl-2, Bcl-2//BIK, BID//Bcl-2, Bcl-2//BID, Bcl-2//Hrk, Bcl-2//BIM, Bcl-2//Noxa, Bcl-2//Bmf, Bcl-2//Puma, Bcl-2//Bcl-G, Bcl-2//NIP3, and Bcl-2//Nix. In another aspect, such intracellular complexes include, but are not limited to, NF-kB//I-kB. In still another aspect, such intracellular complexes include the measurement of NF-kB//I-kB complexes to determine a disease status of a patient suffering from a cancer or an inflammatory conditions, such as rheumatoid arthritis, inflammatory bowel disease, multiple sclerosis, or asthma. Preferably, the above measurements are ratiometric measurements that provide values for ratios of free and complex-bound proteins.

In a particular aspect, the invention provides a method of determining from measurements on patient samples, especially fixed samples, a disease status of a patient suffering from a disease, such as a cancer, characterized by aberrant signal transduction pathway activation, wherein such measurement are of the types and/or amounts of intracellular molecular complexes forming elements of selected signal transduction pathways. Such intracellular complexes include, but are not limited to, one or more of Her1//Shc, Grb2//Sos, Her1//Grb7, Her1//RasGAP, Grb2//Shc, Her2//Shc, Her3//PI3K, Her3//Shc, Her3//Grb7, YAP//Her4, IGF-1R//PI3K, IGF-1 R//Shc, IGFR//IRS1, VEGFR//Shc, VEGFR//PI3K, VEGFR//Src, VEGFR//FRS2, PDGFRa//Crk, PDGFR//Grb2, PDGFR//Grb7, PDGFR//Nck; PDGFR//Shc, DGFR//STAT5, PDGFRa//Crk, PDGFRb//GAP, PDGFR//Grb2, PDGFR//Grb7, PDGFR//Nck; PDGFR//Shc, PDGFR//Shp2, PDGFR//RasGAP, PDGFR//STAT5, PDGFRb//GAP, PDGFR//Grb2, PDGFR//Grb7, PDGFR//Nck, PDGFR//Shc, PDGFR//Shp2, PDGFR//RasGAP, PDGFR//STAT5, Kit//Shp-1, Kit//PI3K, Kit//Grb2, Kit//CRKL, FGFR//PLCg1, FGFR//Crk, FGFR//FRS2, GFR//Shp2, FGFR//Shb, Trk//p75NTR, and Trk//PI3K. In another aspect, such intracellular complexes include, but are not limited to, Her1//Shc, Grb2//Sos, Her1//Grb7, Her1//RasGAP, Grb2//Shc, Her2//Shc, Her3//PI3K, Her3//Shc, and Her3//Grb7. In another aspect, such intracellular complexes include, but are not limited to, IGF-IR//PI3K, IGF-IR//Shc, and IGFR//IRS 1. In another aspect, such intracellular complexes include, but are not limited to, VEGFR//Shc, VEGFR//PI3K, VEGFR//Src, and VEGFR//FRS2. In another aspect, such intracellular complexes include, but are not limited to, PDGFRa//Crk, PDGFR//Grb2, PDGFR//Grb7, PDGFR//Nck; PDGFR//Shc, DGFR//STAT5, PDGFRa//Crk, PDGFRb//GAP, PDGFR//Grb2, PDGFR//Grb7, PDGFR//Nck; PDGFR//Shc, PDGFR//Shp2, PDGFR//RasGAP, PDGFR//STAT5, PDGFRb//GAP, PDGFR//Grb2, PDGFR//Grb7, PDGFR//Nck, PDGFR//Shc, PDGFR//Shp2, PDGFR//RasGAP, and PDGFR//STAT5.

In another aspect, the invention provides a method of determining a status of a cancer in a patient by simultaneously determining relative amounts of apoptotic pathway complexes comprising a complex of a Bcl-2 protein and a BH3-only protein and a complex comprising a 14-3-3 protein and a BAD protein. In one embodiment, such complexes are measured using at least two reagents that are specific for different members of each complex: one member, referred to herein as a cleaving probe, has a cleavage-inducing moiety that may be induced to cleave susceptible bonds within its immediate proximity; and the other member, referred to herein as a binding compound, has one or more molecular tags attach by linkages that are cleavable by the cleavage-inducing moiety. In accordance with the embodiment, whenever such different members form a complex, the cleavable linkages are brought within the effective cleaving proximity of the cleavage-inducing moiety so that molecular tags can be released. The molecular tags are then separated from the reaction mixture and quantified to provide a measure of complex formation.

The present invention provides biomarkers comprising measures of the amounts of intracellular molecular complexes in patient samples. In particular, profiles of intracellular molecular complex populations may be correlated to disease status of a patient, and in some embodiments, to prognosis, efficacy of drugs acting on the measured complexes, and likelihood of patient responsiveness to therapy. In accordance with the invention, short comings in the art are overcome by enabling the direct measurement of intracellular molecular complexes in patient samples without the need to culture or otherwise process the cell or tissue samples by methodologies, such as xenografting, that increase cost and labor as well as introducing sources of noise and potential artifacts into the final assay readouts. The present invention also provides a surrogate measurement for intracellular receptor and/or complex phosphorylation, or other post-translational modifications, that are easily destroyed in sample preparation procedures. Such surrogate measurements are based on the measurement of complexes, such as PI3K or SHC-receptor complexes, and the like, that depend on the above modifications for their formation and that are less affected by, and more stable under, sample preparation procedures.

### BRIEF DESCRIPTION OF THE DRAWINGS

FIGS. 1A-1E illustrate diagrammatically several embodiments of the method of the invention for measuring the presence of intracellular complexes.

FIGS. 1F-1G illustrate diagrammatically the use of releasable molecular tags to measure cell surface receptor complexes in fixed tissue specimens.

FIG. 1H illustrates the ratiometric measurement of two complexes of dimers.

FIGS. 2A-2E illustrate diagrammatically methods for attaching molecular tags to antibodies.

FIGS. 3A-3F illustrate oxidation-labile linkages and their respective cleavage reactions mediated by singlet oxygen.

FIGS. 4A-4J show the structures of tags that have been designed and synthesized.

FIGS. 5A-5D illustrate the chemistries of synthesis of the tag moieties illustrated in FIG. 6.

FIGS. 6A-6C diagrammatically illustrate a microfluidics device for implementing a step of electrophoretically separating molecular tags.

FIGS. 7A-7D illustrate the assay design and experimental results for detecting a PI3 kinase-Her3 receptor activation complex.

FIGS. 8A-8D illustrate the assay design and experimental results for detecting a Shc/Her3 receptor-adaptor complex.

FIGS. 9A-9C illustrate an embodiment of the invention for simultaneously measuring BAD//14-3-3 and BAD//Bcl-2 complexes.

FIG. 10 shows data for a correlation between expression of Her2-Her3 heterodimers and PI3K//Her3 complexes in tumor cells.

### DEFINITIONS

“14-3-3 protein” means a human protein capable of forming a stable complex with a human BAD phosphorylated at Ser-112 and/or Ser-155, such protein having an amino acid sequence substantially identical to that described under NCBI accession number AAH56867 or in Strausberg et al, Proc. Natl. Acad. Sci., 99: 16899-16903 (2002). In one aspect, a 14-3-3 protein hereunder is at least eighty percent identical, and more preferably ninety percent identical, to the amino acid described under NCBI accession number AAH56867 or in Strausberg et al, Proc. Natl. Acad. Sci., 99: 16899-16903 (2002).

“Akt protein” means a human protein that is a member of the set of PKBa/Akt1, PKBb/Akt2, PKBg/Akt3, PKBg-1, and proteins having substantially identical amino acid sequences thereof, and that has protein kinase activity whenever phoshorylated by a PI3K protein. In one aspect, an Akt protein has kinase activity whenever either or both of a tyrosine at a location number from 305 to 310 is phosphorylated and a serine at location number from 470 to 475 is phosphorylated. Akt proteins are described under various NCBI accession numbers, including NP_{—}005154, and in Nicholson et al, Cellular Signalling, 14: 381-395 (2002); Kandel et al, Exp. Cell. Res., 253: 210-229 (1999); and like references, which are incorporated herein by references.

“Antibody” means an immunoglobulin that specifically binds to, and is thereby defined as complementary with, a particular spatial and polar organization of another molecule. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)2, Fab′, and the like. In addition, aggregates, polymers, and conjugates of immunoglobulins or their fragments can be used where appropriate so long as binding affinity for a particular polypeptide is maintained. Guidance in the production and selection of antibodies for use in immunoassays, including such assays employing releasable molecular tag (as described below) can be found in readily available texts and manuals, e.g. Harlow and Lane, Antibodies: A Laboratory Manual (Cold Spring Harbor Laboratory Press, New York, 1988); Howard and Bethell, Basic Methods in Antibody Production and Characterization (CRC Press, 2001); Wild, editor, The Immunoassay Handbook (Stockton Press, New York, 1994), and the like.

“Antibody binding composition” means a molecule or a complex of molecules that comprises one or more antibodies, or fragments thereof, and derives its binding specificity from such antibody or antibody fragment. Antibody binding compositions include, but are not limited to, (i) antibody pairs in which a first antibody binds specifically to a target molecule and a second antibody binds specifically to a constant region of the first antibody; a biotinylated antibody that binds specifically to a target molecule and a streptavidin protein, which protein is derivatized with moieties such as molecular tags or photosensitizers, or the like, via a biotin moiety; (ii) antibodies specific for a target molecule and conjugated to a polymer, such as dextran, which, in turn, is derivatized with moieties such as molecular tags or photosensitizers, either directly by covalent bonds or indirectly via streptavidin-biotin linkages; (iii) antibodies specific for a target molecule and conjugated to a bead, or microbead, or other solid phase support, which, in turn, is derivatized either directly or indirectly with moieties such as molecular tags or photosensitizers, or polymers containing the latter.

“Antigenic determinant,” or “pitope” means a site on the surface of a molecule, usually a protein, to which a single antibody molecule binds; generally a protein has several or many different antigenic determinants and reacts with antibodies of many different specificities. A preferred antigenic determinant is a phosphorylation site of a protein.

“Apoptosis,” or “programmed cell death,” means a process in which a cell destroys itself by a series of steps that includes the release of proteolytic enzymes, such as caspase enzymes. “Apoptotic pathway,” or equivalently “apoptotic signaling pathway,” refers to a particular series of steps leading to cell destruction wherein the steps include characteristic molecular events, such as phosphorylation or de-phosphorylation of particular proteins, the formation of complexes between particular proteins, the dissociation of complexes, and the like. “Apoptotic complex” refers to a protein-protein complex that forms as part of a particular apoptotic pathway. “Apoptotic status” is a term of relative degree that refers to a pattern or profile of relative amounts of one or more complexes in one or more apoptotic pathways that is correlated with cell death or with cell survival. For example, an amount of 14-3-3//BAD complex in excess of an amount of Bcl-2//BAD complex in the same sample suggests cell survival; thus, the value of apoptotic status is low. Particular levels or values, or measurement scales, for apoptotic status depend on several factors, including the cell types in a sample, whether a disease condition is present, the type of complexes being targeted, and the like. Levels or values of apoptotic status may be determined by reference to control samples that have known or normal levels of selected apoptotic complexes. As used herein, “status” in reference to a cancer patient means the apoptotic status of cells from a sample, specimen, or biopsy from a cancer patient. Such “status” may relate to disease determination or classification, prognosis, drug efficacy, patient responsiveness to therapy, whether adjuvant therapy is recommended, likelihood of recurrence of disease, or the like.

“BAD protein” means a human protein capable of forming a stable complex with a human 14-3-3 protein whenever phosphorylated at Ser-112 and/or Ser-155, and capable of forming a stable complex with a human Bcl-2 protein whenever there is no phosphate group attached to Ser-112 and Ser-155, such BAD protein having an amino acid sequence substantially identical to that described under NCBI accession number AAH01901 or in Strausberg et al, Proc. Natl. Acad. Sci., 99: 16899-16903 (2002). In one aspect, a BAD protein hereunder is at least eighty percent identical, and more preferably ninety percent identical, to the amino acid described under NCBI accession number AAH01901 or in Strausberg et al, Proc. Natl. Acad. Sci., 99: 16899-16903 (2002).

“Bcl-2 protein” means a human protein capable of forming a stable complex with a human BAD protein whenever the BAD protein has no phosphate group attached to Ser-112 or Ser-155, and having an amino acid sequence substantially identical to that described under NCBI accession number AAH17214 or in Strausberg et al, Proc. Natl. Acad. Sci., 99: 16899-16903 (2002). In one aspect, a Bcl-2 protein hereunder is at least eighty percent identical, and more preferably ninety percent identical, to the amino acid described under NCBI accession number AAH17214 or in Strausberg et al, Proc. Natl. Acad. Sci., 99: 16899-16903 (2002).

“Bcl-X_{L} protein” means a human protein capable of forming a stable complex with a human BAD protein whenever the BAD protein has no phosphate group attached to Ser-112 or Ser-155, and having an amino acid sequence substantially identical to that described under NCBI accession number NP_{—}620120 or in Oltvai et al, Cell, 74: 609-619 (1993). In one aspect, a Bcl-X_{L} protein hereunder is at least eighty percent identical, and more preferably ninety percent identical, to the amino acid described under NCBI accession number NP_{—}620120 or in Oltvai et al, Cell, 74: 609-619 (1993).

“BH3 only protein” means a human protein containing a BH3 domain, but not a BH1, BH2, or BH4 domains, and is capable of forming a stable complex with a Bcl-2 protein. Polypeptide domains, BH1, BH2, BH3, and BH4, are characteristic domains of the Bcl-2 family of proteins, which are described in the following references; Baell and Huang, Biochem. Pharmacology, 64: 851-863 (2002); Sattler et al, Science, 275: 983-985 (1997); Gross et al, Genes & Development, 13: 1899-1911 (1999); and Puthalakath et al, Cell Death and Differentiation, 9: 505-512 (2002); which are incorporated by reference. A BH3 domain is represented by the following consensus sequence of 12 amino acids: “-I-A-X₁-X₂-L-R-R-I-G-D-E-F-,” wherein X₁ is any amino acid and X₂ is a charged amino acid. Preferably, a BH3 domain comprises an amino acid sequence of the consensus sequence, or a sequence having from 1 to 4 conservative amino acid substitutions and/or deletions with respect to the consensus sequence, with the proviso that the leucine (L) in position five and the aspartic acid (D) in position 10 are not substituted or deleted. Exemplary BH3 only proteins include BID, BAD, BIK, BLK, HRK, BIM, NIP3, and NIX/BNIP3, descriptions of which are available from the National Center for Biotechnology Information (NCBI). A preferred BH3 only protein is BAD.

“Binding moiety” means any molecule to which molecular tags can be directly or indirectly attached that is capable of specifically binding to an analyte. Binding moieties include, but are not limited to, antibodies, antibody binding compositions, peptides, proteins, nucleic acids, and organic molecules having a molecular weight of up to 1000 daltons and consisting of atoms selected from the group consisting of hydrogen, carbon, oxygen, nitrogen, sulfur, and phosphorus. Preferably, binding moieties are antibodies or antibody binding compositions.

“Cancer” and “cancerous” refer to or describe the physiological condition in mammals that is typically characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, prostate cancer, vulval cancer, thyroid cancer, hepatic carcinoma and various types of head and neck cancer.

“Capillary-sized” in reference to a separation column means a capillary tube or channel in a plate or microfluidics device, where the diameter or largest dimension of the separation column is between about 25-500 microns, allowing efficient heat dissipation throughout the separation medium, with consequently low thermal convection within the medium.

“Chromatography” or “chromatographic separation” as used herein means or refers to a method of analysis in which the flow of a mobile phase, usually a liquid, containing a mixture of compounds, e.g. molecular tags, promotes the separation of such compounds based on one or more physical or chemical properties by a differential distribution between the mobile phase and a stationary phase, usually a solid. The one or more physical characteristics that form the basis for chromatographic separation of analytes, such as molecular tags, include but are not limited to molecular weight, shape, solubility, pKa, hydrophobicity, charge, polarity, and the like. In one aspect, as used herein, “high pressure (or performance) liquid chromatography” (“HPLC”) refers to a liquid phase chromatographic separation that (i) employs a rigid cylindrical separation column having a length of up to 300 mm and an inside diameter of up to 5 mm, (ii) has a solid phase comprising rigid spherical particles (e.g. silica, alumina, or the like) having the same diameter of up to 5 μm packed into the separation column, (iii) takes place at a temperature in the range of from 35° C. to 80° C. and at column pressure up to 150 bars, and (iv) employs a flow rate in the range of from 1 μL/min to 4 mL/min. Preferably, solid phase particles for use in HPLC are further characterized in (i) having a narrow size distribution about the mean particle diameter, with substantially all particle diameters being within 10% of the mean, (ii) having the same pore size in the range of from 70 to 300 angstroms, (iii) having a surface area in the range of from 50 to 250 m²/g, and (iv) having a bonding phase density (i.e. the number of retention ligands per unit area) in the range of from 1 to 5 per nm². Exemplary reversed phase chromatography media for separating molecular tags include particles, e.g. silica or alumina, having bonded to their surfaces retention ligands, such as phenyl groups, cyano groups, or aliphatic groups selected from the group including C₈ through C₁₈. Chromatography in reference to the invention includes “capillary electrochromatography” (“CEC”), and related techniques. CEC is a liquid phase chromatographic technique in which fluid is driven by electroosmotic flow through a capillary-sized column, e.g. with inside diameters in the range of from 30 to 100 μm. CEC is disclosed in Svec, Adv. Biochem. Eng. Biotechnol. 76: 1-47 (2002); Vanhoenacker et al, Electrophoresis, 22: 4064-4103 (2001); and like references. CEC column may use the same solid phase materials as used in conventional reverse phase HPLC and additionally may use so-called “monolithic” non-particular packings. In some forms of CEC, pressure as well as electroosmosis drives an analyte-containing solvent through a column.

“Complex” as used herein means an assemblage or aggregate of molecules in direct or indirect contact with one another. As used herein, “contact,” or more particularly, “direct contact” in reference to a complex of molecules, or in reference to specificity or specific binding, means two molecules are close enough that weak noncovalent chemical interactions, such as Van der Waal forces, hydrogen bonding, ionic and hydrophobic interactions, and the like, dominate the interaction of the molecules. Generally, a complex of molecules is stable in that under assay conditions the complex is thermodynamically more favorable than a non-aggregated state of its component molecules. As used herein, “complex” usually refers to a stable aggregate of two or more proteins, and is equivalently referred to as a “protein-protein complex.” As used herein, an “intracellular complex” or “intracellular protein-protein complex,” refers to a complex of proteins normally found in the cytoplasm or nucleus of a biological cell, and may include complexes of one or more intracellular proteins and a surface membrane receptor. Exemplary intracellular proteins that may be part of such complexes include, but are not limit to, PI3K proteins, Grb2 proteins, Grb7 proteins, Shc proteins, and Sos proteins, Src proteins, Cbl proteins, PLCγ proteins, Shp2 proteins, GAP proteins, Nck proteins, Vav proteins, and Crk proteins. In one aspect, such complexes include PI3K or Shc proteins. In another aspect, a complex is a stable aggregate comprising two proteins, or from 2 to 4 proteins, or from 2 to 6 proteins. As used herein, a “signaling complex” is an intracellular protein-protein complex that is a component of a signaling pathway. Exemplary signaling complexes are listed in Tables IIIA-B. In one aspect, the term “complex” includes complexes of nuclear steroid or fatty acid receptors and their co-factors, e.g. peroxisome proliferator-activated receptors.

“Dimer” means a complex of two or more proteins that may be the same or different, including membrane-bound receptors. Dimers of identical proteins are referred to as “homodimers” and dimers of different proteins are referred to as “heterodimers.” Dimers usually consist of two proteins in contact with one another. Dimers may be created in a cell surface membrane or in the interior of a cell by passive processes, such as Van der Waal interactions, and the like, as described above in the definition of “complex,” or dimers may be created by active processes, such as by ligand-induced dimerization, phosphorylations, covalent linkages, interaction with intracellular components, or the like, e.g. Schlessinger, Cell, 103: 211-225 (2000).

“Disease status” inlcudes, but is not limited to, the following features: likelihood of contracting a disease, presence or absence of a disease, prognosis of disease severity, and likelihood that a patient will respond to treatment by a particular therapeutic agent that acts through an intracellular complex. In regard to cancer, “disease status,” or “cancer status,” further includes detection of precancerous or cancerous cells or tissues, the selection of patients that are likely to respond to treatment by a therapeutic agent that acts through one or more intracellular complexes, such as one or more protein-protein dimers, and the ameliorative effects of treatment with such therapeutic agents.

“ErbB receptor” or “Her receptor” is a receptor protein tyrosine kinase which belongs to the ErbB receptor family and includes EGFR (“Her1”), ErbB2 (“Her2”), ErbB3 (“Her3”) and ErbB4 (“Her4”) receptors. The ErbB receptor generally comprises an extracellular domain, which may bind an ErbB ligand; a lipophilic transmembrane domain; a conserved intracellular tyrosine kinase domain; and a carboxyl-terminal signaling domain harboring several tyrosine. residues which can be phosphorylated. The ErbB receptor may be a native sequence ErbB receptor or an amino acid sequence variant thereof. Preferably the ErbB receptor is native sequence human ErbB receptor. In one aspect, ErbB receptor includes truncated versions of Her receptors, including but not limited to, EGFRvIII and p95Her2, disclosed in Chu et al, Biochem. J., 324: 855-861 (1997); Xia et al, Oncogene, 23: 646-653 (2004); and the like.

The terms “ErbB 1”, “epidermal growth factor receptor” and “EGFR” and “Her1” are used interchangeably herein and refer to native sequence EGFR as disclosed, for example, in Carpenter et al. Ann. Rev. Biochem. 56:881-914 (1987), including variants thereof (e.g. a deletion mutant EGFR as in Humphrey et al. PNAS (USA) 87:4207-4211 (1990)). erbB1 refers to the gene encoding the EGFR protein product. Examples of antibodies which bind to EGFR include MAb 579 (ATCC CRL RB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225) and reshaped human 225 (H225) (see, WO 96/40210, Imclone Systems Inc.).

“Her2”, “ErbB2” “c-Erb-B2” are used interchangeably. Unless indicated otherwise, the terms “ErbB2” “c-Erb-B2” and “Her2” when used herein refer to the human protein. The human ErbB2 gene and ErbB2 protein are, for example, described in Semba et al., PNAS (USA) 82:6497-650 (1985)and Yamamoto et al. Nature 319:230-234 (1986) (Genebank accession number X03363). Examples of antibodies that specifically bind to Her2 are disclosed in U.S. Pat. Nos. 5,677,171; 5,772,997; Fendly et al, Cancer Res., 50: 1550-1558 (1990); and the like.

“ErbB3” and “Her3” refer to the receptor polypeptide as disclosed, for example, in U.S. Pat. Nos. 5,183,884 and 5,480,968 as well as Kraus et al. PNAS (USA) 86:9193-9197 (1989), including variants thereof. Examples of antibodies which bind Her3 are described in U.S. Pat. No. 5,968,511, e.g. the 8B8 antibody (ATCC HB 12070).

The terms “ErbB4” and “Her4” herein refer to the receptor polypeptide as disclosed, for example, in EP Pat Appln No 599,274; Plowman et al., Proc. Natl. Acad. Sci. USA, 90:1746-1750 (1993); and Plowman et al., Nature, 366:473-475 (1993), including variants thereof such as the Her4 isoforms disclosed in WO 99/19488.

“Insulin-like growth factor-1 receptor” or “IGF-IR” means a human receptor tyrosine kinase substantially identical to those disclosed in Ullrich et al, EMBO J., 5: 2503-2512 (1986) or Steele-Perkins et al, J. Biol. Chem., 263: 11486-11492 (1988).

“I-kB protein,” or “inhibitor of NF-kB protein,” means a human protein capable of forming a stable complex with a human NF-KB protein whenever the I-kB protein is in a partially phosphorylated state. In one aspect, an I-kB protein has an amino acid sequence substantially identical to that described under NCBI accession number O00221 or in Baeuerle and Baltimore, Science, 242: 540-546 (1988).

“Isolated” in reference to a polypeptide or protein means substantially separated from the components of its natural environment. Preferably, an isolated polypeptide or protein is a composition that consists of at least eighty percent of the polypeptide or protein identified by sequence on a weight basis as compared to components of its natural environment; more preferably, such composition consists of at least ninety-five percent of the polypeptide or protein identified by sequence on a weight basis as compared to components of its natural environment; and still more preferably, such composition consists of at least ninety-nine percent of the polypeptide or protein identified by sequence on a weight basis as compared to components of its natural environment. Most preferably, an isolated polypeptide or protein is a homogeneous composition that can be resolved as a single spot after conventional separation by two-dimensional gel electrophoresis based on molecular weight and isoelectric point. Protocols for such analysis by conventional two-dimensional gel electrophoresis are well known to one of ordinary skill in the art, e.g. Hames and Rickwood, Editors, Gel Electrophoresis of Proteins: A Practical Approach (IRL Press, Oxford, 1981); Scopes, Protein Purification (Springer-Verlag, New York, 1982); Rabilloud, Editor, Proteome Research: Two-Dimensional Gel Electrophoresis and Identification Methods (Springer-Verlag, Berlin, 2000).

“Kit” refers to any delivery system for delivering materials. In the context of reaction assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second container contains probes.

“NF-kB protein,” or “nuclear factor kappa B protein,” means a human protein that is a member of a group of related transcription factors consisting of Rel (also known as c-Rel), RelA (also known as p65 and NF-kB3), RelB, NF-KB1 (also known as p50), and NF-kB2 (also known as p52). In one aspect, an NF-kB protein is a p50-RelA dimer and is capable of forming a stable complex with a human I-kB protein whenever the I-kB protein is in a partially phosphorylated state. NF-kB proteins and their relationship to I-kB proteins are described in the following references: Karin et al, Nature Reviews Drug Discovery, 3: 17-26 (2004); Karin et al, Nature Reviews Cancer, 2: 301-310 (2002); Li et al, Nature Reviews Immunology, 2: 725-734 (2002); and Baldwin, Annu. Rev. Immunol., 14: 649-681 (1996).

“Percent identical,” or like term, used in respect of the comparison of a reference sequence and another sequence (i.e. a “candidate” sequence) means that in an optimal alignment between the two sequences, the candidate sequence is identical to the reference sequence in a number of subunit positions equivalent to the indicated percentage, the subunits being nucleotides for polynucleotide comparisons or amino acids for polypeptide comparisons. As used herein, an “optimal alignment” of sequences being compared is one that maximizes matches between subunits and minimizes the number of gaps employed in constructing an alignment. Percent identities may be determined with commercially available implementations of algorithms described by Needleman and Wunsch, J. Mol. Biol., 48: 443-453 (1970)(“GAP” program of Wisconsin Sequence Analysis Package, Genetics Computer Group, Madison, Wis.). Other software packages in the art for constructing alignments and calculating percentage identity or other measures of similarity include the “BestFit” program, based on the algorithm of Smith and Waterman, Advances in Applied Mathematics, 2: 482-489 (1981) (Wisconsin Sequence Analysis Package, Genetics Computer Group, Madison, Wis.). In other words, for example, to obtain a polypeptide having an amino acid sequence at least 95 percent identical to a reference amino acid sequence, up to five percent of the amino acid residues in the reference sequence many be deleted or substituted with another amino acid, or a number of amino acids up to five percent of the total amino acid residues in the reference sequence may be inserted into the reference sequence. These alterations of the reference sequence many occur at the amino or carboxy terminal positions of the reference amino acid sequence or anywhere between those terminal positions, interspersed either individually among residues in the reference sequence of in one or more contiguous groups with in the references sequence. It is understood that in making comparisons with reference sequences of the invention that candidate sequence may be a component or segment of a larger polypeptide or polynucleotide and that such comparisons for the purpose computing percentage identity is to be carried out with respect to the relevant component or segment.

“Phosphatidylinositol 3 kinase protein,” or equivalently a “PI3K protein,” means a human intracellular protein of the set of human proteins describe under NCBI accession numbers NP_{—}852664, NP_{—}852556, and NP_{—}852665, and proteins having amino acid sequences substantially identical thereto.

“Platelet-derived growth factor receptor” or “PDGFR” means a human receptor tyrosine kinase protein that is substantially identical to PDGFRα: or PDGFRβ or variants thereof, described in Heldin et al, Physiological Reviews, 79: 1283-1316 (1999). In one aspect, the invention includes determining the status of cancers, pre-cancerous conditions, fibrotic or sclerotic conditions by measuring one or more dimers of the following group: PDGFRα homodimers, PDGFRβ homodimers, and PDGFRα-PDGFRβ heterodimers. In particular, fibrotic conditions include lung or kidney fibrosis, and sclerotic conditions include atherosclerosis. Cancers include, but are not limited to, breast cancer, colorectal carcinoma, glioblastoma, and ovarian carcinoma. Reference to “PDGFR” alone is understood to mean “PDGFRα” or “PDGFRβ.”

“Polypeptide” refers to a class of compounds composed of amino acid residues chemically bonded together by amide linkages with elimination of water between the carboxy group of one amino acid and the amino group of another amino acid. A polypeptide is a polymer of amino acid residues, which may contain a large number of such residues. Peptides are similar to polypeptides, except that, generally, they are comprised of a lesser number of amino acids. Peptides are sometimes referred to as oligopeptides. There is no clear-cut distinction between polypeptides and peptides. For convenience, in this disclosure and claims, the term “polypeptide” will be used to refer generally to peptides and polypeptides. The amino acid residues may be natural or synthetic.

“Protein” refers to a polypeptide, usually synthesized by a biological cell, folded into a defined three-dimensional structure. Proteins are generally from about 5,000 to about 5,000,000 or more in molecular weight, more usually from about 5,000 to about 1,000,000 molecular weight, and may include posttranslational modifications, such acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, farnesylation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, phosphorylation, prenylation, racemization, selenoylation, sulfation, and ubiquitination, e.g. Wold, F., Post-translational Protein Modifications: Perspectives and Prospects, pgs. 1-12 in Post-translational Covalent Modification of Proteins, B. C. Johnson, Ed., Academic Press, New York, 1983. Proteins include, by way of illustration and not limitation, cytokines or interleukins, enzymes such as, e.g., kinases, proteases, galactosidases and so forth, protamines, histones, albumins, immunoglobulins, scleroproteins, phosphoproteins, mucoproteins, chromoproteins, lipoproteins, nucleoproteins, glycoproteins, T-cell receptors, proteoglycans, and the like.

“Receptor tyrosine kinase activation status,” “receptor tyrosine kinase pathway activation status,” or “RTK pathway activation status,” or like terms, means a pattern or profile of relative amounts of one or more signaling complexes in one or more pathways that are associated with an RTK. For example, an amount of PI3K//RTK complex in excess of an amount of Shc//Grb2 complex in the same sample suggests a predominant activation of the PI3K-Akt pathway over the Ras-MAPK pathway. Particular levels or values, or measurement scales, for RTK pathway activation status depend on several factors, including the cell types in a sample, whether a disease condition is present, the type of complexes being targeted within each pathway, and the like. Levels or values of RTK pathway activation status may be determined by reference to control or reference samples that have known or normal levels of selected complexes under standard or predetermined conditions. As used herein, “status” in reference to a cancer patient means the RTK pathway activation status of cells from a sample, specimen, or biopsy from a patient, particularly a cancer patient. Such “status” may relate to disease determination or classification, prognosis, drug efficacy, patient responsiveness to therapy, whether adjuvant therapy is recommended, likelihood of recurrence of disease, or the like.

“Receptor tyrosine kinase,” or “RTK,” means a human receptor protein having intracellular kinase activity and being selected from the RTK family of proteins described in Schlessinger, Cell, 103: 211-225 (2000); and Blume-Jensen and Hunter (cited above). “Receptor tyrosine kinase dimer” means a complex in a cell surface membrane comprising two receptor tyrosine kinase proteins. In some aspects, a receptor tyrosine kinase dimer may comprise two covalently linked receptor tyrosine kinase proteins. Exemplary RTK dimers are listed in Table I. RTK dimers of particular interest are Her receptor dimers and VEGFR dimers.

“Reference sample” means one or more cell, xenograft, or tissue samples that are representative of a normal or non-diseased state to which measurements on patient samples are compared to determine whether a receptor complex is present in excess or is present in reduced amount in the patient sample. The nature of the reference sample is a matter of design choice for a particular assay and may be derived or determined from normal tissue of the patient him- or herself, or from tissues from a population of healthy individuals. Preferably, values relating to amounts of receptor complexes in reference samples are obtained under essentially identical experimental conditions as corresponding values for patient samples being tested. Reference samples may be from the same kind of tissue as that the patient sample, or it may be from different tissue types, and the population from which reference samples are obtained may be selected for characteristics that match those of the patient, such as age, sex, race, and the like. Typically, in assays of the invention, amounts of receptor complexes on patient samples are compared to corresponding values of reference samples that have been previously tabulated and are provided as average ranges, average values with standard deviations, or like representations.

“Sample” or “tissue sample” or “patient sample” or “patient cell or tissue sample” or “specimen” each means a collection of similar cells obtained from a tissue of a subject or patient. The source of the tissue sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; or cells from any time in gestation or development of the subject. The tissue sample may contain compounds which are not naturally intermixed with the tissue in nature such as preservatives, anticoagulants, buffers, fixatives, nutrients, antibiotics, or the like. In one aspect of the invention, tissue samples or patient samples are fixed, particularly conventional formalin-fixed paraffin-embedded samples. Such samples are typically used in an assay for receptor complexes in the form of thin sections, e.g. 3-10 μm thick, of fixed tissue mounted on a microscope slide, or equivalent surface. Such samples also typically undergo a conventional re-hydration procedure, and optionally, an antigen retrieval procedure as a part of, or preliminary to, assay measurements.

A “separation profile” in reference to the separation of molecular tags means a chart, graph, curve, bar graph, or other representation of signal intensity data versus a parameter related to the molecular tags, such as retention time, mass, or the like, that provides a readout, or measure, of the number of molecular tags of each type produced in an assay. A separation profile may be an electropherogram, a chromatogram, an electrochromatogram, a mass spectrogram, or like graphical representation of data depending on the separation technique employed. A “peak” or a “band” or a “zone” in reference to a separation profile means a region where a separated compound is concentrated. There may be multiple separation profiles for a single assay if, for example, different molecular tags have different fluorescent labels having distinct emission spectra and data is collected and recorded at multiple wavelengths. In one aspect, released molecular tags are separated by differences in electrophoretic mobility to form an electropherogram wherein different molecular tags correspond to distinct peaks on the electropherogram. A measure of the distinctness, or lack of overlap, of adjacent peaks in an electropherogram is “electrophoretic resolution,” which may be taken as the distance between adjacent peak maximums divided by four times the larger of the two standard deviations of the peaks. Preferably, adjacent peaks have a resolution of at least 1.0, and more preferably, at least 1.5, and most preferably, at least 2.0. In a given separation and detection system, the desired resolution may be obtained by selecting a plurality of molecular tags whose members have electrophoretic mobilities that differ by at least a peak-resolving amount, such quantity depending on several factors well known to those of ordinary skill, including signal detection system, nature of the fluorescent moieties, the diffusion coefficients of the tags, the presence or absence of sieving matrices, nature of the electrophoretic apparatus, e.g. presence or absence of channels, length of separation channels, and the like. Electropherograms may be analyzed to associate features in the data with the presence, absence, or quantities of molecular tags using analysis programs, such as disclosed in Williams et al, U.S. patent publication 2003/0170734 A1.

“SHC” (standing for “Src homology 2/α-collagen-related”) means any one of a family of adaptor proteins (66, 52, and 46 kDalton) in RTK signaling pathways substantially identical to those described in Pelicci et al, Cell, 70: 93-104 (1992). In one aspect, SHC means the human versions of such adaptor proteins.

“Signaling pathway” or “signal transduction pathway” means a series of molecular events usually beginning with the interaction of cell surface receptor with an extracellular ligand or with the binding of an intracellular molecule to a phosphorylated site of a cell surface receptor that results in a regulation of gene expression in the nucleus of a cell. “Ras-MAPK pathway” means a signaling pathway that includes the phosphorylation of a MAPK protein subsequent to the formation of a Ras-GTP complex. “PI3K-Akt pathway” means a signaling pathway that includes the phosphorylation of an Akt protein by a PI3K protein.

“Specific” or “specificity” in reference to the binding of one molecule to another molecule, such as a binding compound, or probe, for a target analyte or complex, means the recognition, contact, and formation of a stable complex between the probe and target, together with substantially less recognition, contact, or complex formation of the probe with other molecules. In one aspect, “specific” in reference to the binding of a first molecule to a second molecule means that to the extent the first molecule recognizes and forms a complex with another molecules in a reaction or sample, it forms the largest number of the complexes with the second molecule. In one aspect, this largest number is at least fifty percent of all such complexes form by the first molecule. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like.

As used herein, the term “spectrally resolvable” in reference to a plurality of fluorescent labels means that the fluorescent emission bands of the labels are sufficiently distinct, i.e. sufficiently non-overlapping, that molecular tags to which the respective labels are attached can be distinguished on the basis of the fluorescent signal generated by the respective labels by standard photodetection systems, e.g. employing a system of band pass filters and photomultiplier tubes, or the like, as exemplified by the systems described in U.S. Pat. Nos. 4,230,558; 4,811,218, or the like, or in Wheeless et al, pgs. 21-76, in Flow Cytometry: Instrumentation and Data Analysis (Academic Press, New York, 1985).

“Substantially identical” in reference to proteins or amino acid sequences of proteins in a family of related proteins that are being compared means either that one protein has an amino acid sequence that is at least fifty percent identical to the other protein or that one protein is an isoform or splice variant of the same gene as the other protein. In one aspect, substantially identical means one protein, or amino acid sequence thereof, is at least eighty percent identical to the other protein, or amino acid sequence thereof.

“VEGF receptor” or “VEGFR” as used herein refers to a cellular receptor for vascular endothelial growth factor (VEGF), ordinarily a cell-surface receptor found on vascular endothelial cells, as well as variants thereof which retain the ability to bind human VEGF. VEGF receptors include VEGFR1 (also known as Flt1), VEGFR2 (also know as Flk1 or KDR), and VEGFR3 (also known as Flt4). These receptors are described in DeVries et al., Science 255:989 (1992); Shibuya et al., Oncogene 5:519 (1990); Matthews et al., Proc. Nat. Acad. Sci. 88:9026 (1991); Terman et al., Oncogene 6:1677 (1991); Terman et al., Biochem. Biophys. Res. Commun. 187:1579 (1992). Dimers of VEGF receptors are described in Shibuya, Cell Structure and Function, 26: 25-35 (2001); and Ferrara et al, Nature Medicine, 9: 669-676 (2003).

### DETAILED DESCRIPTION OF THE INVENTION

The invention provides a method of using intracellular complexes as biomarkers for the status of a disease or other physiological conditions in a biological organism, particularly a cancer status in a human. In one aspect, intracellular complexes are measured directly from patient samples; that is, measurements are made without culturing, formation of xenografts, or the use of like techniques, that require extra labor and expense and that may introduce artifacts and/or noise into the measurement process. In a particular aspect of the invention, measurements of one or more intracellular complexes are made directly on tissue lysates of frozen patient samples or on sections of fixed patient samples. In a preferred embodiment, one or more intracellular complexes are measured in sections of formalin-fixed paraffin-embedded (FFPE) samples.

In another aspect, the invention provides an indirect measurement of the phosphorylation of intracellular proteins through the measurement of complexes that depend on such posttranslational modifications for their formation.

In another aspect, the invention provides a method for measuring a plurality of intracellular complexes simultaneously in the same assay reaction mixture. Preferably, such complexes are measured using binding compounds having one or more molecular tags releasably attached, such that after binding to a protein in a complex, the molecular tags may be released and separated from the reaction, or assay, mixture for detection and/or quantification.

In another aspect, the invention provides a method for determining a disease status of a patient comprising the following steps: measuring an amount of each of one or more intracellular protein-protein complexes in a patient sample; comparing each such amount to its corresponding amount from a reference sample; and correlating differences in the amounts from the patient sample and the respective corresponding amounts from the reference sample to the presence or severity of a disease condition in the patient. In a preferred embodiment, the step of measuring comprising the steps of: (i) providing one or more binding compounds specific for a protein of each of the one or more complexes, such that each binding compound has one or more molecular tags each attached thereto by a cleavable linkage, and such that the one or more molecular tags attached to different binding compounds have different separation characteristics so that upon separation molecular tags from different binding compounds form distinct peaks in a separation profile; (ii) mixing the binding compounds and the one or more complexes such that binding compounds specifically bind to their respective proteins of the complexes to form detectable complexes; (iii) cleaving the cleavable linkage of each binding compound forming detectable complexes, and (iv) separating and identifying the released molecular tags to determine the presence or absence or the amount of the one or more complexes of proteins.

In another aspect, the step of measuring the amounts of one or more complexes comprising the following steps: (i) providing for each of the one or more complexes a cleaving probe specific for a first protein in each of the one or more complexes, each cleaving probe having a cleavage-inducing moiety with an effective proximity; (ii) providing one or more binding compounds specific for a second protein of each of the one or more complexes, such that each binding compound has one or more molecular tags each attached thereto by a cleavable linkage, and such that the one or more molecular tags attached to different binding compounds have different separation characteristics so that upon separation molecular tags from different binding compounds form distinct peaks in a separation profile; (iii) mixing the cleaving probes, the binding compounds, and the one or more complexes such that cleaving probes specifically bind to first proteins of the complexes and binding compounds specifically bind to the second proteins of the complexes and such that cleavable linkages of the binding compounds are within the effective proximity of cleavage-inducing moieties of the cleaving probes so that molecular tags are released; and (iv) separating and identifying the released molecular tags to determine the presence or absence or the amount of the one or more complexes of proteins. Preferably, complexes and first and second proteins are selected from the complexes listed in Tables IIA and IIB.

In another aspect, the invention is implemented by methods employing cleaving probes that generate a locally acting cleaving agent and binding compounds labeled with releasable molecular tags that are released by the cleaving agent. Complex formation is detected by designing cleaving probes and binding compounds such that at least one cleaving probe specifically binds to a different component of a complex than at least one of the binding compounds. In this manner, molecular tags of a predetermined type are released only when a complex is formed.

In another aspect of the invention, a biological specimen, which comprises a mixed cell population suspected of containing the rare cell of interest is obtained from a patient. A sample is then prepared by mixing the biological specimen with magnetic particles which are coupled to a biospecific ligand specifically reactive with an antigen on the rare cell that is different from or not found on blood cells (referred to herein as a “capture antigen”), so that other sample components may be substantially removed. The sample is subjected to a magnetic field which is effective to separate cells labeled with the magnetic particles, including the rare cells of interest, if any are present in the specimen. The cell population so isolated is then analyzed using molecular tags conjugated to binding moieties specific for intracellular complexes to determine the presence and/or number of rare cells. In a preferred embodiment the magnetic particles used in this method are colloidal magnetic nanoparticles. Preferably, such rare cell populations are circulating epithelial cells, which may be isolated from patient's blood using epithelial-specific capture antigens, e.g. as disclosed in Hayes et al, International J. of Oncology, 21: 1111-1117 (2002); Soria et al, Clinical Cancer Research, 5: 971-975 (1999); Ady et al, British J. Cancer, 90: 443-448 (2004); which are incorporated by reference. In particular, monoclonal antibody BerEP4 (Dynal A.S., Oslo, Norway) may be used to capture human epithelial cells with magnetic particles.

In another aspect, the invention provides a method for determining a cancer status of a patient comprising the following steps: (i) immunomagnetically isolating a patient sample comprising circulating epithelial cells by contacting a sample of patient blood with one or more antibody compositions, each antibody composition being specific for a capture antigen and being attached to a magnetic particle; (ii) measuring an amount of each of one or more intracellular complexes in the patient sample; comparing each such amount to its corresponding amount from a reference sample; and correlating differences in the amounts from the patient sample and the respective corresponding amounts from the reference sample to the presence or severity of a cancer condition in the patient. In a preferred embodiment, the step of measuring comprises the steps of: (i) providing one or more binding compounds specific for a protein of each of the one or more intracellular complexes, such that each binding compound has one or more molecular tags each attached thereto by a cleavable linkage, and such that the one or more molecular tags attached to different binding compounds have different separation characteristics so that upon separation molecular tags from different binding compounds form distinct peaks in a separation profile; (ii) mixing the binding compounds and the one or more intracellular complexes such that binding compounds specifically bind to their respective proteins of the one or more intracellular complexes to form detectable complexes; (iii) cleaving the cleavable linkage of each binding compound forming detectable complexes, and (iv) separating and identifying the released molecular tags to determine the presence or absence or the amount of the one or more intracellular complexes.

### Exemplary Proteins Forming Detectable Complexes

In one aspect, assays of the invention may be used to determine whether one or more apoptotic pathways are activated by simultaneously measuring protein-protein complexes. Of particular interest are protein-protein complexes that include, but are not limited to, the proteins of the following Tables. Preferably, the human forms of the following proteins and protein families are intended.

TABLE I
Exemplary Proteins Forming Intracellular Complexes
in Apoptotic Pathways

| | Comments/NCBI | |
|---|---|---|
| Abbreviation | references | Reference |
| 14-3-3 protein | BC003047 | Aitken et al, Biochem. Soc. |
| | | Trans., 30: 351-360 (2002); |
| | | Subramanian et al, Exp. Cell |
| | | Res., 271: 142-151 (2001) |
| BAD | BC001901 | Ottilie et al, J. Biol. Chem., 272: |
| | AF031523 | 30866-30872 (1997); Schurmann |
| | BT006678 | et al, Mol. Cell. Biol. 20: 453-461 |
| | | (2000); Baell et al, Biochem. |
| | | Pharmacology, 64: 851-863 |
| | | (2002) |
| BID | | Gross et al, Genes & |
| | | Development, 13: 1899-1911 |
| | | (1999); Baell et al (cited above) |
| BIK/NBK | | Gross et al (1999, cited above); |
| | | Baell et al (cited above) |
| Blk | | Gross et al (1999, cited above); |
| | | Baell et al (cited above) |
| Hrk | | Gross et al (1999, cited above) |
| BIM/BOD | | Gross et al (1999, cited above); |
| | | Baell et al (cited above) |
| NIP3 | | Gross et al (1999, cited above); |
| | | Baell et al (cited above) |
| NIX/BNIP3 | | Gross et al (1999, cited above); |
| | | Baell et al (cited above) |
| Noxa | | Antonsson, Cell Tissue Res, 306: |
| | | 347-361 (2001) |
| PUMA | | Antonsson (cited above) |
| BAX | | Antonsson (cited above); Gross et |
| | | al, Mol. Cell. Biol. 20: 3125-3136 |
| | | (2000); Baell et al (cited |
| | | above) |
| BAK | | Gross et al (1999, cited above) |
| Bcl-X_{S} | | Gross et al (1999, cited above) |
| BOK/MTD | | Gross et al (1999, cited above) |
| Bcl-G | | Puthalakath et al, Cell Death and |
| | | Differentiation, 9: 505-512 |
| | | (2002) |
| Bcl-2 | M14745 | Srivastava et al, Proc. Natl. Acad. |
| | | Sci., 96: 3775-3780 (1999); Baell |
| | | et al (cited above) |
| Bcl-X_{L} | | Gross et al (1999, cited above) |
| Bcl-W | | Gross et al (1999, cited above) |
| MCL-1 | | Gross et al (1999, cited above) |
| A1/Bfl-1 | | Gross et al (1999, cited above) |
| BOO/DIVA | | Gross et al (1999, cited above) |
| NR-13 | | Gross et al (1999, cited above) |
| NF-kB | | |
| I-kB | | |
| caspase-9 | | Creagh et al, Biochemical |
| | | Society Transactions, 29: 696-702 |
| | | (2001); |
| FADD | | Creagh et al (cited above) |
| TRADD | | Creagh et al (cited above) |
| Apaf-1 | | Creagh et al (cited above) |

TABLE IIA
Exemplary Protein-Protein Complexes in Apoptotic Pathways
(where “protein 1//protein 2” indicates a complex comprising protein 1 and protein 2)

| | | | | |
|---|---|---|---|---|
| 14-3-3//BAD | BID//BAX | BAX//BAX | Bcl-X_{L}//BAD | Bcl-2//BAD |
| 14-3-3//BID | BID//BAK | BAX//Bcl-2 | Bcl-X_{L}//BIK | Bcl-2//BIK |
| NF-kB//I-kB | BID//Bcl-2 | | Bcl-X_{L}//BID | Bcl-2//BID |
| FADD//caspase-9 | BID//Bcl-X_{L} | | Bcl-X_{L}//Hrk | Bcl-2//Hrk |
| TRADD//caspase-9 | BID//A1/Bfl-1 | | Bcl-X_{L}//BIM | Bcl-2//BIM |
| Apaf-1//caspase-9 | | | Bcl-X_{L}//Noxa | Bcl-2//Noxa |
| | | | Bcl-X_{L}//Bmf | Bcl-2//Bmf |
| | | | Bcl-X_{L}//Puma | Bcl-2//Puma |
| | | | Bcl-X_{L}//Bcl-G | Bcl-2//Bcl-G |
| | | | Bcl-X_{L}//NIP3 | Bcl-2//NIP3 |
| | | | Bcl-X_{L}//Nix | Bcl-2//Nix |

TABLE IIB
Exemplary RTK Dimers and Intracellular Complexes
(here “protein 1//protein 2” indicates a complex comprising protein 1 and protein 2)

| RTK Dimer | Downstream Complexes |
|---|---|
| Her1-Her1 | Her1//Shc, Grb2//Sos, Her1//Grb7, Her1//RasGAP |
| Her1-Her2 | Her1//Shc, Grb2//Shc, Her2//Shc, Grb2//Sos, 14-3-3//Bad, |
| | Her1//RasGAP |
| Her1-Her3 | Her3//PI3K, Her3//Shc, Her3//Grb7, Her1//Shc, Grb2//Sos, 14-3-3//Bad, |
| | Her1//RasGAP |
| Her1-Her4 | Her3//PI3K, Her1//Shc, Grb2//Sos, Her1//RasGAP |
| Her2-Her2 | Her2//Shc, Grb2//Sos, 14-3-3//Bad, Her1//RasGAP |
| Her2-Her3 | Her3//PI3K, Her3//Shc, Her3//Grb7, Grb2//Shc, Her2//Shc, Grb2//Sos, |
| | 14-3-3//Bad, Her1//RasGAP |
| Her2-Her4 | Her3//PI3K, Grb2//Shc, Her2//Shc, Grb2//Sos, 14-3-3//Bad; YAP//Her4, |
| | Her1//RasGAP |
| Her3-Her4 | Her3//PI3K, Her3//Shc, Her3//Grb7, YAP//Her4, Her1//RasGAP |
| Her4-Her4 | Her3//PI3K, YAP//Her4, Her1//RasGAP |
| IGF-1R (covalent | IGF-1R//PI3K, IGF-1R//Shc; IGFR//IRS1 |
| homodimers) | |
| VEGFR1 (Flt1)- | VEGFR//Shc; VEGFR//PI(3)K; VEGFR//Src; VEGFR//FRS2 |
| VEGFR2(KDR) | |
| VEGFR2(KDR)- | VEGFR//Shc; VEGFR//PI(3)K; VEGFR//Src; VEGFR//FRS2 |
| VEGFR2(KDR) | |
| PDGFRa-PDGFRa | PDGFRa//Crk, PDGFR//Grb2; PDGFR//Grb7; PDGFR//Nck; |
| | PDGFR//Shc;, PDGFR//STAT5 |
| PDGFRa-PDGFRb | PDGFRa//Crk, PDGFRb//GAP, PDGFR//Grb2; PDGFR//Grb7; |
| | PDGFR//Nck; PDGFR//Shc, PDGFR//Shp2; PDGFR//RasGAP, |
| | PDGFR//STAT5 |
| PDGFRb-PDGFRb | PDGFRb//GAP, PDGFR//Grb2; PDGFR//Grb7; PDGFR//Nck; |
| | PDGFR//Shc, PDGFR//Shp2, PDGFR//RasGAP;, PDGFR//STAT5 |
| Kit/SCFR(homodimers) | Kit//Shp-1; Kit//p85PI(3)K; Kit//Grb2; Kit//CRKL |
| FGFR (particularly FGFR1 | FGFR//PLCg1; FGFR//Crk; FGFR//FRS2; FGFR//Shp2; FGFR//Shb |
| homodimers) | |
| NGFR(TrkA)-NGFR(TrkA) | Trk//p75NTR; Trk//PI(3)K |
| | Shc//Grb2; Grb2//SOS Shc//Her1; Shc//Her2; Shc//Her3; PI3K//Her1; |
| | IGF-1R//PI3K; IGF-1R//Shc; Erk//Rsk; 14-3-3//FKHRL1; Cyclin |
| | D1//Cdk4; 14-3-3//tuberin; 14-3-3//Cdc25C; 14-3-3σ//Cdc2; |
| | RXRα//CAR; RXRα//PPARα; RXRα//PXR; Hsp90//Akt1 |

### Preparation of Samples

Samples containing molecular complexes may come from a wide variety of sources for use with the present invention to relate receptor complexes populations to disease status or health status, including cell cultures, animal or plant tissues, patient biopsies, or the like. Preferably, samples are human patient samples. Samples are prepared for assays of the invention using conventional techniques, which may depend on the source from which a sample is taken.

A. Solid Tissue Samples.

For biopsies and medical specimens, guidance is provided in the following references: Bancroft J D & Stevens A, eds. Theory and Practice of Histological Techniques (Churchill Livingstone, Edinburgh, 1977); Pearse, Histochemistry. Theory and applied. 4^{th} ed. (Churchill Livingstone, Edinburgh, 1980).

In the area of cancerous disease status, examples of patient tissue samples that may be used include, but are not limited to, breast, prostate, ovary, colon, lung, endometrium, stomach, salivary gland or pancreas. The tissue sample can be obtained by a variety of procedures including, but not limited to surgical excision, aspiration or biopsy. The tissue may be fresh or frozen. In one embodiment, assays of the invention are carried out on tissue samples that have been fixed and embedded in paraffin or the like; therefore, in such embodiments a step of deparaffination is carried out. A tissue sample may be fixed (i.e. preserved) by conventional methodology [See e.g., “Manual of Histological Staining Method of the Armed Forces Institute of Pathology,” 3^{rd} edition (1960) Lee G. Luna, H T (ASCP) Editor, The Blakston Division McGraw-Hill Book Company, New York; The Armed Forces Institute of Pathology Advanced Laboratory Methods in Histology and Pathology (1994) Ulreka V. Mikel, Editor, Armed Forces Institute of Pathology, American Registry of Pathology, Washington, D.C. One of skill in the art will appreciate that the choice of a fixative is determined by the purpose for which the tissue is to be histologically stained or otherwise analyzed. One of skill in the art will also appreciate that the length of fixation depends upon the size of the tissue sample and the fixative used. By way of example, neutral buffered formalin, Bouin's or paraformaldehyde, may be used to fix a tissue sample.

Generally, a tissue sample is first fixed and is then dehydrated through an ascending series of alcohols, infiltrated and embedded with paraffin or other sectioning media so that the tissue sample may be sectioned. Alternatively, one may section the tissue and fix the sections obtained. By way of example, the tissue sample may be embedded and processed in paraffin by conventional methodology (See e.g., “Manual of Histological Staining Method of the Armed Forces Institute of Pathology”, supra). Examples of paraffin that may be used include, but are not limited to, Paraplast, Broloid, and Tissuemay. Once the tissue sample is embedded, the sample may be sectioned by a microtome or the like (See e.g., “Manual of Histological Staining Method of the Armed Forces Institute of Pathology”, supra). By way of example for this procedure, sections may have a thickness in a range from about three microns to about twelve microns, and preferably, a thickness in a range of from about 5 microns to about 10 microns. In one aspect, a section may have an area of from about 10 mm² to about 1 cm². Once cut, the sections may be attached to slides by several standard methods. Examples of slide adhesives include, but are not limited to, silane, gelatin, poly-L-lysine and the like. By way of example, the paraffin embedded sections may be attached to positively charged slides and/or slides coated with poly-L-lysine.

If paraffin has been used as the embedding material, the tissue sections are generally deparaffinized and rehydrated to water. The tissue sections may be deparaffinized by several conventional standard methodologies. For example, xylenes and a gradually descending series of alcohols may be used (See e.g., “Manual of Histological Staining Method of the Armed Forces Institute of Pathology”, supra). Alternatively, commercially available deparaffinizing non-organic agents such as Hemo-De® (CMS, Houston, Tex.) may be used.

For mammalian tissue culture cells, fresh tissues, or like sources, samples may be prepared by conventional cell lysis techniques (e.g. 0.14 M NaCl, 1.5 mM MgCl₂, 10 mM Tris-Cl (pH 8.6), 0.5% Nonidet P40, and protease and/or phosphatase inhibitors as required). For fresh mammalian tissues, sample preparation may also include a tissue disaggregation step, e.g. crushing, mincing, grinding, sonication, or the like.

B. Magnetic Isolation of Cells.

In some applications, such as measuring dimers on rare metastatic cells from a patient's blood, an enrichment step may be carried out prior to conducting an assay for surface receptor dimer populations. Immunomagnetic isolation or enrichment may be carried out using a variety of techniques and materials known in the art, as disclosed in the following representative references that are incorporated by reference: Terstappen et al, U.S. Pat. No. 6,365,362; Terstappen et al, U.S. Pat. No. 5,646,001; Rohr et al, U.S. Pat. No. 5,998,224; Kausch et al, U.S. Pat. No. 5,665,582; Kresse et al, U.S. Pat. No. 6,048,515; Kausch et al, U.S. Pat. No. 5,508,164; Miltenyi et al, U.S. Pat. No. 5,691,208; Molday, U.S. Pat. No. 4,452,773; Kronick, U.S. Pat. No. 4,375,407; Radbruch et al, chapter 23, in Methods in Cell Biology, Vol, 42 (Academic Press, New York, 1994); Uhlen et al, Advances in Biomagnetic Separation (Eaton Publishing, Natick, 1994); Safarik et al, J. Chromatography B, 722: 33-53 (1999); Miltenyi et al, Cytometry, 11: 231-238 (1990); Nakamura et al, Biotechnol. Prog., 17: 1145-1155 (2001); Moreno et al, Urology, 58: 386-392 (2001); Racila et al, Proc. Natl. Acad. Sci., 95: 4589-4594 (1998); Zigeuner et al, J. Urology, 169: 701-705 (2003); Ghossein et al, Seminars in Surgical Oncology, 20: 304-311 (2001).

The preferred magnetic particles for use in carrying out this invention are particles that behave as colloids. Such particles are characterized by their sub-micron particle size, which is generally less than about 200 nanometers (nm) (0.20 microns), and their stability to gravitational separation from solution for extended periods of time. In addition to the many other advantages, this size range makes them essentially invisible to analytical techniques commonly applied to cell analysis. Particles within the range of 90-150 nm and having between 70-90% magnetic mass are contemplated for use in the present invention. Suitable magnetic particles are composed of a crystalline core of superparamagnetic material surrounded by molecules which are bonded, e.g., physically absorbed or covalently attached, to the magnetic core and which confer stabilizing colloidal properties. The coating material should preferably be applied in an amount effective to prevent non specific interactions between biological macromolecules found in the sample and the magnetic cores. Such biological macromolecules may include sialic acid residues on the surface of non-target cells, lectins, glyproteins and other membrane components. In addition, the material should contain as much magnetic mass/nanoparticle as possible. The size of the magnetic crystals comprising the core is sufficiently small that they do not contain a complete magnetic domain. The size of the nanoparticles is sufficiently small such that their Brownian energy exceeds their magnetic moment. As a consequence, North Pole, South Pole alignment and subsequent mutual attraction/repulsion of these colloidal magnetic particles does not appear to occur even in moderately strong magnetic fields, contributing to their solution stability. Finally, the magnetic particles should be separable in high magnetic gradient external field separators. That characteristic facilitates sample handling and provides economic advantages over the more complicated internal gradient columns loaded with ferromagnetic beads or steel wool. Magnetic particles having the above-described properties can be prepared by modification of base materials described in U.S. Pat. Nos. 4,795,698, 5,597,531 and 5,698,271, which patents are incorporated by reference.

### Assay Using Releasable Molecular Tags

Many advantages are provided by measuring intracellular complex populations using releasable molecular tags, including (1) separation of released molecular tags from an assay mixture provides greatly reduced background and a significant gain in sensitivity; and (2) the use of molecular tags that are specially designed for ease of separation and detection provides a convenient multiplexing capability so that multiple receptor complex components may be readily measured simultaneously in the same assay. Assays employing such tags can have a variety of forms and are disclosed in the following references: Singh et al, U.S. Pat. No. 6,627,400; U.S. patent publications Singh et al, 2002/0013126; and 2003/0170915, and Williams et al, 2002/0146726; and Chan-Hui et al, International patent publication WO 2004/011900, all of which are incorporated herein by reference. For example, a wide variety of separation techniques may be employed that can distinguish molecules based on one or more physical, chemical, or optical differences among molecules being separated including but not limited to electrophoretic mobility, molecular weight, shape, solubility, pKa, hydrophobicity, charge, charge/mass ratio, polarity, or the like. In one aspect, molecular tags in a plurality or set differ in electrophoretic mobility and optical detection characteristics and are separated by electrophoresis. In another aspect, molecular tags in a plurality or set may differ in molecular weight, shape, solubility, pKa, hydrophobicity, charge, polarity, and are separated by normal phase or reverse phase HPLC, ion exchange HPLC, capillary electrochromatography, mass spectroscopy, gas phase chromatography, or like technique.

Sets of molecular tags are provided that are separated into distinct bands or peaks by a separation technique after they are released from binding compounds. Identification and quantification of such peaks provides a measure or profile of the kinds and amounts of receptor dimers. Molecular tags within a set may be chemically diverse; however, for convenience, sets of molecular tags are usually chemically related. For example, they may all be peptides, or they may consist of different combinations of the same basic building blocks or monomers, or they may be synthesized using the same basic scaffold with different substituent groups for imparting different separation characteristics, as described more fully below. The number of molecular tags in a plurality may vary depending on several factors including the mode of separation employed, the labels used on the molecular tags for detection, the sensitivity of the binding moieties, the efficiency with which the cleavable linkages are cleaved, and the like. In one aspect, the number of molecular tags in a plurality for measuring populations of receptor dimers is in the range of from 2 to 10. In other aspects, the size of the plurality may be in the range of from 2 to 8, 2 to 6, 2 to 4, or 2 to 3.

Intracellular complexes may be detected in assays having homogeneous formats or a non-homogeneous, i.e. heterogeneous, formats. In a homogeneous format, no step is required to separate binding compounds specifically bound to target complexes from unbound binding compounds. In a preferred embodiment, homogeneous formats employ reagent pairs comprising (i) one or more binding compounds with releasable molecular tags and (ii) at least one cleaving probe that is capable of generating an active species that reacts with and releases molecular tags within an effective proximity of the cleaving probe.

Intracellular complexes may also be detected by assays employing a heterogeneous format. Heterogeneous techniques normally involve a separation step, where intracellular complexes having binding compounds specifically bound are separated from unbound binding compounds, and optionally, other sample components, such as proteins, membrane fragments, and the like. Separation can be achieved in a variety of ways, such as employing a reagent bound to a solid support that distinguishes between complex-bound and unbound binding compounds. The solid support may be a vessel wall, e.g., microtiter well plate well, capillary, plate, slide, beads, including magnetic beads, liposomes, or the like. The primary characteristics of the solid support are that it (1) permits segregation of the bound and unbound binding compounds and (2) does not interfere with the formation of the binding complex, or the other operations in the determination of intracellular complexes. Usually, in fixed samples, unbound binding compounds are removed simply by washing.

With detection using molecular tags in a heterogeneous format, after washing, a sample may be combined with a solvent into which the molecular tags are to be released. Depending on the nature of the cleavable bond and the method of cleavage, the solvent may include any additional reagents for the cleavage. Where reagents for cleavage are not required, the solvent conveniently may be a separation buffer, e.g. an electrophoretic separation medium. For example, where the cleavable linkage is photolabile or cleavable via an active species generated by a photosensitizer, the medium may be irradiated with light of appropriate wavelength to release the molecular tags into the buffer.

In either format, if the assay reaction conditions interfere with the separation technique employed, it may be necessary to remove, or exchange, the assay reaction buffer prior to cleavage and separation of the molecular tags. For example, in some embodiments, assay conditions include salt concentrations (e.g. required for specific binding) that degrade separation performance when molecular tags are separated on the basis of electrophoretic mobility. In such embodiments, an assay buffer is replaced by a separation buffer, or medium, prior to release and separation of the molecular tags.

Assays employing releasable molecular tags and cleaving probes can be made in many different formats and configuations depending on the complexes that are detected or measured. Based on the present disclosure, it is a design choice for one of ordinary skill in the art to select the numbers and specificities of particular binding compounds and cleaving probes.

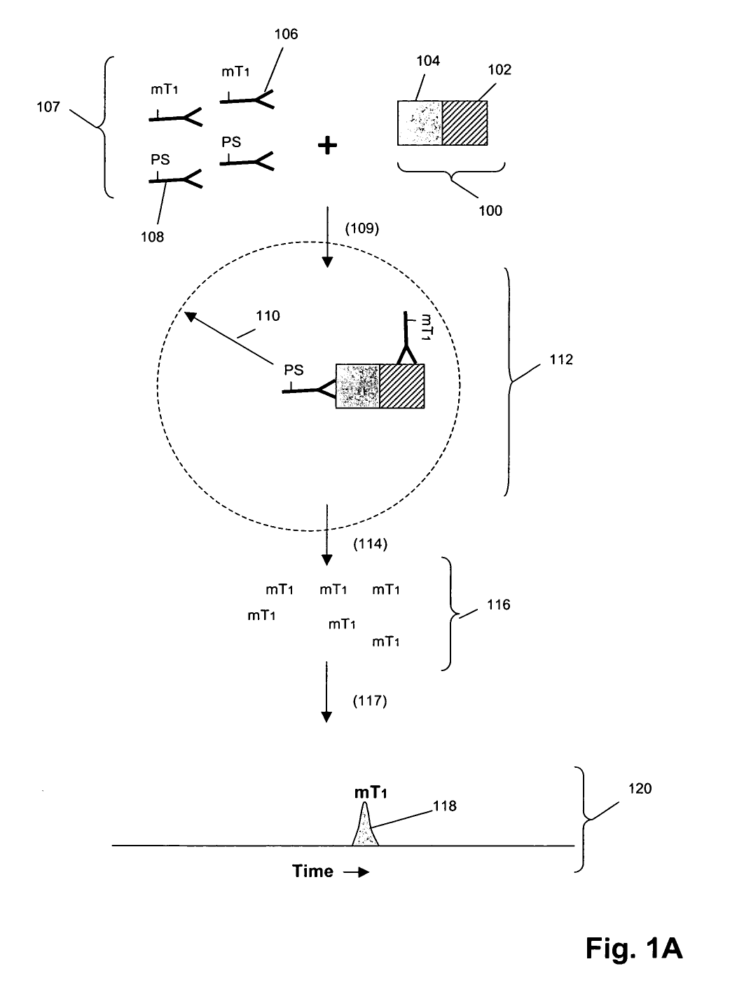

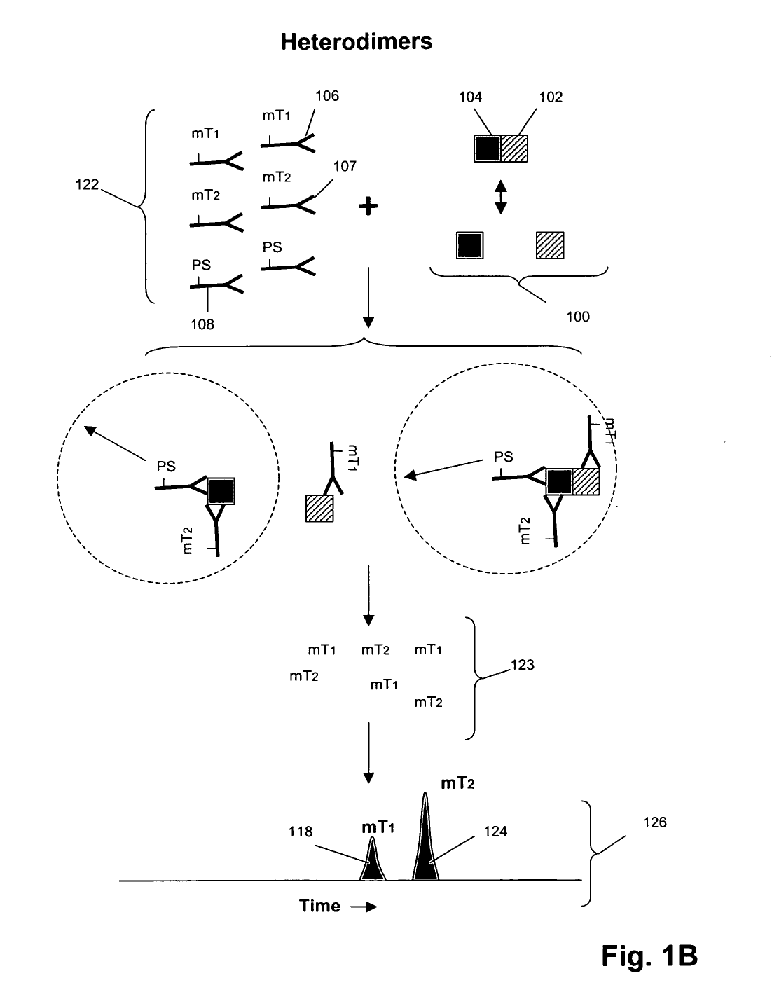

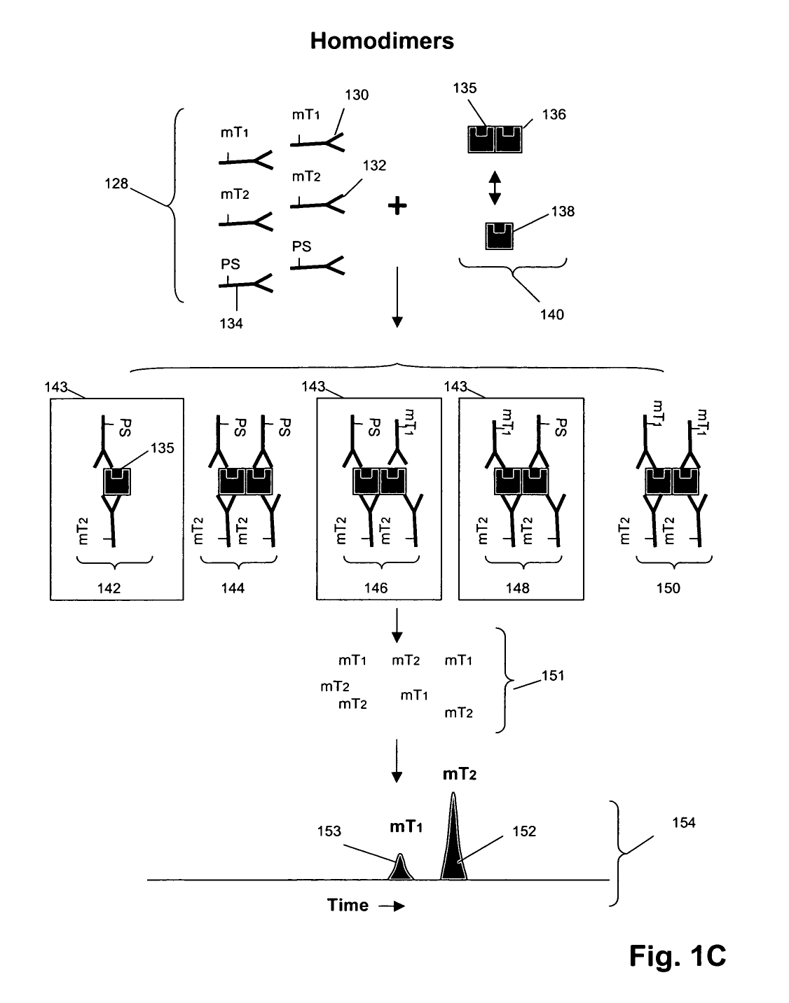

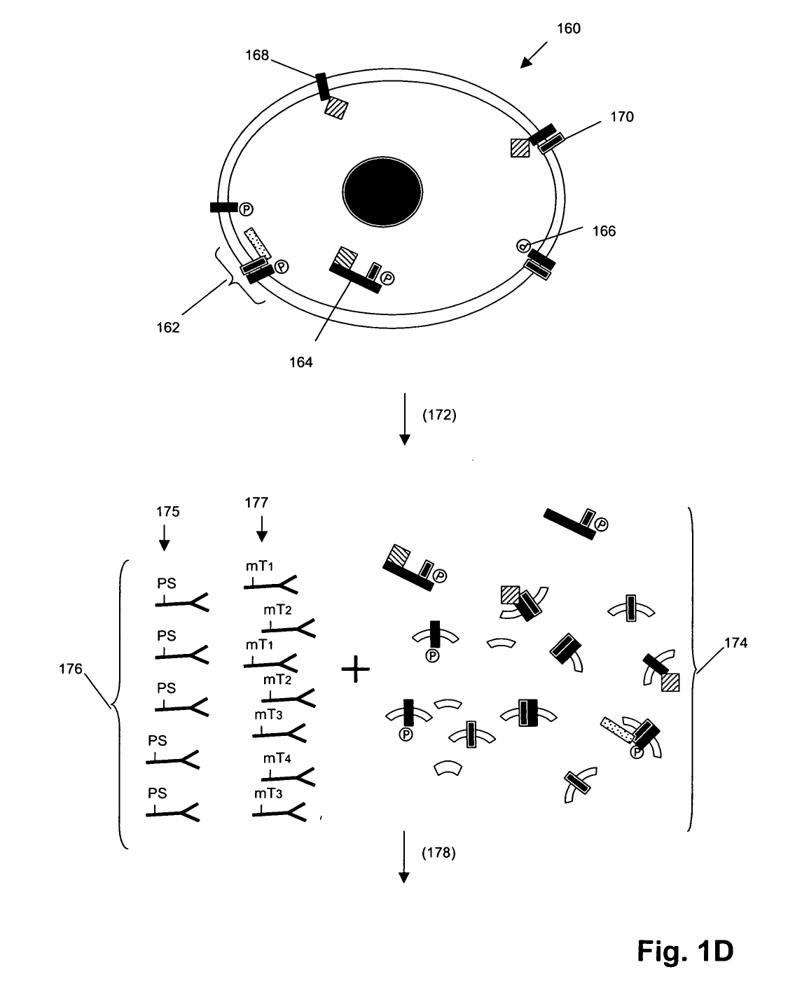

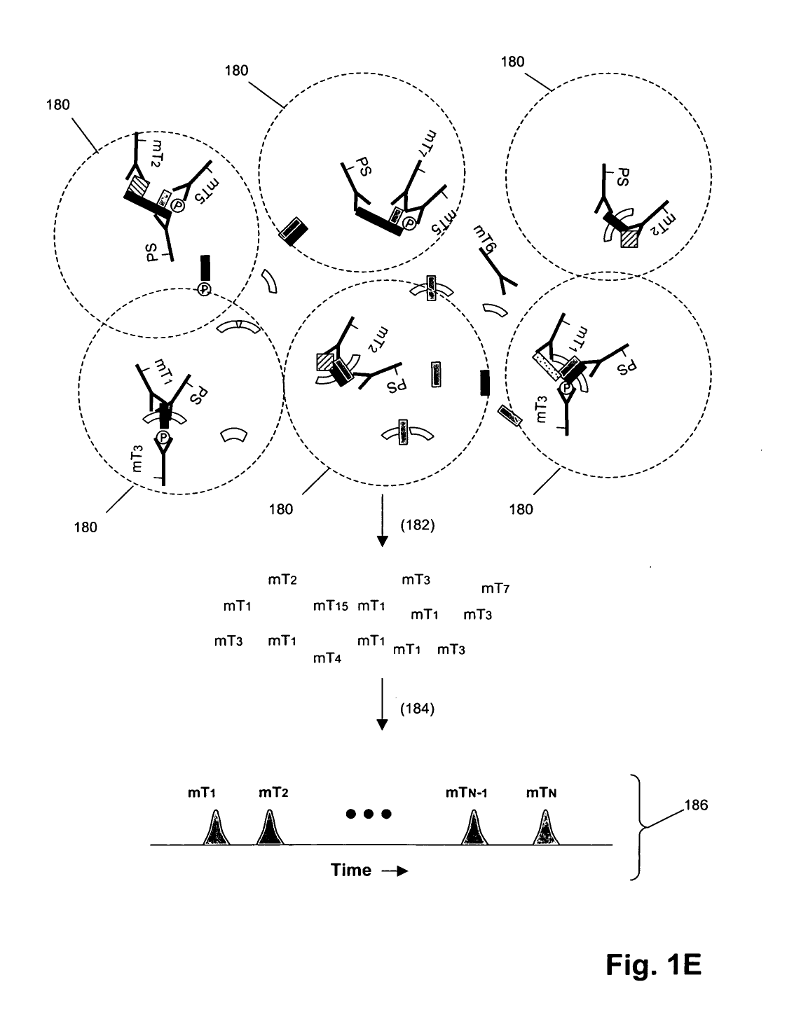

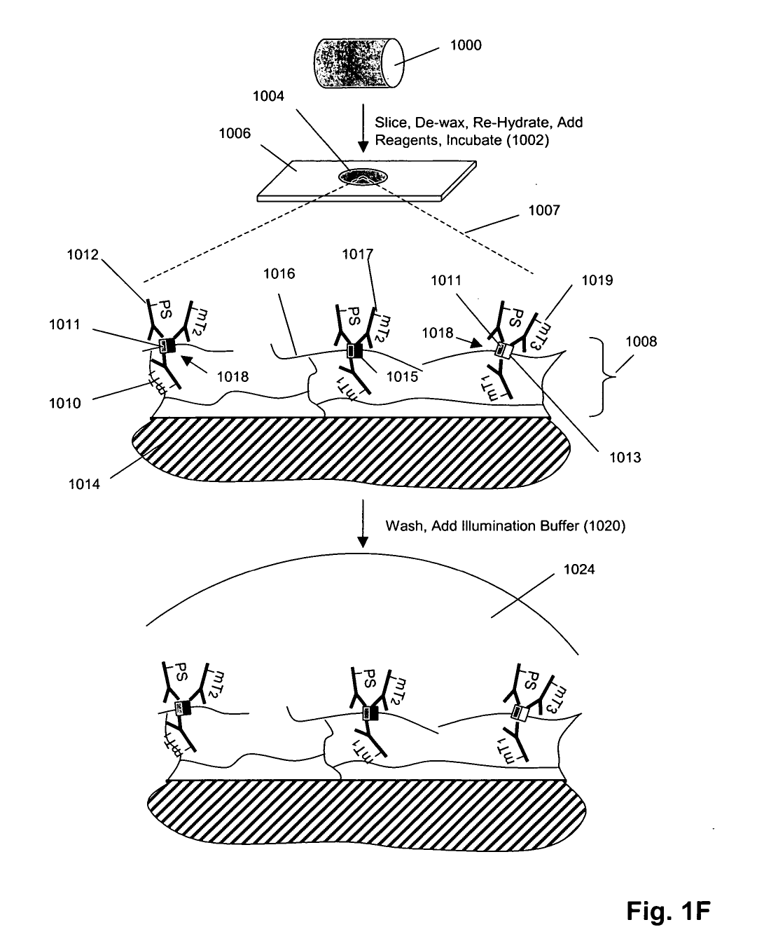

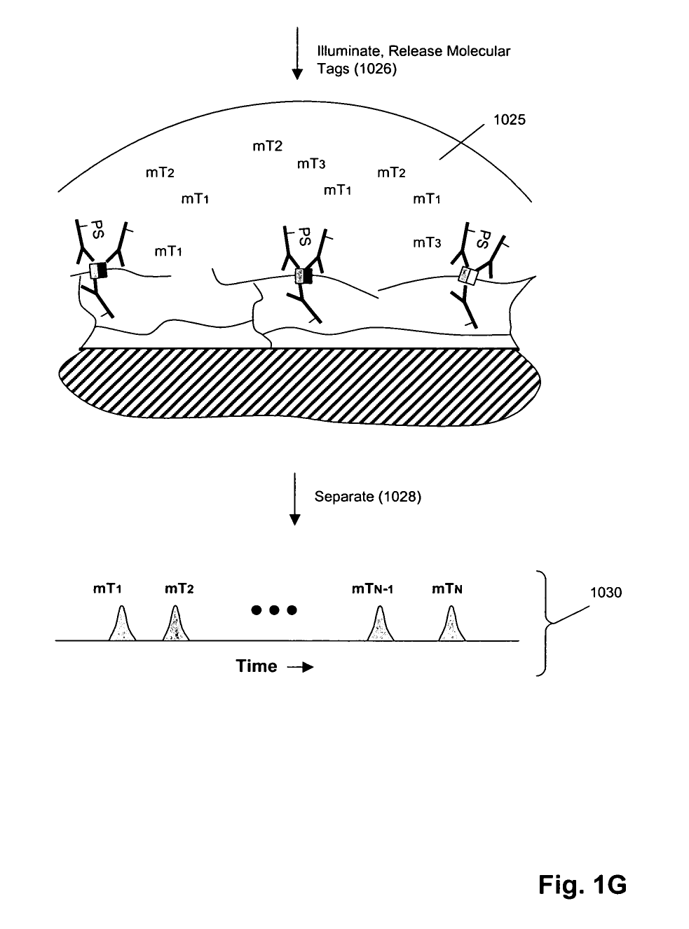

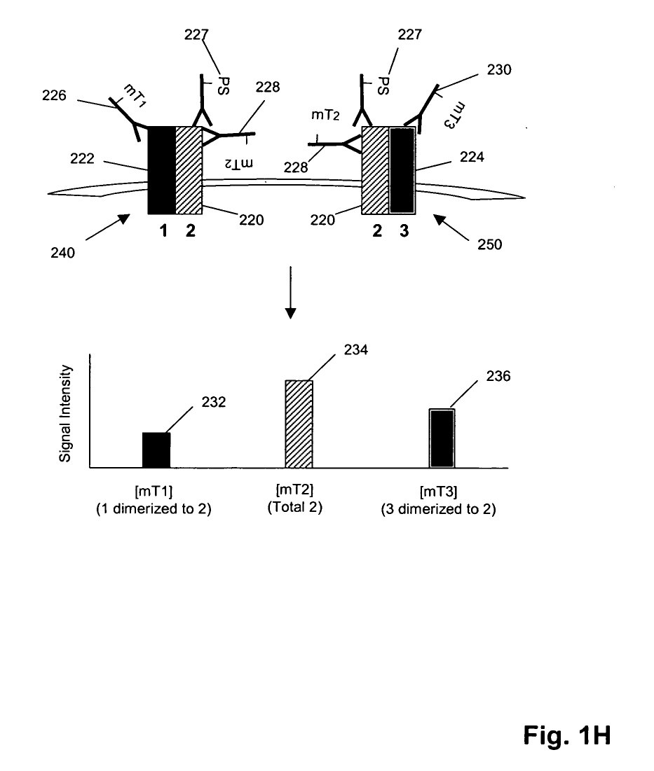

The operation of one embodiment of the invention is illustrated in FIG. 1A. Molecular complex (100) forms by the binding of proteins (104) and (102), e.g. 14-3 -3 and phosphorylated BAD. Reagents (107) of the invention, comprising cleaving probes (108) (in this illustration having photosensitizer “PS” attached) and binding compounds (106), are mixed (109) with a sample containing complex (100) under conditions that permit the specific binding (112) of cleaving probes (108) and binding compounds (106) to their respective antigenic determinants on complex (100) that are on different proteins of the complex. After binding, and optionally washing or buffer exchange, cleaving probes (108) are activated to generate an active species that, e.g. in the case of singlet oxygen, diffuses out from a photosensitizers to an effective proximity (110). Cleavable linkages within this proximity are cleaved and molecular tags are released (114). Released molecular tags (116) are then separated (117) and a separation profile (120), such as an electropherogram, is produced, in which peak (118) is identified and correlated to molecular tag, “mT₁.” By employing additional binding compounds and molecular tags, additional complexes may be measured. A more complex embodiment is illustrated in FIG. 1B, in which an additional binding compound is employed to give a measure of the total amount of protein (104) in a sample. Reagents (122) of the invention comprise (i) cleaving probes (108), first binding compound (106), and second binding compound (107), wherein first binding compound (106) is specific for protein (102) and second binding compound (107) is specific for protein (104) at a different antigenic determinant than that cleaving probe (108) is specific for. As with the embodiment of FIG. 1A, after binding of the reagents, cleaving probe (108) is activated to produce active species that cleave the cleavable linkages of the molecular tags within the effective proximity of the photosensitizer. In this embodiment, molecular tags are released from monomers of protein (104) that have both reagents (107) and (108) attached and from heterodimers that have reagent (108) attached and either or both of reagents (106) and (107) attached. Released molecular tags (123) are separated, and peaks (118 and 124) in a separation profile (126) are correlated to the amounts of the released molecular tags. In this embodiment, relative peak heights, or areas, may reflect (i) the differences in affinity of the first and second binding compounds for their respective antigenic determinants, and/or (ii) the presence or absense of the antigenic determinant that the binding compound is specific for. The later situation is important whenever a binding compound is used to monitor the post-translational state of a protein, e.g. phosphorylation state.

Homodimeric as well as heterodimeric complexes of apoptotic signaling molecules may be measured as illustrated in FIG. 1A-1C. FIG. 1C illustrates one approach for measuring homodimeric complexes. As above, an assay may comprise three reagents (128): cleaving probes (134), first binding compound (130), and second binding compound (132). First binding compound (130) and cleaving probe (134) are constructed to be specific for the same antigenic determinant (135) on protein (138) that exists (140) in a sample as either a homodimer (136) or a monomer (138). After reagents (128) are combined with a sample under conditions that promote the formation of stable complexes between the reagents and their respective targets, multiple complexes (142 through 150) form in the assay mixture. Because cleaving probe (134) and binding compound (130) are specific for the same antigenic determinant (135), four different combinations (144 throught 150) of reagents may form complexes with homodimers. Of the complexes in the assay mixture, only those (143) with both a cleaving probe (134) and at least one binding compound will contribute released molecular tags (151) for separation and detection (154). In this embodiment, the size of peak.(153) is proportional to the amount of homodimer in the assay mixture, while the size of peak (152) is proportional to the total amount of protein (138) in the assay mixture, both in monomeric form (142) or in homodimeric form (146 and 148).

Another aspect of the invention is illustrated in FIGS. 1D and 1E, which provides for the simultaneous detection or measurement of multiple complexes in a cellular sample. Cells (160), which may be from a sample from in vitro cultures or from a specimen of patient tissue, are lysed (172) to render accessable molecular complexes associated with the cell membrane, and/or within the cytosol, and/or within the cell nucleus. Complexes associated with apoptotic signaling include, but are not limited to, surface receptor complexes, such as receptor dimers, receptor complexes including adaptor or scaffold molecules of various types, dimers and higher order complexes of intracellular proteins, phosphorylation sites of proteins in such complexes, and the like. After lysing, the resulting lysate (174) is combined with assay reagents (176) that include multiple cleaving probes (175) and multiple binding compounds (177). Assay conditions are selected (178) that allow reagents (176) to specifically bind to their respective targets, so that upon activation cleavable linkages within the effective proximity (180) of the cleavage-inducing moieties are cleaved and molecular tags are released (182). As above, after cleavage, the released molecular tags are separated (184) and identified in a separation profile (186), such as an electropherogram, and based on the number and quantities of molecular tags measured, a profile is obtained of the selected molecular complexes in the cells of the sample.

FIGS. 1F and 1G illustrate an embodiment of the invention for measuring receptor complexes in fixed or frozen tissue samples. Fixed tissue sample (1000), e.g. a formalin-fixed paraffin-embedded sample, is sliced to provide a section (1004) using a microtome, or like instrument, which after placing on surface (1006), which may be a microscope slide, it is de-waxed and re-hydrated for application of assay reagents. Enlargement (1007) shows portion (1008) of section (1004) on portion (1014) of microscope slide (1006). Intracellular complexes (1018) are illustrated along with the remnants of membrane structure (1016) of the fixed sample. In accordance with this aspect of the invention, cleaving probe and binding compounds are incubated with the fixed sample so that they bind to their target molecules. For example, cleaving probes (1012)(illustrated in the figure as an antibody having a photosensitizer (“PS”) attached) and first binding compound (110)(illustrated as an antibody having molecular tag “mT₁” attached) specifically bind to protein (1011) common to all of the complexes shown, second binding compound (1017)(with “mT₂”) specifically binds to protein (1015), and third binding compound (1019)(with “mT₃”) specifically binds to protein (1013). After washing to remove binding compounds and cleaving probe that are not specifically bound to their respective target molecules, buffer (1024) (referred to as “illumination buffer” in the figure) is added. For convenience, buffer (1024) may be contained on section (1004), or a portion thereof, by creating a hydrophobic barrier on slide (1006), e.g. with a wax pen. After illumination of photosensitizers and release of molecular tags (1026), buffer (1024) now containing release molecular tags (1025) is transferred to a separation device, such as a capillary electrophoresis instrument, for separation (1028) and identification of the released molecular tags in, for example, electropherogram (1030).

Measurements made directly on tissue samples, particularly as illustrated in FIGS. 1F and 1G, may be normalized by including measurements on cellular or tissue targets that are representative of the total cell number in the sample and/or the numbers of particular subtypes of cells in the sample. Such tissue targets are referred to herein as “tissue indicators.” The additional measurement may be preferred, or even necessary, because of the cellular and tissue heterogeneity in patient samples, particularly tumor samples, which may comprise substantial fractions of normal cells. For example, in FIG. 1H, values for the total amount of receptor (1011) may be given as a ratio of the following two measurements: area of peak (1032) of molecular tag (“mT₁”) and the area of a peak corresponding to a molecular tag correlated with a cellular or tissue component common to all the cells in the sample, e.g. tubulin, or the like. In some cases, where all the cells in the sample are epithelial cells, or where the cells of interest are epithelial cells invading a non-epithelial tissue, cytokeratin, or cytokeratin and tubulin, may be used. Accordingly, detection methods based on releasable molecular tags may include an additional step of providing a binding compound (with a distinct molecular tag) specific for a normalization protein, such as tubulin.

A preferred embodiment for measuring relative amounts of receptor dimers containing a common component receptor is illustrated in FIG. 1H. In this assay design, two different receptor dimers (“1-2” (240) and “2-3” (250)) each having a common component, “2,” may be measured ratiometrically with respect to the common component. An assay design is shown for measuring receptor heterodimer (240) comprising receptor “1” (222) and receptor “2” (220) and receptor heterodimer (250) comprising receptor “2” (220) and receptor “3”(224). A key feature of this embodiment is that cleaving probe (227) be made specific for the common receptor of the pair of heterodimers. Binding compound (228) specific for receptor “2” provides a signal (234) related to the total amount of receptor “2” in the assay, whereas binding compound (226) specific for receptor “1” and binding compound (230) specific for receptor “3” provide signals (232 and 236, respectively) related only to the amount of receptor “1” and receptor “3” present as heterodimers with receptor “2,” respectively. The design of FIG. 1H may be generalized to more than two receptor complexes that contain a common component by simply adding binding compounds specific for the components of the additional complexes.

A. Binding Compounds

As mentioned above, an aspect of the invention includes providing mixtures of pluralities of different binding compounds, wherein each different binding compound has one or more molecular tags attached through cleavable linkages. The nature of the binding compound, cleavable linkage and molecular tag may vary widely. A binding compound may comprise an antibody binding composition, an antibody, a peptide, a peptide or non-peptide ligand for a cell surface receptor, a protein, an oligonucleotide, an oligonucleotide analog, such as a peptide nucleic acid, a lectin, or any other molecular entity that is capable of specific binding or stable complex formation with an analyte of interest, such as a complex of proteins. In one aspect, a binding compound, which can be represented by the formula below, comprises one or more molecular tags attached to a binding moiety.

B-(L-E)ₖ

wherein B is binding moiety; L is a cleavable linkage; and E is a molecular tag. In homogeneous assays, cleavable linkage, L, may be an oxidation-labile linkage, and more preferably, it is a linkage that may be cleaved by singlet oxygen. The moiety “-(L-E)ₖ” indicates that a single binding compound may have multiple molecular tags attached via cleavable linkages. In one aspect, k is an integer greater than or equal to one, but in other embodiments, k may be greater than several hundred, e.g. 100 to 500, or k is greater than several hundred to as many as several thousand, e.g. 500 to 5000. Usually each of the plurality of different types of binding compound has a different molecular tag, E. Cleavable linkages, e.g. oxidation-labile linkages, and molecular tags, E, are attached to B by way of conventional chemistries.

Preferably, B is an antibody binding composition. Such compositions are readily formed from a wide variety of commercially available antibodies, both monoclonal and polyclonal, specific for proteins of interest. In particular, antibodies specific for epidermal growth factor receptors are disclosed in the following patents, which are incorporated by references: U.S. Pat. Nos. 5,677,171; 5,772,997; 5,968,511; 5,480,968; 5,811,098. U.S. Pat. No. 6,488,390, incorporated herein by reference, discloses antibodies specific for a G-protein coupled receptor, CCR4. U.S. Pat. No. 5,599,681, incorporated herein by reference, discloses antibodies specific for phosphorylation sites of proteins. Commercial vendors, such as Cell Signaling Technology (Beverly, Mass.), Biosource International (Camarillo, Calif.), and Upstate (Charlottesville, Va.), also provide monoclonal and polyclonal antibodies specific for many proteins, e.g. proteins in apoptotic pathways, including proteins listed in Table II.

Cleavable linkage, L, can be virtually any chemical linking group that may be cleaved under conditions that do not degrade the structure or affect detection characteristics of the released molecular tag, E. Whenever a cleaving probe is used in a homogeneous assay format, cleavable linkage, L, is cleaved by a cleavage agent generated by the cleaving probe that acts over a short distance so that only cleavable linkages in the immediate proximity of the cleaving probe are cleaved. Typically, such an agent must be activated by making a physical or chemical change to the reaction mixture so that the agent produces a short lived active species that diffuses to a cleavable linkage to effect cleavage. In a homogeneous format, the cleavage agent is preferably attached to a binding moiety, such as an antibody, that targets prior to activation the cleavage agent to a particular site in the proximity of a binding compound with releasable molecular tags. In such embodiments, a cleavage agent is referred to herein as a “cleavage-inducing moiety,” which is discussed more fully below.

In a non-homogeneous format, because specifically bound binding compounds are separated from unbound binding compounds, a wider selection of cleavable linkages and cleavage agents are available for use. Cleavable linkages may not only include linkages that are labile to reaction with a locally acting reactive species, such as hydrogen peroxide, singlet oxygen, or the like, but also linkages that are labile to agents that operate throughout a reaction mixture, such as base-labile linkages, photocleavable linkages, linkages cleavable by reduction, linkages cleaved by oxidation, acid-labile linkages, peptide linkages cleavable by specific proteases, and the like. References describing many such linkages include Greene and Wuts, Protective Groups in Organic Synthesis, Second Edition (John Wiley & Sons, New York, 1991); Hermanson, Bioconjugate Techniques (Academic Press, New York, 1996); and Still et al, U.S. Pat. No. 5,565,324. Exemplary cleavable linkages are illustrated in Table III.

TABLE III

| Linking Group | Cleavage Reagent |
|---|---|
| silyl | fluoride or acid |
| A | hv |
| B | Ce(NH₄)₂(NO₃)₆ |
| —NCO₂— | HO⁻, H⁺, or LiAlH₄ |
| C | O₃, OsO₄/IO₄⁻, or KMnO₄ |
| D | 1) O₂ or Br₂, MeOH |
| | 2) H₃O⁺ |
| —Si— | oxidation, H⁺, Br₂, Cl₂, etc. |
| E | H₃O⁺ |
| F | H₃O⁺ |
| G | F⁻ or H⁺ |
| H, where x is a keto, ester, amide, | base, HO⁻ |
| NO₂, sulfide, sulfoxide, sulfone, | |
| and related electron withdrawing | |
| groups. | |
| I | H₃O⁺ or reduction (e.g. Li/NH₃) |
| J | (Ph₃P)₃RhCl(H) |
| K | Li, Mg, or BuLi |
| M | Hg⁺² |
| N, where x is halogen or | Zn or Mg |
| pseudohalogen | |
| O | oxidation (e.g. Pb(OAc)₄ or H₃IO₆) |
| P, where X is a electron | base |
| withdrawing group | |

Illustrative cleavable linking groups and cleavage reagents (L) shows the point of attachment of the molecular tag (E).

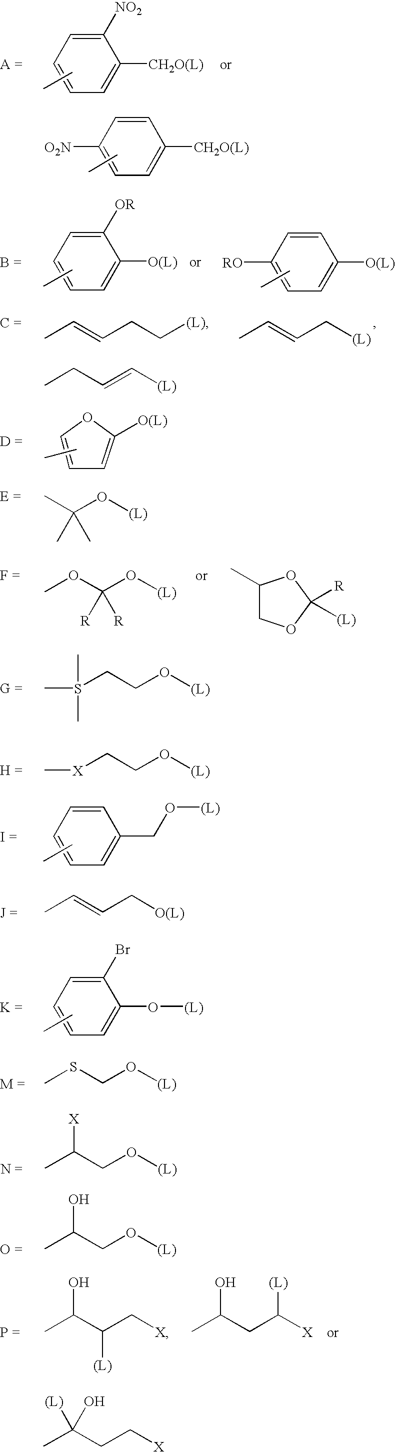

In one aspect, commercially available cleavable reagent systems may be employed with the invention. For example, a disulfide linkage may be introduced between an antibody binding composition and a molecular tag using a heterofunctional agent such as N-succinimidyl 3-(2-pyridyldithio)propionate (SPDP), succinimidyloxycarbonyl-α-methyl-α-(2-pyridyldithio)toluene (SMPT), or the like, available from vendors such as Pierce Chemical Company (Rockford, Ill.). Disulfide bonds introduced by such linkages can be broken by treatment with a reducing agent, such as dithiothreitol (DTT), dithioerythritol (DTE), 2-mercaptoethanol, sodium borohydride, or the like. Typical concentrations of reducing agents to effect cleavage of disulfide bonds are in the range of from 10 to 100 mM. An oxidatively labile linkage may be introduced between an antibody binding composition and a molecular tag using the homobifunctional NHS ester cross-linking reagent, disuccinimidyl tartarate (DST)(available from Pierce) that contains central cis-diols that are susceptible to cleavage with sodium periodate (e.g., 15 mM periodate at physiological pH for 4 hours). Linkages that contain esterified spacer components may be cleaved with strong nucleophilic agents, such as hydroxylamine, e.g. 0.1 N hydroxylamine, pH 8.5, for 3-6 hours at 37° C. Such spacers can be introduced by a homobifunctional cross-linking agent such as ethylene glycol bis(succinimidylsuccinate)(EGS) available from Pierce (Rockford, Ill.). A base labile linkage can be introduced with a sulfone group. Homobifunctional cross-linking agents that can be used to introduce sulfone groups in a cleavable linkage include bis[2-(succinimidyloxycarbonyloxy)ethyl]sulfone (BSOCOES), and 4,4-difluoro-3,3 -dinitrophenylsulfone (DFDNPS). Exemplary basic conditions for cleavage include 0.1 M sodium phosphate, adjusted to pH 11.6 by addition of Tris base, containing 6 M urea, 0.1% SDS, and 2 mM DTT, with incubation at 37° C. for 2 hours. Photocleavable linkages include those disclosed in Rothschild et al, U.S. Pat. No. 5,986,076.

When L is oxidation labile, L may be a thioether or its selenium analog; or an olefin, which contains carbon-carbon double bonds, wherein cleavage of a double bond to an oxo group, releases the molecular tag, E. Illustrative thioether bonds are disclosed in Willner et al, U.S. Pat. No. 5,622,929 which is incorporated by reference. Illustrative olefins include vinyl sulfides, vinyl ethers, enamines, imines substituted at the carbon atoms with an α-methine (CH, a carbon atom having at least one hydrogen atom), where the vinyl group may be in a ring, the heteroatom may be in a ring, or substituted on the cyclic olefinic carbon atom, and there will be at least one and up to four heteroatoms bonded to the olefinic carbon atoms. The resulting dioxetane may decompose spontaneously, by heating above ambient temperature, usually below about 75° C., by reaction with acid or base, or by photo-activation in the absence or presence of a photosensitizer. Such linkages and reactions are described in the following exemplary references: U.S. Pat. Nos. 5,756,726; 5,800,999; and 5,886,238.

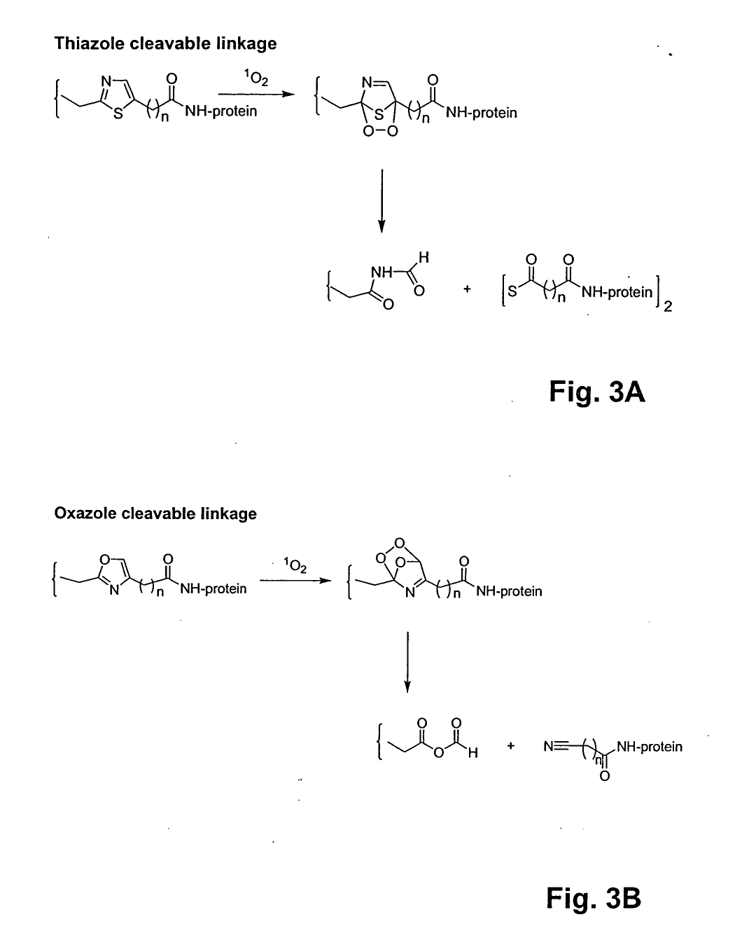

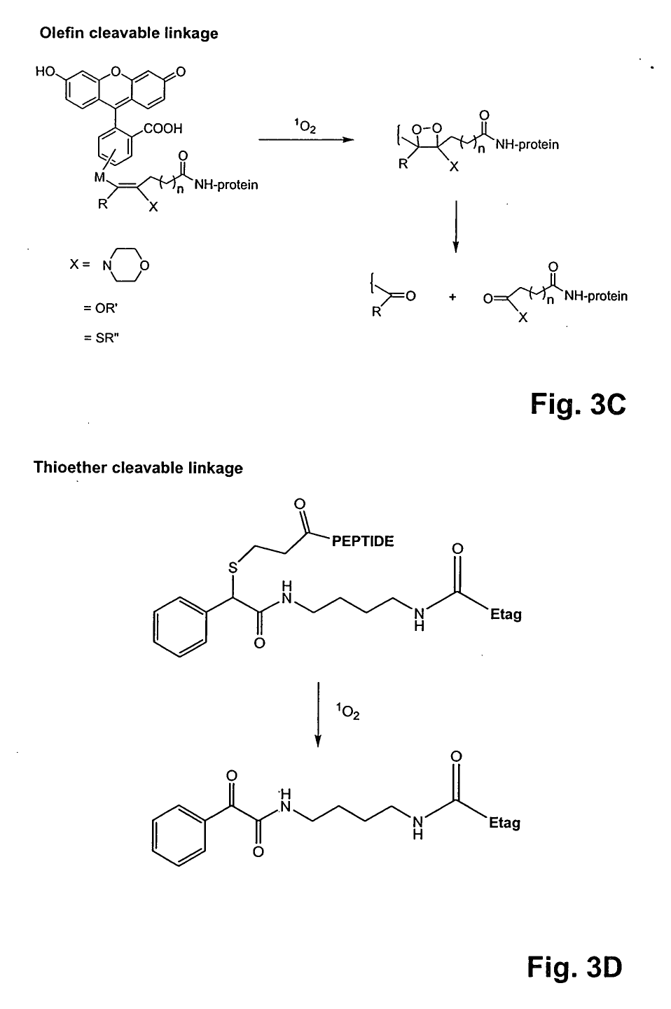

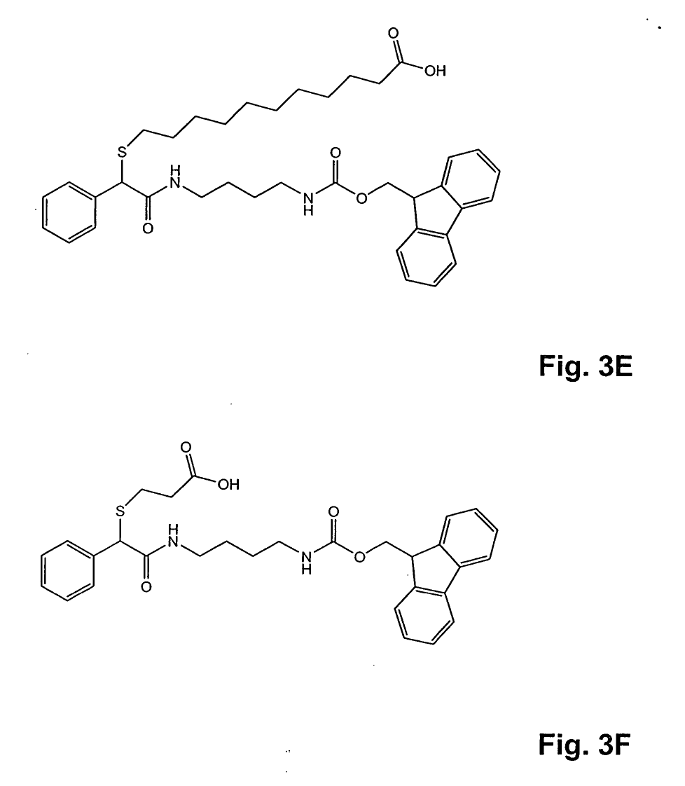

Exemplary cleavable linkages and their cleavage products are illustrated in FIGS. 3A-F. The thiazole cleavable linkage, “—CH₂-thiazole-(CH2)ₙ—C(═O)—NH-protein,” shown in FIG. 3A, results in an molecular tag with the moiety “—CH₂—C(═O)—NH—CHO.” Preferably, n is in the range of from 1 to 12, and more preferably, from 1 to 6. The oxazole cleavable linkage, “—CH₂-oxazole-(CH2)ₙ—C(═O)—NH-protein,” shown in FIG. 3B, results in an molecular tag with the moiety “—CH₂—C(═O)O—CHO.” An olefin cleavable linkage (FIG. 3C) is shown in connection with the binding compound embodiment “B-L-M-D,” described above and with D being a detection moiety, such as a fluorescein dye. The olefin cleavable linkage may be employed in other embodiments also. Cleavage of the illustrated olefin linkage results in an molecular tag of the form: “R—(C═O)—M—D,” where “R” may be any substituent within the general description of the molecular tags, E, provided above. Preferably, R is an electron-donating group, e.g. Ullman et al, U.S. Pat. No. 6,251,581; Smith and March, March's Advanced Organic Chemistry: Reactions, Mechanisms, and Structure, 5^{th} Edition (Wiley-Interscience, New York, 2001); and the like. More preferably, R is an electron-donating group having from 1-8 carbon atoms and from 0 to 4 heteroatoms selected from the group consisting of O, S, and N. In further preference, R is —N(Q)₂, —OQ, p-[C₆H₄N(Q)₂], furanyl, n-alkylpyrrolyl, 2-indolyl, or the like, where Q is alkyl or aryl. In further reference to the olefin cleavable linkage of FIG. 3C, substituents “X” and “R” are equivalent to substituents “X” and “Y” of the above formula describing cleavable linkage, L. In particular, X in FIG. 3C is preferably morpholino, —OR′, or —SR″, where R′ and R″ are aliphatic, aromatic, alicyclic or heterocyclic having from 1 to 8 carbon atoms and 0 to 4 heteroatoms selected from the group consisting of O, S. and N. A preferred thioether cleavable linkage is illustrated in FIG. 3D having the form “—(CH₂)₂—S—CH(C₆H₅)C(═O)NH—(CH₂)ₙ—NH—,” wherein n is in the range of from 2 to 12, and more preferably, in the range of from 2 to 6. Thioether cleavable linkages of the type shown in FIG. 3D may be attached to binding moieties, T, and molecular tags, E, by way of precursor compounds shown in FIGS. 3E and 3F. To attach to an amino group of a binding moiety, T, the terminal hydroxyl is converted to an NHS ester by conventional chemistry. After reaction with the amino group and attachment, the Fmoc protection group is removed to produce a free amine which is then reacted with an NHS ester of the molecular tag.

Molecular tag, E, in the present invention may comprise an electrophoric tag as described in the following references when separation of pluralities of molecular tags are carried out by gas chromatography or mass spectrometry: Zhang et al, Bioconjugate Chem., 13: 1002-1012 (2002); Giese, Anal. Chem., 2: 165-168 (1983); and U.S. Pat. Nos. 4,650,750; 5,360,819; 5,516,931; 5,602,273; and the like.

Molecular tag, E, is preferably a water-soluble organic compound that is stable with respect to the active species, especially singlet oxygen, and that includes a detection or reporter group. Otherwise, E may vary widely in size and structure. In one aspect, E has a molecular weight in the range of from about 50 to about 2500 daltons, more preferably, from about 50 to about 1500 daltons. Preferred structures of E are described more fully below. E may comprise a detection group for generating an electrochemical, fluorescent, or chromogenic signal. In embodiments employing detection by mass, E may not have a separate moiety for detection purposes. Preferably, the detection group generates a fluorescent signal.

Molecular tags within a plurality are selected so that each has a unique separation characteristic and/or a unique optical property with respect to the other members of the same plurality. In one aspect, the chromatographic or electrophoretic separation characteristic is retention time under set of standard separation conditions conventional in the art, e.g. voltage, column pressure, column type, mobile phase, electrophoretic separation medium, or the like. In another aspect, the optical property is a fluorescence property, such as emission spectrum, fluorescence lifetime, fluorescence intensity at a given wavelength or band of wavelengths, or the like. Preferably, the fluorescence property is fluorescence intensity. For example, each molecular tag of a plurality may have the same fluorescent emission properties, but each will differ from one another by virtue of a unique retention time. On the other hand, or two or more of the molecular tags of a plurality may have identical migration, or retention, times, but they will have unique fluorescent properties, e.g. spectrally resolvable emission spectra, so that all the members of the plurality are distinguishable by the combination of molecular separation and fluorescence measurement.

Preferably, released molecular tags are detected by electrophoretic separation and the fluorescence of a detection group. In such embodiments, molecular tags having substantially identical fluorescence properties have different electrophoretic mobilities so that distinct peaks in an electropherogram are formed under separation conditions. Preferably, pluralities of molecular tags of the invention are separated by conventional capillary electrophoresis apparatus, either in the presence or absence of a conventional sieving matrix. Exemplary capillary electrophoresis apparatus include Applied Biosystems (Foster City, Calif.) models 310, 3100 and 3700; Beckman (Fullerton, Calif.) model P/ACE MDQ; Amersham Biosciences (Sunnyvale, Calif.) MegaBACE 1000 or 4000; SpectruMedix genetic analysis system; and the like. Electrophoretic mobility is proportional to q/M^{2/3}, where q is the charge on the molecule and M is the mass of the molecule. Desirably, the difference in mobility under the conditions of the determination between the closest electrophoretic labels will be at least about 0.001, usually 0.002, more usually at least about 0.01, and may be 0.02 or more. Preferably, in such conventional apparatus, the electrophoretic mobilities of molecular tags of a plurality differ by at least one percent, and more preferably, by at least a percentage in the range of from 1 to 10 percent.

In one aspect, molecular tag, E, is (M, D), where M is a mobility-modifying moiety and D is a detection moiety. The notation “(M, D)” is used to indicate that the ordering of the M and D moieties may be such that either moiety can be adjacent to the cleavable linkage, L. That is, “B-L-(M, D)” designates binding compound of either of two forms: “B-L-M-D” or “B-L-D-M.”

Detection moiety, D, may be a fluorescent label or dye, a chromogenic label or dye, an electrochemical label, or the like. Preferably, D is a fluorescent dye. Exemplary fluorescent dyes for use with the invention include water-soluble rhodamine dyes, fluoresceins, 4,7-dichlorofluoresceins, benzoxanthene dyes, and energy transfer dyes, disclosed in the following references: Handbook of Molecular Probes and Research Reagents, 8^{th} ed., (Molecular Probes, Eugene, 2002); Lee et al, U.S. Pat. No. 6,191,278; Lee et al, U.S. Pat. No. 6,372,907; Menchen et al, U.S. Pat. No. 6,096,723; Lee et al, U.S. Pat. No. 5,945,526; Lee et al, Nucleic Acids Research, 25: 2816-2822 (1997); Hobb, Jr., U.S. Pat. No. 4,997,928; Khanna et al., U.S. Pat. No. 4,318,846; and the like. Preferably, D is a fluorescein or a fluorescein derivative.

The size and composition of mobility-modifying moiety, M, can vary from a bond to about 100 atoms in a chain, usually not more than about 60 atoms, more usually not more than about 30 atoms, where the atoms are carbon, oxygen, nitrogen, phosphorous, boron and sulfur. Generally, when other than a bond, the mobility-modifying moiety has from about 0 to about 40, more usually from about 0 to about 30 heteroatoms, which in addition to the heteroatoms indicated above may include halogen or other heteroatom. The total number of atoms other than hydrogen is generally fewer than about 200 atoms, usually fewer than about 100 atoms. Where acid groups are present, depending upon the pH of the medium in which the mobility-modifying moiety is present, various cations may be associated with the acid group. The acids may be organic or inorganic, including carboxyl, thionocarboxyl, thiocarboxyl, hydroxamic, phosphate, phosphite, phosphonate, phosphinate, sulfonate, sulfinate, boronic, nitric, nitrous, etc. For positive charges, substituents include amino (includes ammonium), phosphonium, sulfonium, oxonium, etc., where substituents are generally aliphatic of from about 1-6 carbon atoms, the total number of carbon atoms per heteroatom, usually be less than about 12, usually less than about 9. The side chains include amines, ammonium salts, hydroxyl groups, including phenolic groups, carboxyl groups, esters, amides, phosphates, heterocycles. M may be a homo-oligomer or a hetero-oligomer, having different monomers of the same or different chemical characteristics, e.g., nucleotides and amino acids.

B. Attaching Molecular Tags to Binding Moieties

Extensive guidance can be found in the literature for covalently linking molecular tags to binding compounds, such as antibodies, e.g. Hermanson, Bioconjugate Techniques, (Academic Press, New York, 1996), and the like. In one aspect of the invention, one or more molecular tags are attached directly or indirectly to common reactive groups on a binding compound. Common reactive groups include amine, thiol, carboxylate, hydroxyl, aldehyde, ketone, and the like, and may be coupled to molecular tags by commercially available cross-linking agents, e.g. Hermanson (cited above); Haugland, Handbook of Fluorescent Probes and Research Products, Ninth Edition (Molecular Probes, Eugene, Oreg., 2002). In one embodiment, an NHS-ester of a molecular tag is reacted with a free amine on the binding compound.

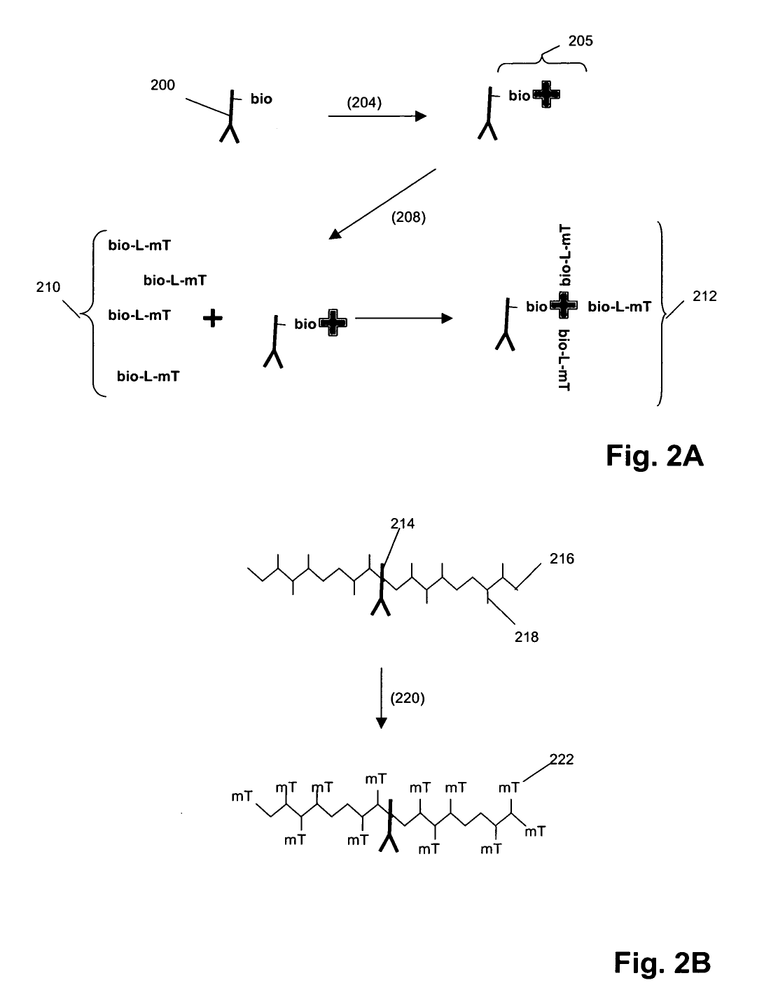

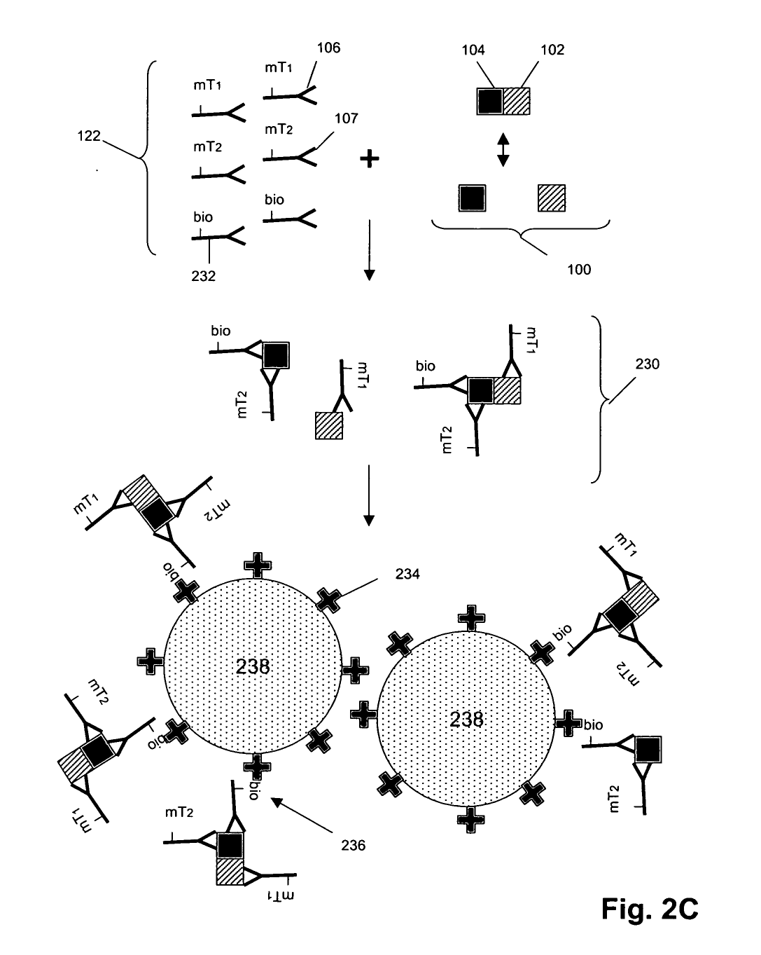

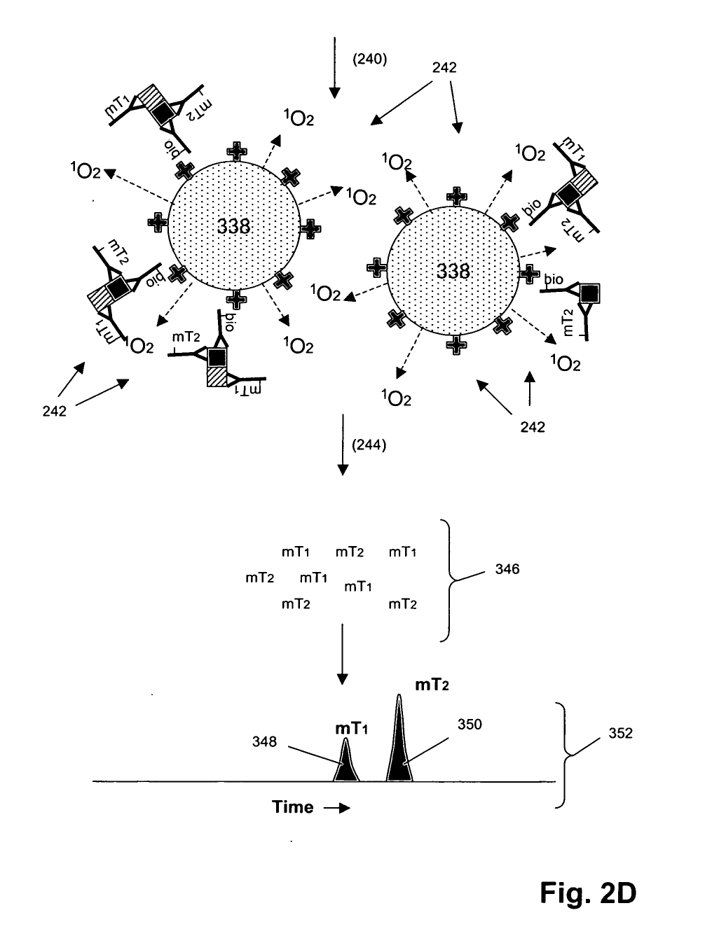

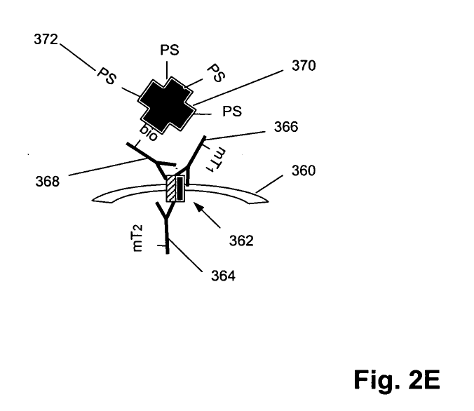

In another embodiment illustrated in FIG. 2A, binding compounds comprise a biotinylated antibody (200) as a binding moiety. Molecular tags are attached to binding moiety (200) by way of avidin or streptavidin bridge (206). Preferably, in operation, binding moiety (200) is first reacted with a target complex, after which avidin or streptavidin is added (204) to form antibody-biotin-avidin complex (205). To such complexes (205) are added (208) biotinylated molecular tags (210) to form binding compound (212).

In still another embodiment illustrated in FIG. 2B, binding compounds comprise an antibody (214) derivatized with a multi-functional moiety (216) that contains multiple functional groups (218) that are reacted (220) molecular tag precursors to give a final binding compound having multiple molecular tags (222) attached. Exemplary multi-functional moieties include aminodextran, and like materials.

Once each of the binding compounds is separately derivatized by a different molecular tag, it is pooled with other binding compounds to form a plurality of binding compounds. Usually, each different kind of binding compound is present in a composition in the same proportion; however, proportions may be varied as a design choice so that one or a subset of particular binding compounds are present in greater or lower proportion depending on the desirability or requirements for a particular embodiment or assay. Factors that may affect such design choices include, but are not limited to, antibody affinity and avidity for a particular target, relative prevalence of a target, fluorescent characteristics of a detection moiety of a molecular tag, and the like.

C. Cleavage-Inducing Moiety Producing Active Species

A cleavage-inducing moiety, or cleaving agent, is a group that produces an active species that is capable of cleaving a cleavable linkage, preferably by oxidation. Preferably, the active species is a chemical species that exhibits short-lived activity so that its cleavage-inducing effects are only in the proximity of the site of its generation. Either the active species is inherently short lived, so that it will not create significant background because beyond the proximity of its creation, or a scavenger is employed that efficiently scavenges the active species, so that it is not available to react with cleavable linkages beyond a short distance from the site of its generation. Illustrative active species include singlet oxygen, hydrogen peroxide, NADH, and hydroxyl radicals, phenoxy radical, superoxide, and the like. Illustrative quenchers for active species that cause oxidation include polyenes, carotenoids, vitamin E, vitamin C, amino acid-pyrrole N-conjugates of tyrosine, histidine, and glutathione, and the like, e.g. Beutner et al, Meth. Enzymol., 319: 226-241 (2000).

An important consideration for the cleavage-inducing moiety and the cleavable linkage is that they not be so far removed from one another when bound to a target protein that the active species generated by the sensitizer diffuses and loses its activity before it can interact with the cleavable linkage. Accordingly, a cleavable linkage preferably are within 1000 nm, preferably 20-200 nm of a bound cleavage-inducing moiety. This effective range of a cleavage-inducing moiety is referred to herein as its “effective proximity.”

Generators of active species include enzymes, such as oxidases, such as glucose oxidase, xanthene oxidase, D-amino acid oxidase, NADH-FMN oxidoreductase, galactose oxidase, glyceryl phosphate oxidase, sarcosine oxidase, choline oxidase and alcohol oxidase, that produce hydrogen peroxide, horse radish peroxidase, that produces hydroxyl radical, various dehydrogenases that produce NADH or NADPH, urease that produces ammonia to create a high local pH.

A sensitizer is a compound that can be induced to generate a reactive intermediate, or species, usually singlet oxygen. Preferably, a sensitizer used in accordance with the invention is a photosensitizer. Other sensitizers included within the scope of the invention are compounds that on excitation by heat, light, ionizing radiation, or chemical activation will release a molecule of singlet oxygen. The best known members of this class of compounds include the endoperoxides such as 1,4-biscarboxyethyl-1,4-naphthalene endoperoxide, 9,10-diphenylanthracene-9,10-endoperoxide and 5,6,11,12-tetraphenyl naphthalene 5,12-endoperoxide. Heating or direct absorption of light by these compounds releases singlet oxygen. Further sensitizers are disclosed in the following references: Di Mascio et al, FEBS Lett., 355: 287 (1994)(peroxidases and oxygenases); Kanofsky, J.Biol. Chem. 258: 5991-5993 (1983)(lactoperoxidase); Pierlot et al, Meth. Enzymol., 319: 3-20 (2000)(thermal lysis of endoperoxides); and the like.

Attachment of a binding agent to the cleavage-inducing moiety may be direct or indirect, covalent or non-covalent and can be accomplished by well-known techniques, commonly available in the literature. See, for example, “Immobilized Enzymes,” Ichiro Chibata, Halsted Press, New York (1978); Cuatrecasas, J. Biol. Chem., 245:3059 (1970). A wide variety of functional groups are available or can be incorporated. Functional groups include carboxylic acids, aldehydes, amino groups, cyano groups, ethylene groups, hydroxyl groups, mercapto groups, and the like. The manner of linking a wide variety of compounds is well known and is amply illustrated in the literature (see above). The length of a linking group to a binding agent may vary widely, depending upon the nature of the compound being linked, the effect of the distance on the specific binding properties and the like.

The cleavage-inducing moiety may be associated with the support by being covalently or non-covalently attached to the surface of the support or incorporated into the body of the support. Linking to the surface may be accomplished as discussed above. The cleavage-inducing moiety may be incorporated into the body of the support either during or after the preparation of the support. In general, the cleavage-inducing moiety is associated with the support in an amount necessary to achieve the necessary amount of active species. Generally, the amount of cleavage-inducing moiety is determined empirically.

As mentioned above, the preferred cleavage-inducing moiety in accordance with the present invention is a photosensitizer that produces singlet oxygen. As used herein, “photosensitizer” refers to a light-adsorbing molecule that when activated by light converts molecular oxygen into singlet oxygen. Photosensitizers may be attached directly or indirectly, via covalent or non-covalent linkages, to the binding agent of a class-specific reagent. Guidance for constructiing of such compositions, particularly for antibodies as binding agents, available in the literature, e.g. in the fields of photodynamic therapy, immunodiagnostics, and the like. The following are exemplary references: Ullman, et al., Proc. Natl. Acad. Sci. USA 91, 5426-5430 (1994); Strong et al, Ann. New York Acad. Sci., 745: 297-320 (1994); Yarmush et al, Crit. Rev. Therapeutic Drug Carrier Syst., 10: 197-252 (1993); Pease et al, U.S. Pat. No. 5,709,994; Ullman et al, U.S. Pat. No. 5,340,716; Ullman et al, U.S. Pat. No. 6,251,581; McCapra, U.S. Pat. No. 5,516,636; and the like.

Likewise, there is guidance in the literature regarding the properties and selection of photosensitizers suitable for use in the present invention. The following are exemplary references: Wasserman and R. W. Murray. Singlet Oxygen. (Academic Press, New York, 1979); Baumstark, Singlet Oxygen, Vol. 2 (CRC Press Inc., Boca Raton, Fla. 1983); and Turro, Modern Molecular Photochemistry (University Science Books, 1991).

The photosensitizers are sensitizers for generation of singlet oxygen by excitation with light. The photosensitizers include dyes and aromatic compounds, and are usually compounds comprised of covalently bonded atoms, usually with multiple conjugated double or triple bonds. The compounds typically absorb light in the wavelength range of about 200 to about 1,100 nm, usually, about 300 to about 1,000 nm, preferably, about 450 to about 950 nm, with an extinction coefficient at its absorbance maximum greater than about 500 M⁻¹ cm⁻¹, preferably, about 5,000 M⁻¹ cm⁻¹, more preferably, about 50,000 M⁻cm⁻¹, at the excitation wavelength. The lifetime of an excited state produced following absorption of light in the absence of oxygen will usually be at least about 100 nanoseconds, preferably, at least about 1 millisecond. In general, the lifetime must be sufficiently long to permit cleavage of a linkage in a reagent in accordance with the present invention. Such a reagent is normally present at concentrations as discussed below. The photosensitizer excited state usually has a different spin quantum number (S) than its ground state and is usually a triplet (S=1) when the ground state, as is usually the case, is a singlet (S=0). Preferably, the photosensitizer has a high intersystem crossing yield. That is, photoexcitation of a photosensitizer usually produces a triplet state with an efficiency of at least about 10%, desirably at least about 40%, preferably greater than about 80%.

Photosensitizers chosen are relatively photostable and, preferably, do not react efficiently with singlet oxygen. Several structural features are present in most useful photosensitizers. Most photosensitizers have at least one and frequently three or more conjugated double or triple bonds held in a rigid, frequently aromatic structure. They will frequently contain at least one group that accelerates intersystem crossing such as a carbonyl or imine group or a heavy atom selected from rows 3-6 of the periodic table, especially iodine or bromine, or they may have extended aromatic structures.

A large variety of light sources are available to photo-activate photosensitizers to generate singlet oxygen. Both polychromatic and monchromatic sources may be used as long as the source is sufficiently intense to produce enough singlet oxygen in a practical time duration. The length of the irradiation is dependent on the nature of the photosensitizer, the nature of the cleavable linkage, the power of the source of irradiation, and its distance from the sample, and so forth. In general, the period for irradiation may be less than about a microsecond to as long as about 10 minutes, usually in the range of about one millisecond to about 60 seconds. The intensity and length of irradiation should be sufficient to excite at least about 0.1% of the photosensitizer molecules, usually at least about 30% of the photosensitizer molecules and preferably, substantially all of the photosensitizer molecules. Exemplary light sources include, by way of illustration and not limitation, lasers such as, e.g., helium-neon lasers, argon lasers, YAG lasers, He/Cd lasers, and ruby lasers; photodiodes; mercury, sodium and xenon vapor lamps; incandescent lamps such as, e.g., tungsten and tungsten/halogen; flashlamps; and the like. By way of example, a photoactivation device disclosed in Bjornson et al, International patent publication WO 03/051669 is employed. Briefly, the photoactivation device is an array of light emitting diodes (LEDs) mounted in housing that permits the simultaneous illumination of all the wells in a 96-well plate. A suitable LED for use in the present invention is a high power GaAIAs IR emitter, such as model OD-880W manufactured by OPTO DIODE CORP. (Newbury Park, Calif.).

Examples of photosensitizers that may be utilized in the present invention are those that have the above properties and are enumerated in the following references: Singh and Ullman, U.S. Pat. No. 5,536,834; Li et al, U.S. Pat. No. 5,763,602; Martin et al, Methods Enzymol., 186: 635-645 (1990); Yarmush et al, Crit. Rev. Therapeutic Drug Carrier Syst., 10: 197-252 (1993); Pease et al, U.S. Pat. No. 5,709,994; Ullman et al, U.S. Pat. No. 5,340,716; Ullman et al, U.S. Pat. No. 6,251,581; McCapra, U.S. Pat. No. 5,516,636; Thetford, European patent publ. 0484027; Sessler et al, SPIE, 1426: 318-329 (1991); Magda et al, U.S. Pat. No. 5,565,552; Roelant, U.S. Pat. No. 6,001,673; and the like.

As with sensitizers, in certain embodiments, a photosensitizer may be associated with a solid phase support by being covalently or non-covalently attached to the surface of the support or incorporated into the body of the support. In general, the photosensitizer is associated with the support in an amount necessary to achieve the necessary amount of singlet oxygen. Generally, the amount of photosensitizer is determined empirically. In one preferred embodiment, a photosensitizer is incorporated into a latex particle to form photosensitizer beads, e.g. as disclosed by Pease et al., U.S. Pat. No. 5,709,994; Pollner, U.S. Pat. No. 6,346,384; and Pease et al, PCT publication WO 01/84157. Use of such photosensitizer beads is illustrated in FIG. 2C. As described in FIG. 1B for heteroduplex detection, complexes (230) are formed after combining reagents (122) with a sample. In this case, instead of attaching a photosensitizer directly to a binding compound, such as an antibody, a cleaving probe comprises two components: antibody (232) derivatized with a capture moiety, such as biotin (indicated in FIG. 2C as “bio”) and photosensitizer bead (238) whose surface is derivatized with an agent (234) that specifically binds with the capture moiety, such as avidin or streptavidin. Complexes (230) are then captured (236) by photosensitizer beads by way of the capure moiety. Photosensitizer beads may be used in either homogeneous or heterogeneous assay formats.

In another exemplary embodiment, the photosensitizer rose bengal is covalently attached to 0.5 micron latex beads by means of chloromethyl groups on the latex to provide an ester linking group, as described in J. Amer. Chem. Soc., 97: 3741 (1975).

### Assay Conditions

The following general discussion of methods and specific conditions and materials are by way of illustration and not limitation. One of ordinary skill in the art will understand how the methods described herein can be adapted to other applications, particularly with using different samples, cell types and target complexes.

In conducting the methods of the invention, a combination of the assay components is made, including the sample being tested, the binding compounds, and optionally the cleaving probe. Generally, assay components may be combined in any order. In certain applications, however, the order of addition may be relevant. For example, one may wish to monitor competitive binding, such as in a quantitative assay. Or one may wish to monitor the stability of an assembled complex. In such applications, reactions may be assembled in stages, and may require incubations before the complete mixture has been assembled, or before the cleaving reaction is initiated.

The amounts of each reagent are usually determined empirically. The amount of sample used in an assay will be determined by the predicted number of target complexes present and the means of separation and detection used to monitor the signal of the assay. In general, the amounts of the binding compounds and the cleaving probe are provided in molar excess relative to the expected amount of the target molecules in the sample, generally at a molar excess of at least 1.5, more desirably about 10-fold excess, or more. In specific applications, the concentration used may be higher or lower, depending on the affinity of the binding agents and the expected number of target molecules present on a single cell. Where one is determining the effect of a chemical compound on formation of oligomeric cell surface complexes, the compound may be added to the cells prior to, simultaneously with, or after addition of the probes, depending on the effect being monitored.

The assay mixture is combined and incubated under conditions that provide for binding of the probes to the cell surface molecules, usually in an aqueous medium, generally at a physiological pH (comparable to the pH at which the cells are cultures), maintained by a buffer at a concentration in the range of about 10 to 200 mM. Conventional buffers may be used, as well as other conventional additives as necessary, such as salts, growth medium, stabilizers, etc. Physiological and constant temperatures are normally employed. Incubation temperatures normally range from about 4° to 70° C., usually from about 15° to 45° C., more usually 25°to 37°.

After assembly of the assay mixture and incubation to allow the probes to bind to cell surface molecules, the mixture is treated to activate the cleaving agent to cleave the tags from the binding compounds that are within the effective proximity of the cleaving agent, releasing the corresponding tag from the cell surface into solution. The nature of this treatment will depend on the mechanism of action of the cleaving agent. For example, where a photosensitizer is employed as the cleaving agent, activation of cleavage will comprise irradiation of the mixture at the wavelength of light appropriate to the particular sensitizer used.

Following cleavage, the sample is then analyzed to determine the identity of tags that have been released. Where an assay employing a plurality of binding compounds is employed, separation of the released tags will generally precede their detection. The methods for both separation and detection are determined in the process of designing the tags for the assay. A preferred mode of separation employs electrophoresis, in which the various tags are separated based on known differences in their electrophoretic mobilities.

As mentioned above, in some embodiments, if the assay reaction conditions may interfere with the separation technique employed, it may be necessary to remove, or exchange, the assay reaction buffer prior to cleavage and separation of the molecular tags. For example, assay conditions may include salt concentrations (e.g. required for specific binding) that degrade separation performance when molecular tags are separated on the basis of electrophoretic mobility. Thus, such high salt buffers may be removed, e.g. prior to cleavage of molecular tags, and replaced with another buffer suitable for electrophoretic separation through filtration, aspiration, dilution, or other means.

### Separation of Released Molecular Tags

As mentioned above, molecular tags are designed for separation by a separation technique that can distinguish molecular tags based on one or more physical, chemical, and/or optical characteristics (referred to herein as “separation characteristics”). As also mentioned above, separation techniques that may be used with the various embodiments of the invention include normal phase or reverse phase HPLC, ion exchange HPLC, capillary electrochromatography, mass spectroscopy, gas phase chromatography, and the like. Preferably, the separation technique selected is capable of providing quantitative information as well as qualitative information about the presence or absence of molecular tags (and therefore, corresponding analytes). In one aspect, a liquid phase separation technique is employed so that a solution, e.g. buffer solution, reaction solvent, or the like, containing a mixture of molecular tags is processed to bring about separation of individual kinds of molecular tags. Usually, such separation is accompanied by the differential movement of molecular tags from such a starting mixture along a path until discernable peaks or bands form that correspond to regions of increased concentration of the respective molecular tags. Such a path may be defined by a fluid flow, electric field, magnetic field, or the like. The selection of a particular separation technique depends on several factors including the expense and convenience of using the technique, the resolving power of the technique given the chemical nature of the molecular tags, the number of molecular tags to be separated, the type of detection mode employed, and the like. Preferably, molecular tags are electrophoretically separated to form an electropherogram in which the separated molecular tags are represented by distinct peaks.

Methods for electrophoresis of are well known and there is abundant guidance for one of ordinary skill in the art to make design choices for forming and separating particular pluralities of molecular tags. The following are exemplary references on electrophoresis: Krylov et al, Anal. Chem., 72: 111R-128R (2000); P. D. Grossman and J. C. Colburn, Capillary Electrophoresis: Theory and Practice, Academic Press, Inc., NY (1992); U.S. Pat. Nos. 5,374,527; 5,624,800; 5,552,028; ABI PRISM 377 DNA Sequencer User's Manual, Rev. A, January 1995, Chapter 2 (Applied Biosystems, Foster City, Calif.); and the like. In one aspect, molecular tags are separated by capillary electrophoresis. Design choices within the purview of those of ordinary skill include but are not limited to selection of instrumentation from several commercially available models, selection of operating conditions including separation media type and concentration, pH, desired separation time, temperature, voltage, capillary type and dimensions, detection mode, the number of molecular tags to be separated, and the like.

In one aspect of the invention, during or after electrophoretic separation, the molecular tags are detected or identified by recording fluorescence signals and migration times (or migration distances) of the separated compounds, or by constructing a chart of relative fluorescent and order of migration of the molecular tags (e.g., as an electropherogram). Preferably, the presence, absence, and/or amounts of molecular tags are measured by using one or more standards as disclosed by Williams et al, U.S. patent publication 2003/0170734A1, which is incorporated herein by reference. During or after separation, fluorescent molecular tags can be illuminated by standard means, e.g. a high intensity mercury vapor lamp, a laser, or the like. Typically, the molecular tags are illuminated by laser light generated by a He—Ne gas laser or a solid-state diode laser. The fluorescence signals can then be detected by a light-sensitive detector, e.g., a photomultiplier tube, a charged-coupled device, or the like. Exemplary electrophoresis detection systems are described elsewhere, e.g., U.S. Pat. Nos. 5,543,026; 5,274,240; 4,879,012; 5,091,652; 6,142,162; or the like. In another aspect, molecular tags may be detected electrochemically detected, e.g. as described in U.S. Pat. No. 6,045,676.

Electrophoretic separation involves the migration and separation of molecules in an electric field based on differences in mobility. Various forms of electrophoretic separation include, by way of example and not limitation, free zone electrophoresis, gel electrophoresis, isoelectric focusing, isotachophoresis, capillary electrochromatography, and micellar electrokinetic chromatography. Capillary electrophoresis involves electroseparation, preferably by electrokinetic flow, including electrophoretic, dielectrophoretic and/or electroosmotic flow, conducted in a tube or channel of from about 1 I to about 200 micrometers, usually, from about 10 to about 100 micrometers cross-sectional dimensions. The capillary may be a long independent capillary tube or a channel in a wafer or film comprised of silicon, quartz, glass or plastic.

In capillary electroseparation, an aliquot of the reaction mixture containing the molecular tags is subjected to electroseparation by introducing the aliquot into an electroseparation channel that may be part of, or linked to, a capillary device in which the amplification and other reactions are performed. An electric potential is then applied to the electrically conductive medium contained within the channel to effectuate migration of the components within the combination. Generally, the electric potential applied is sufficient to achieve electroseparation of the desired components according to practices well known in the art. One skilled in the art will be capable of determining the suitable electric potentials for a given set of reagents used in the present invention and/or the nature of the cleaved labels, the nature of the reaction medium and so forth. The parameters for the electroseparation including those for the medium and the electric potential are usually optimized to achieve maximum separation of the desired components. This may be achieved empirically and is well within the purview of the skilled artisan. Detection may be by any of the known methods associated with the analysis of capillary electrophoresis columns including the methods shown in U.S. Pat. No. 5,560,811 (column 11, lines 19-30), U.S. Pat. Nos. 4,675,300, 4,274,240 and 5,324,401, the relevant disclosures of which are incorporated herein by reference. Those skilled in the electrophoresis arts will recognize a wide range of electric potentials or field strengths may be used, for example, fields of 10 to 1000 V/cm are used with about 200 to about 600 V/cm being more typical. The upper voltage limit for commercial systems is about 30 kV, with a capillary length of about 40 to about 60 cm, giving a maximum field of about 600 V/cm. For DNA, typically the capillary is coated to reduce electroosmotic flow, and the injection end of the capillary is maintained at a negative potential.

For ease of detection, the entire apparatus may be fabricated from a plastic material that is optically transparent, which generally allows light of wavelengths ranging from about 180 to about 1500 nm, usually about 220 to about 800 nm, more usually about 450 to about 700 nm, to have low transmission losses. Suitable materials include fused silica, plastics, quartz, glass, and so forth.

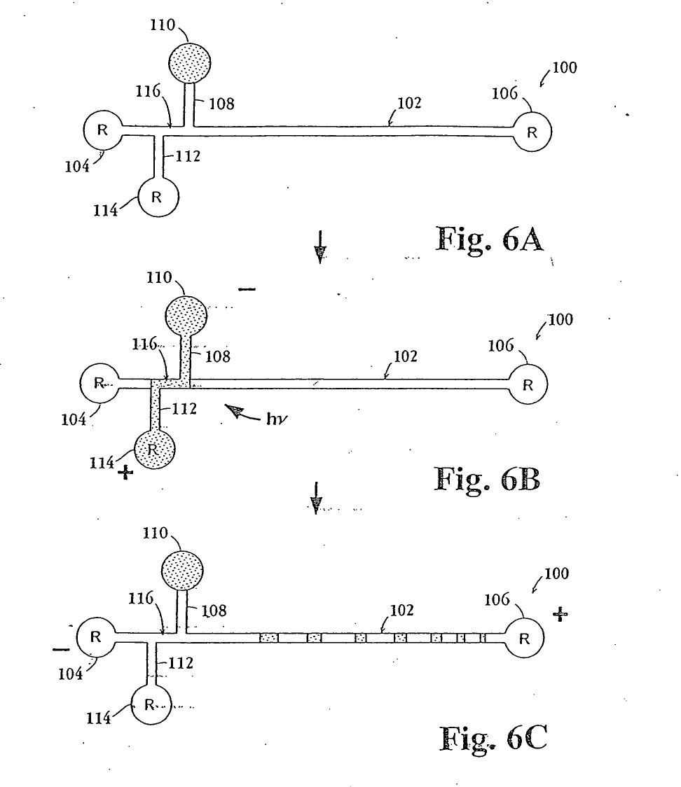

In one aspect of the invention, molecular tags are separated by electrophoresis in a microfluidics device, as illustrated diagrammatically in FIGS. 6A-6C. Microfluidics devices are described, for example, in U.S. Pat. Nos. 5,750,015; 5,900,130; 6,007,690; and WO 98/45693; WO 99/19717 and WO 99/15876. Conveniently, an aliquot, generally not more than about 5 μl, is transferred to the sample reservoir of a microfluidics device, either directly through electrophoretic or pneumatic injection into an integrated system or by syringe, capillary or the like. The conditions under which the separation is performed are conventional and will vary with the nature of the products.

By way of illustration, FIGS. 6A-6C show a microchannel network 100 in a microfluidics device of the type detailed in the application noted above, for sample loading and electrophoretic separation of a sample of probes and tags produced in the assay above. Briefly, the network includes a main separation channel 102 terminating at upstream and downstream reservoirs 104, 106, respectively. The main channel is intersected at offset axial positions by a side channel 108 that terminates at a reservoir 110, and a side channel 112 that terminates at a reservoir 114. The offset between the two-side channels forms a sample loading zone 116 within the main channel.

In operation, an assay mixture is placed in sample reservoir 110, illustrated in FIG. 6A. As noted, the assay mixture contains one or more target cells with surface-bound cleaving agent, one or more protein probes, and optionally, molecular tag standard. The assay reaction, involving initial probe binding to target cell(s), followed by cleavage of probe linkers in probe-bound cells, may be carried out in sample reservoir 110, or alternatively, the assay reactions can be carried out in another reaction vessel, with the reacted sample components the added to the sample reservoir.

To load released molecular tags into the sample-loading zone, an electric field is applied across reservoirs 110, 114, in the direction indicated in FIG. 6B, wherein negatively charged released molecular tags are drawn from reservoir 110 into loading zone 116, while uncharged or positively charged sample components remain in the sample reservoir. The released tags in the loading zone can now be separated by conventional capillary electrophoresis, by applying an electric filed across reservoirs 104, 106, in the direction indicated in FIG. 6C.

From the resulting electrophoretic pattern, the molecular tags, and corresponding analytes, can be identified. This is typically done by placing a fluorescence detector near the downstream end of the separation channel, and constructing a electropherogram of the separated molecular tags, first to determine the separation characteristic (in this case, electrophoretic mobility) as above, and secondly, to measure signal intensity, e.g., peak height or peak area, as a measure of the relative amount of tag associated with each probe. Methods for detecting and quantifying levels of a detectable probe are well known. In one preferred method, the molecular tags are fluorescent labeled. A standard fluorescence-emission source is directed against a detection zone in a downstream portion of the separation medium, and fluorescence emission of the zone is measured by a standard light detector. The signal height or area recorded provides a measure of product and substrate concentration in the sample. With the above detection information, it is now possible to assign each detected molecular tag to a particular probe in the probe set, and to compare the relative levels of each detectable probe, as a measure of its relatively substrate conversion or ligand binding.

In one aspect of the invention, pluralities of molecular tags are designed for separation by chromatography based on one or more physical characteristics that include but are not limited to molecular weight, shape, solubility, pKa, hydrophobicity, charge, polarity, or the like. A chromatographic separation technique is selected based on parameters such as column type, solid phase, mobile phase, and the like, followed by selection of a plurality of molecular tags that may be separated to form distinct peaks or bands in a single operation. Several factors determine which HPLC technique is selected for use in the invention, including the number of molecular tags to be detected (i.e. the size of the plurality), the estimated quantities of each molecular tag that will be generated in the assays, the availability and ease of synthesizing molecular tags that are candidates for a set to be used in multiplexed assays, the detection modality employed, and the availability, robustness, cost, and ease of operation of HPLC instrumentation, columns, and solvents. Generally, columns and techniques are favored that are suitable for analyzing limited amounts of sample and that provide the highest resolution separations. Guidance for making such selections can be found in the literature, e.g. Snyder et al, Practical HPLC Method Development, (John Wiley & Sons, New York, 1988); Millner, “High Resolution Chromatography: A Practical Approach”, Oxford University Press, New York (1999), Chi-San Wu, “Column Handbook for Size Exclusion Chromatography”, Academic Press, San Diego (1999), and Oliver, “HPLC of Macromolecules: A Practical Approach, Oxford University Press”, Oxford, England (1989). In particular, procedures are available for systematic development and optimization of chromatographic separations given conditions, such as column type, solid phase, and the like, e.g. Haber et al, J. Chromatogr. Sci., 38: 386-392 (2000); Outinen et al, Eur. J. Pharm. Sci., 6: 197-205 (1998); Lewis et al, J. Chromatogr., 592: 183-195 and 197-208 (1992); and the like.

In one aspect, initial selections of molecular tag candidates are governed by the physiochemical properties of molecules typically separated by the selected column and stationary phase. The initial selections are then improved empirically by following conventional optimization procedure, as described in the above reference, and by substituting more suitable candidate molecular tags for the separation objectives of a particular embodiment. In one aspect, separation objectives of the invention include (i) separation of the molecular tags of a plurality into distinguishable peaks or bands in a separation time of less than 60 minutes, and more preferably in less than 40 minutes, and still more preferably in a range of between 10 to 40 minutes, (ii) the formation of peaks or bands such that any pair has a resolution of at least 1.0, more preferably at least 1.25, and still more preferably, at least 1.50, (iii) column pressure during separation of less than 150 bar, (iv) separation temperature in the range of from 25° C. to 90° C., preferably in the range of from 35° C. to 80° C., and (v) the plurality of distinguishable peaks is in the range of from 5 to 30 and all of the peaks in the same chromatogram. As used herein, “resolution” in reference to two peaks or bands is the distance between the two peak or band centers divided by the average base width of the peaks, e.g. Snyder et al (cited above). A variety of commercially available systems are well-suited for high throughput chromatographic analysis of molecular tags. Those skilled in the art can determine appropriate equipment, such as automated sample preparation systems and autoinjection systems, useful for automating HPLC analysis of molecular tags. Automated methods can be used for high-throughput analysis of molecular tags, for example, when a large number of samples are being processes or for multiplexed application of the methods of the invention for detecting target analytes. An exemplary HPLC instrumentation system suitable for use with the present invention is the Agilent 1100 Series HPLC system (Agilent Technologies, Palo Alto, Calif.).

### Synthesis of Assay Reagents

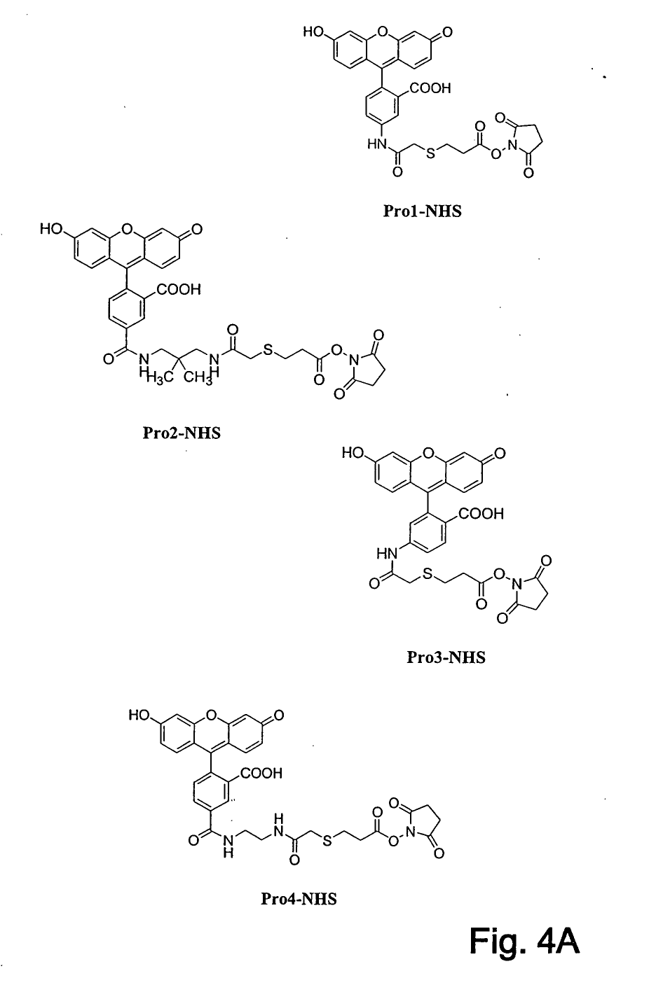

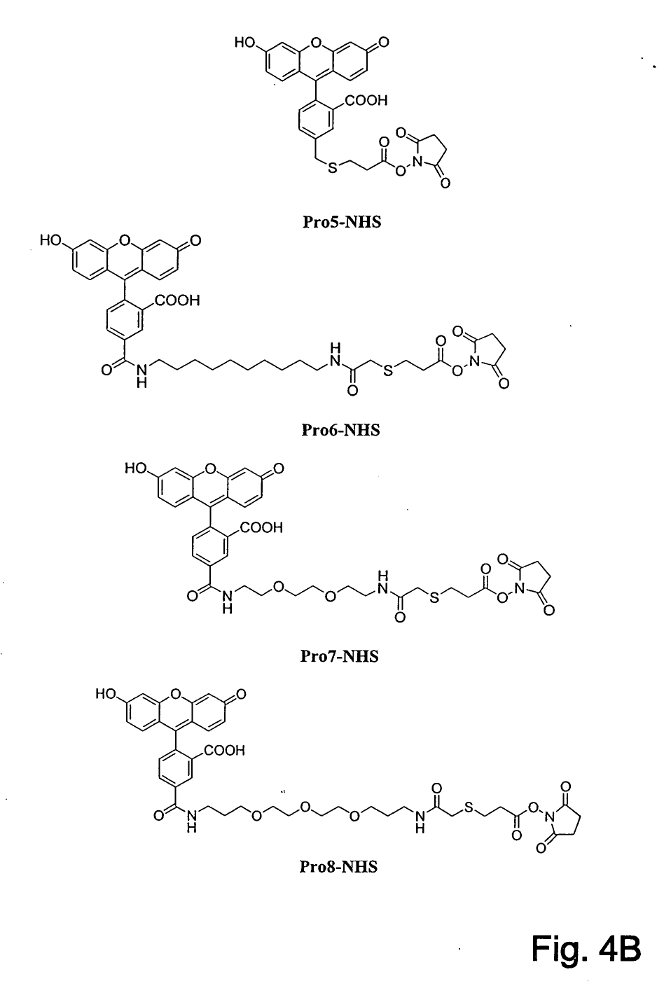

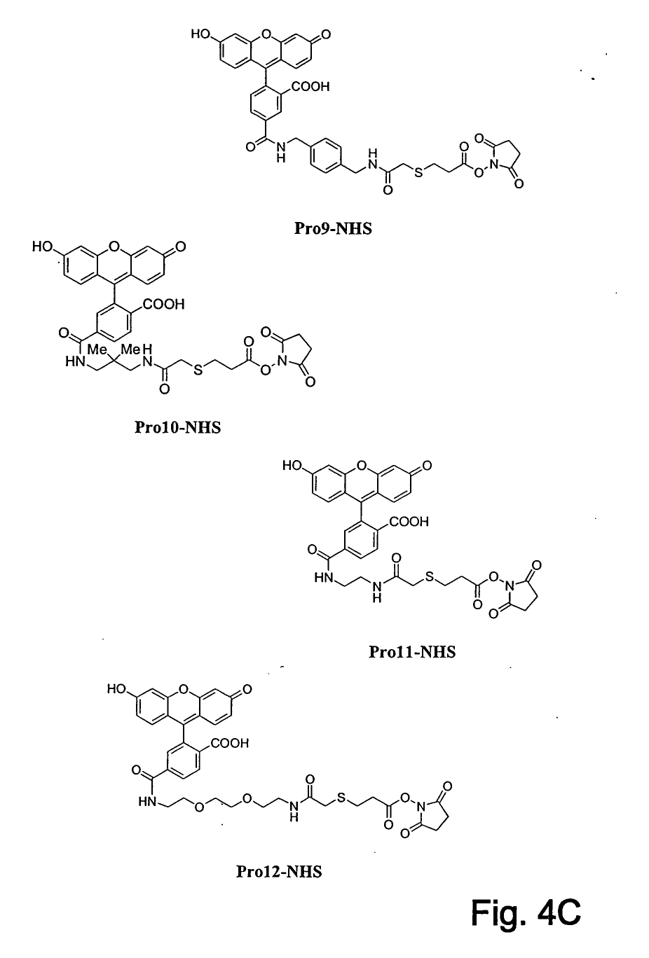

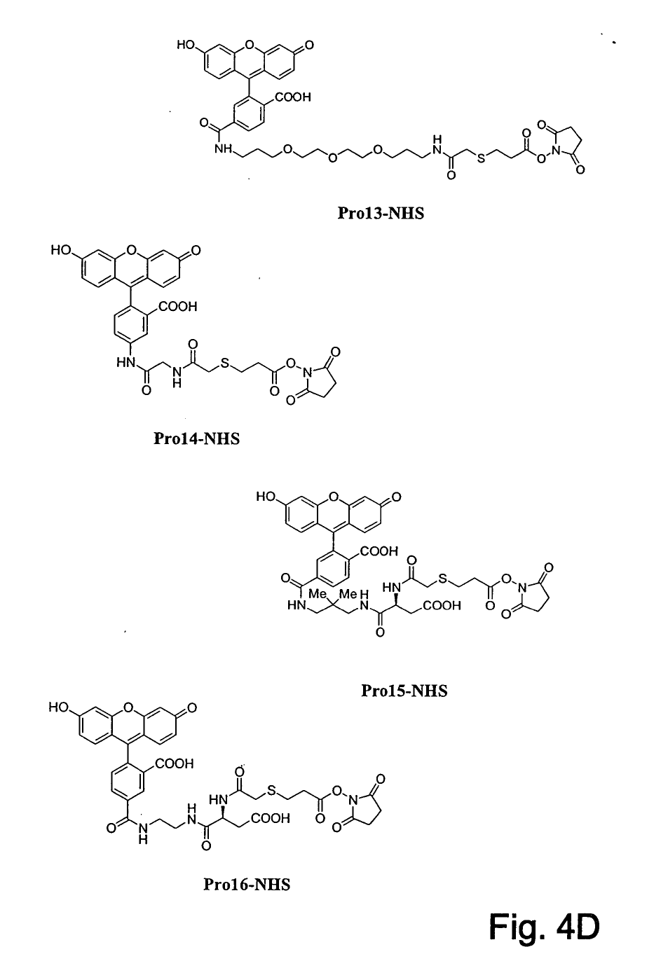

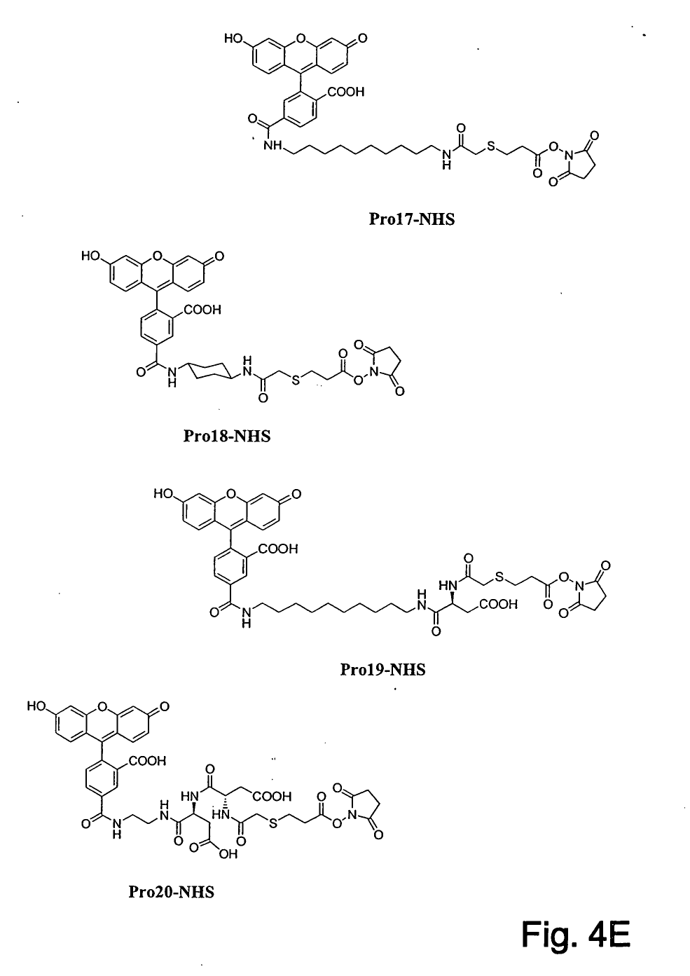

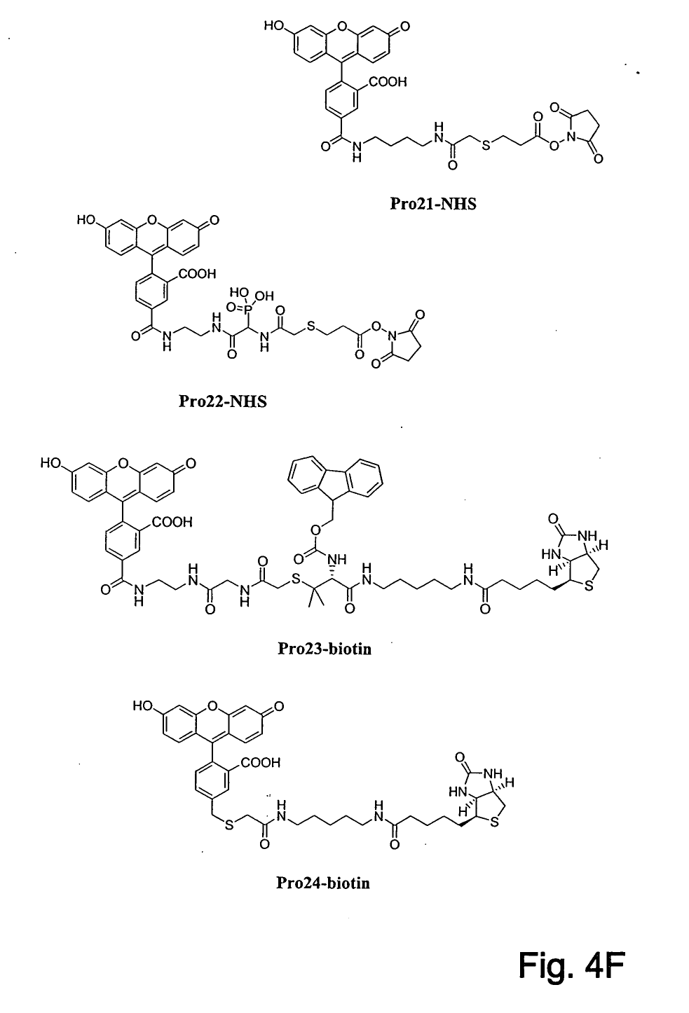

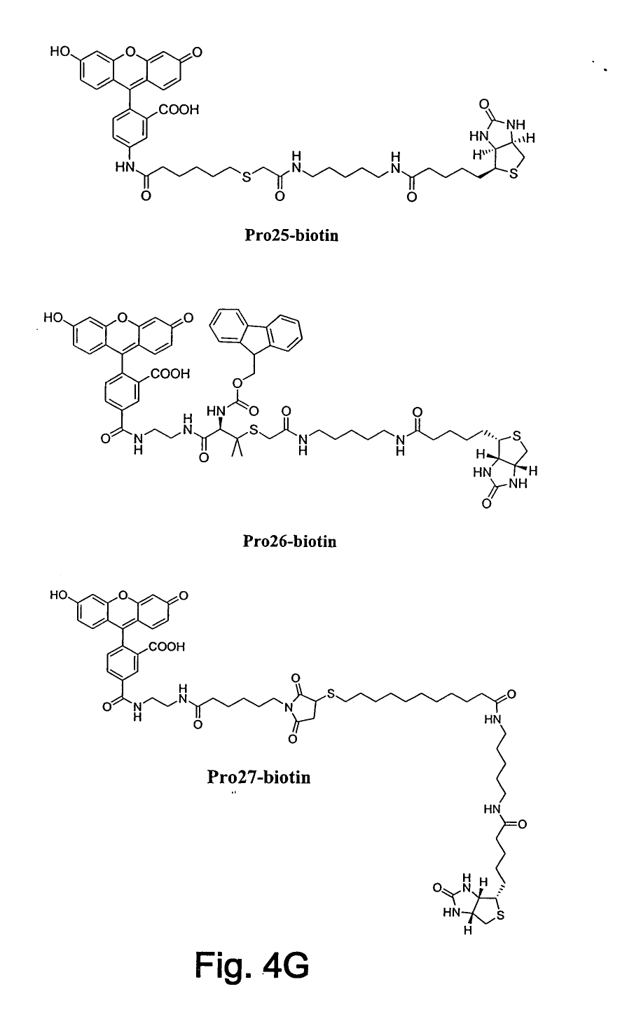

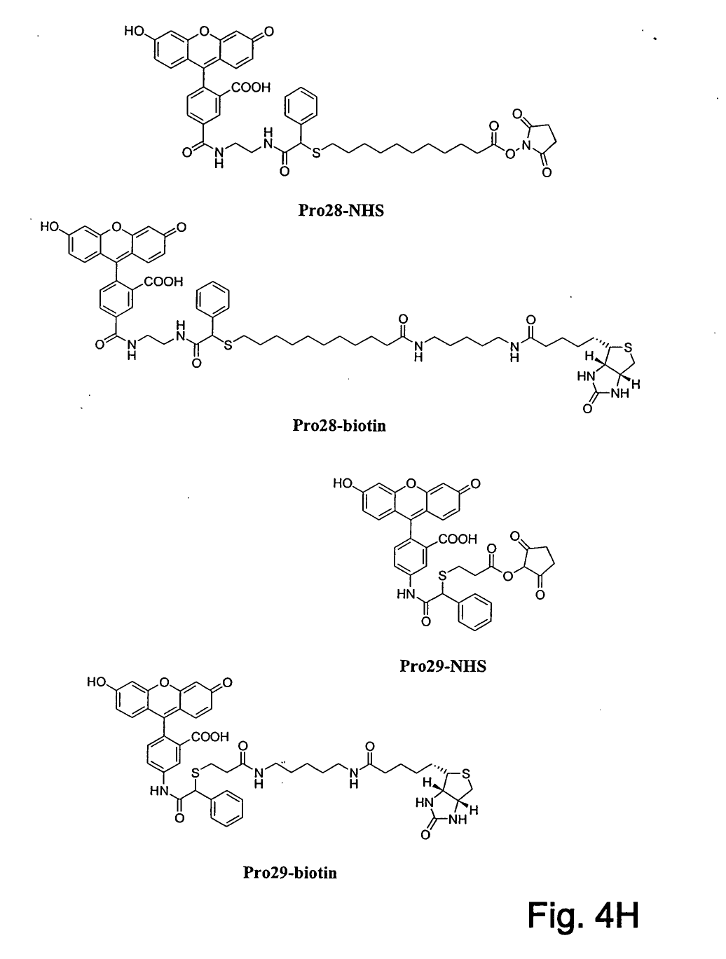

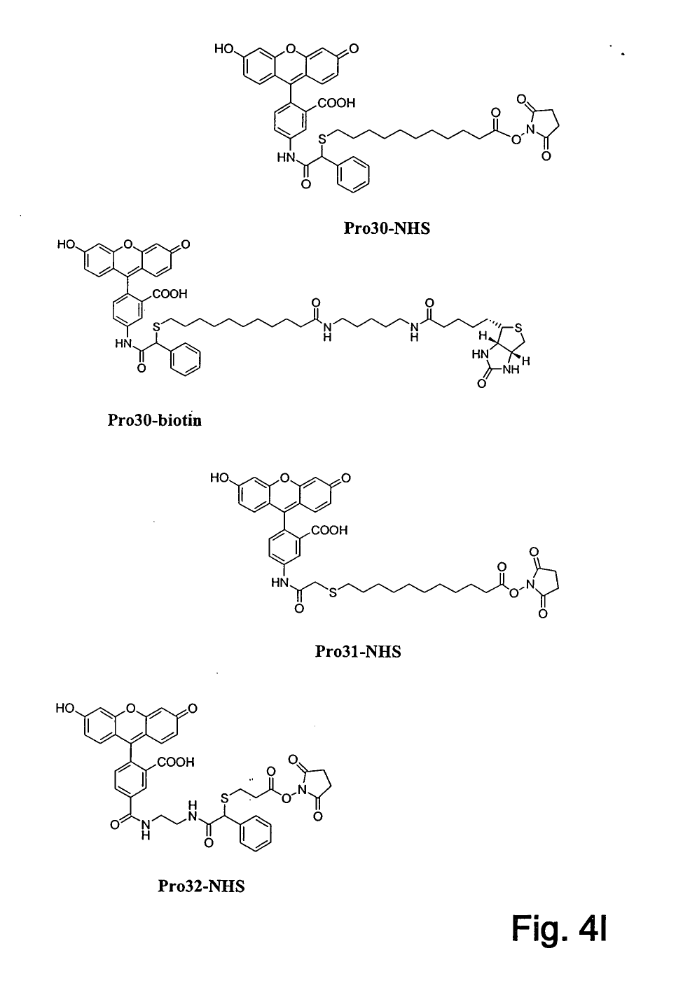

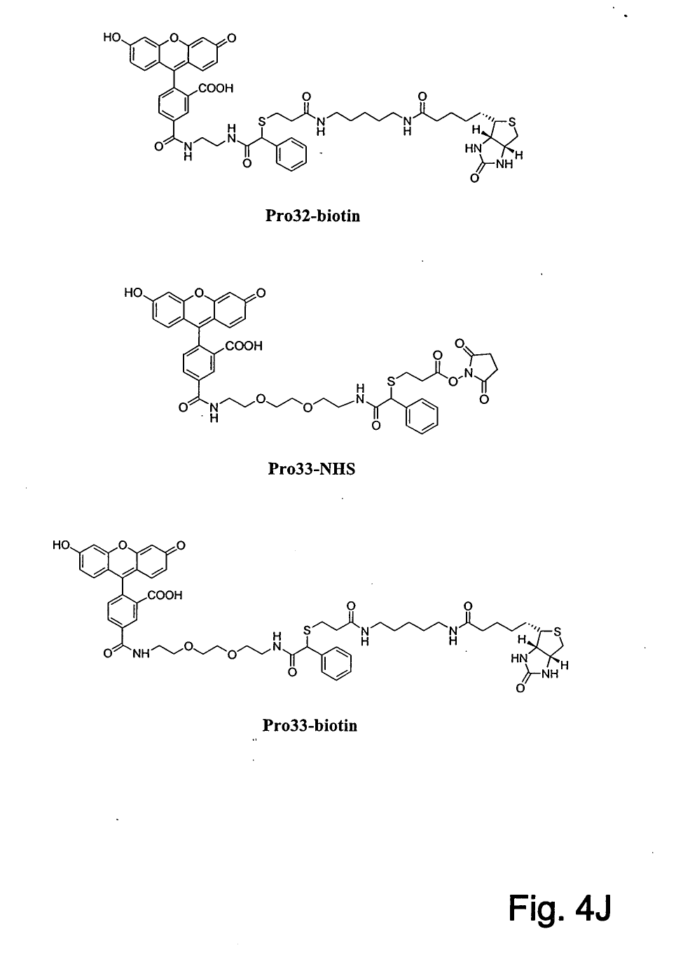

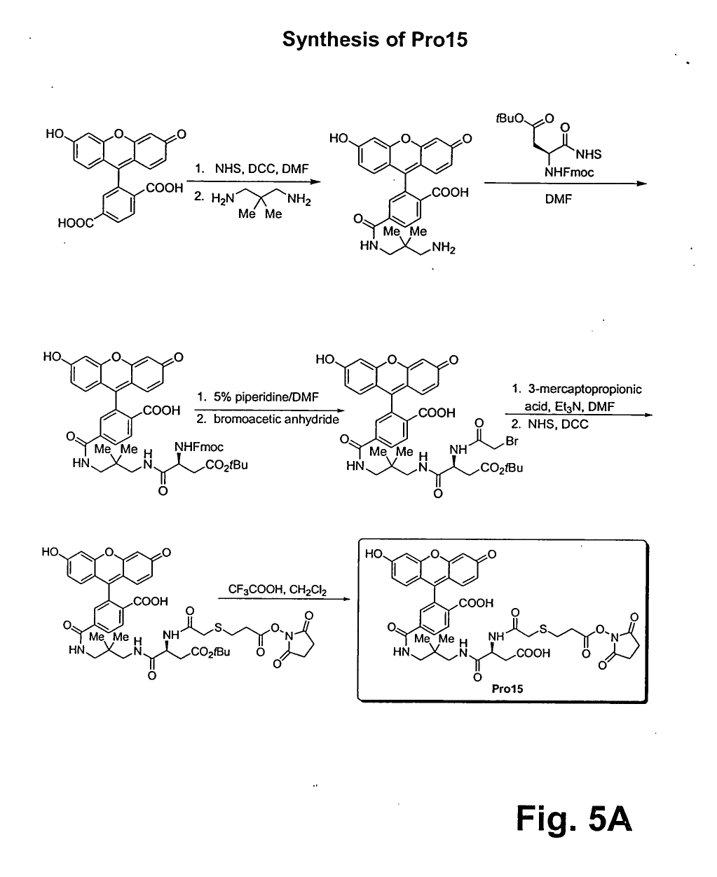

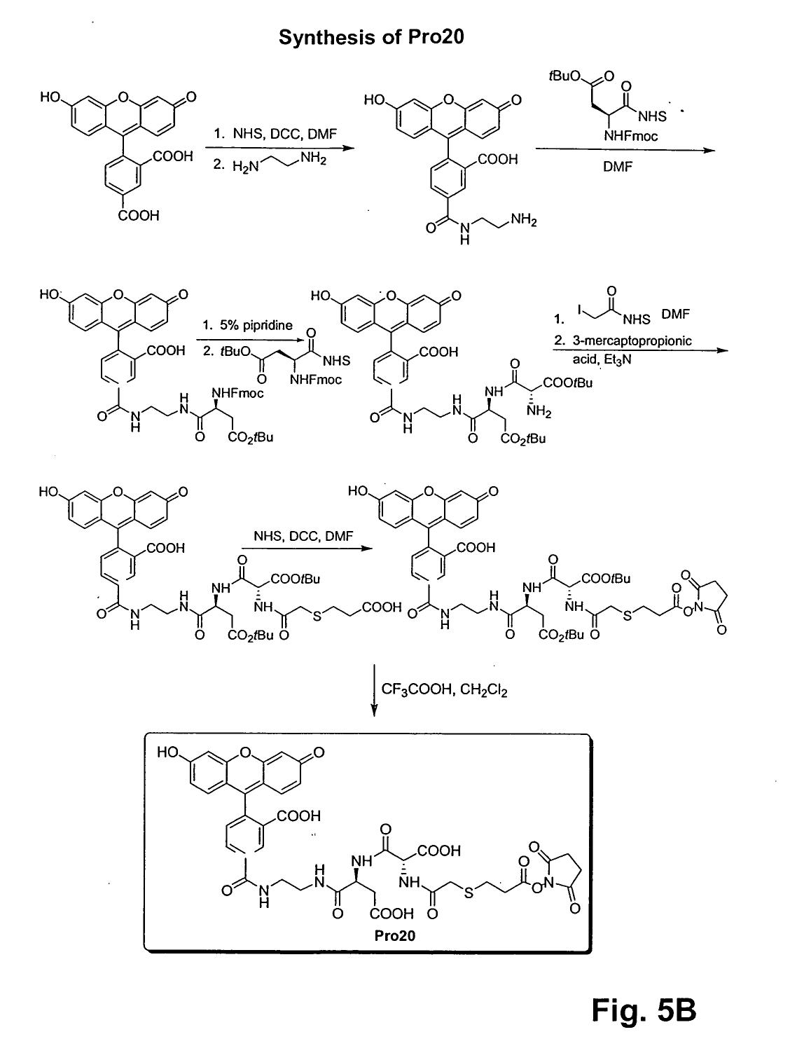

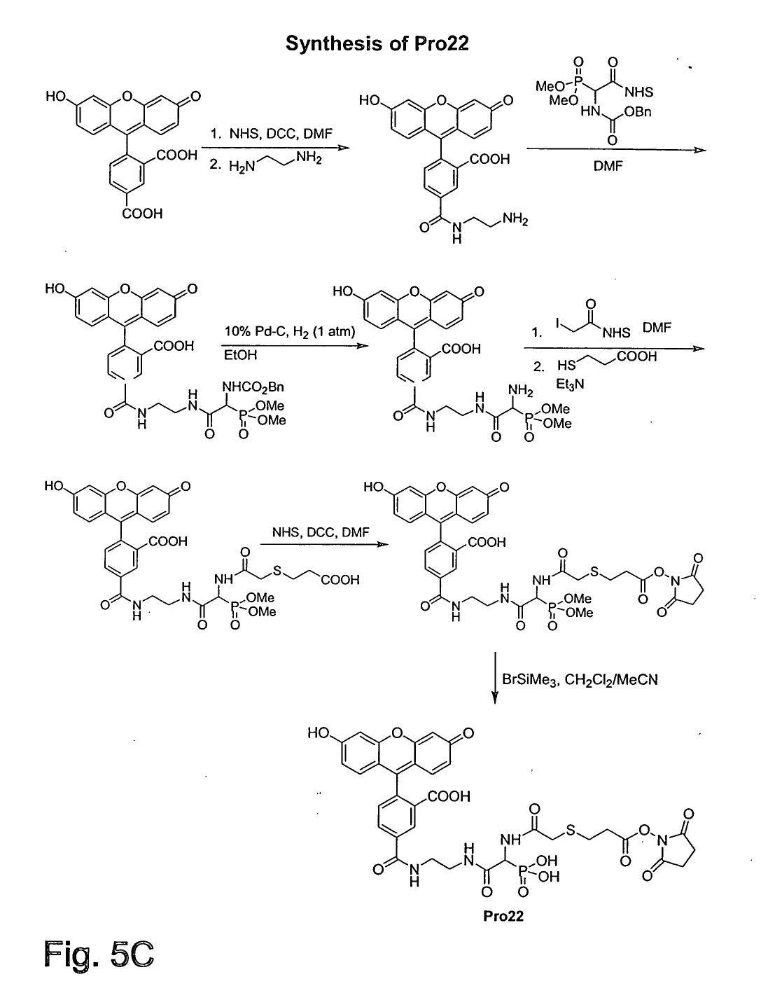

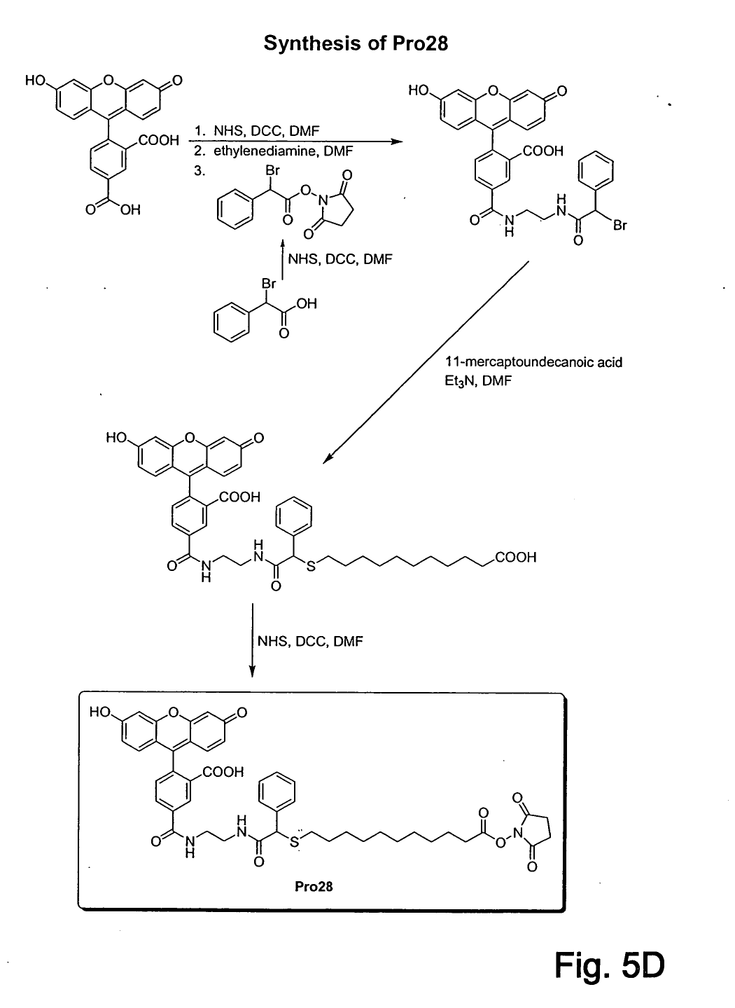

Binding compounds for use of the invention are synthesized as disclosed in the following references, which are incorporated herein by reference: International patent publications WO 00/66607; WO 01/83502; WO 02/95356; WO 03/06947; and U.S. Pat. Nos. 6,322,980 and 6,514,700. Exemplary reagents for synthesis of binding compounds are shown in FIGS. 4A-J. Exemplary synthesis protocols are illustrated in FIGS. 5A-7D.

### EXAMPLES

### Sources of Materials Used in Examples

Antibodies specific for Her receptors, adaptor molecules, and normalization standards are obtained from commercial vendors, including Labvision, Cell Signaling Technology, and BD Biosciences. All cell lines were purchased from ATCC. All human snap-frozen tissue samples were purchased from either William Bainbridge Genome Foundation (Seattle, Wash.) or Bio Research Support (Boca Raton, Fla.) and were approved by Institutional Research Board (IRB) at the supplier.

The molecular tag-antibody conjugates used below are formed by reacting NHS esters of the molecular tag with a free amine on the indicated antibody using conventional procedures. Molecular tags, identified below by their “Pro_N” designations, are disclosed above and in the following references: Singh et al, U.S. patent publications, 2003/017915 and 2002/0013126, which are incorporated by reference. Briefly, binding compounds below are molecular tag-monoclonal antibody conjugates formed by reacting an NHS ester of a molecular tag with free amines of the antibodies in a conventional reaction.

### Example 1

### PI3K/Her-3 Receptor Activation Complex

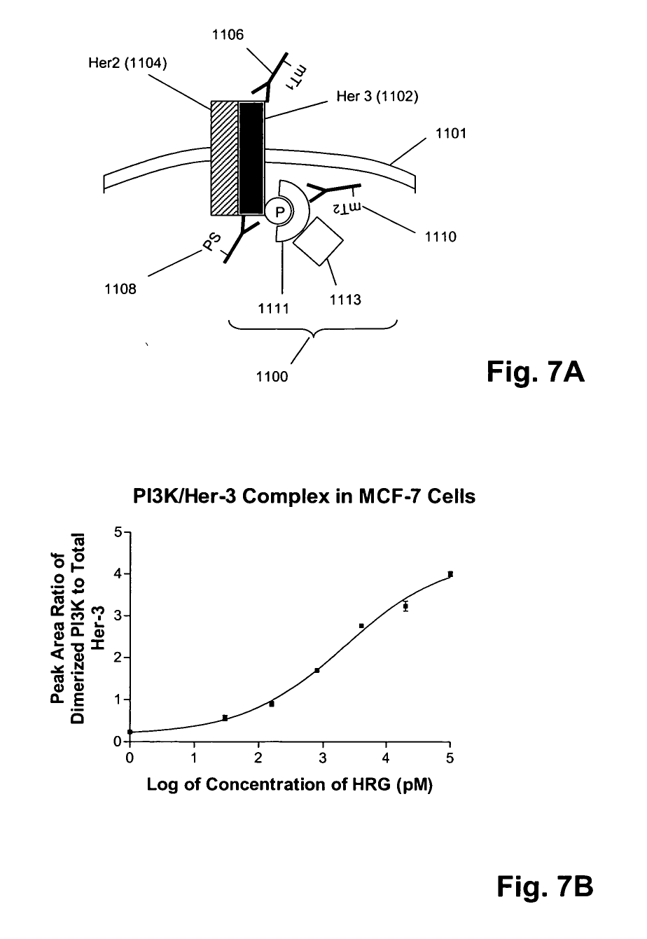

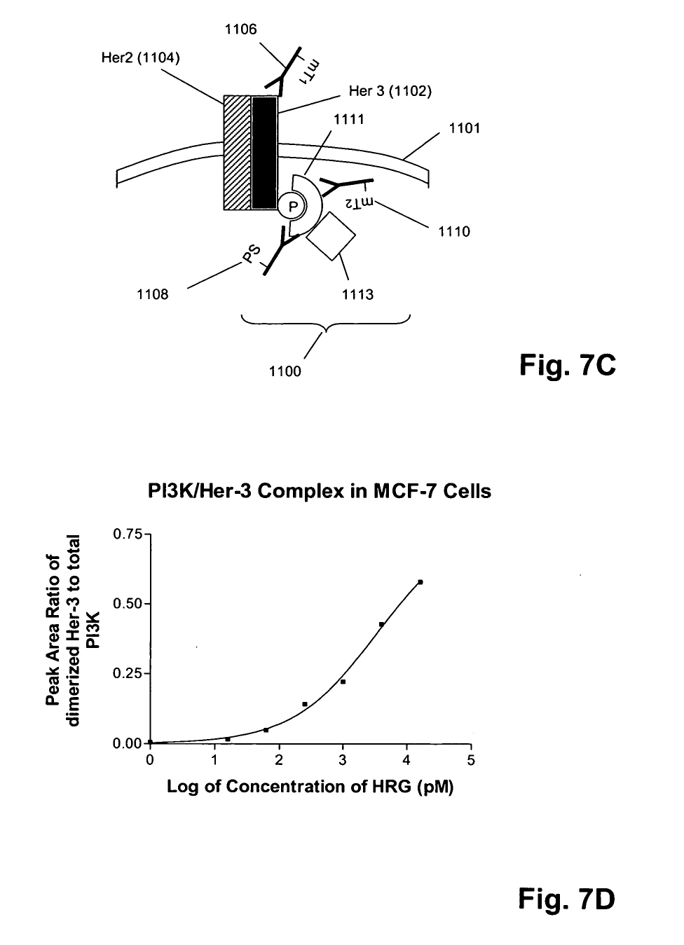

In this example, assays were designed as shown in FIGS. 7A and 7C to measure a receptor complex comprising Her2, Her3, and PI3K in breast cancer cell line, MCF-7. Binding compound (1106) having a first molecular tag (“mT₁” in the figure and “eTag1” below) is specific for the extracellular domain of Her3 receptor (1102), binding compound (1110) having a second molecular tag (“mT2” in the figure and “eTag2” below) is specific for the p185 component (1111) of PI3K protein (1100), and cleaving probe (1108) having a photosensitizer attached (is specific for the intracellular domain of Her3 receptor (1102). The two assay designs are similar, except that in the design of FIG. 7A the cleaving probe is specific for the Her3 receptor, and in the design of FIG. 7C, the cleaving probe is specific for the p85 component (1111) of PI3 kinase. The assays were carried out as follows. Sample Preparation:

1. Serum-starve breast cancer cell line culture overnight before use.

2. Stimulate cell lines with HRG in culture media for 10 minutes at 37° C. Exemplary doses of HRG are 0, 0.032, 0.16, 0.8, 4, 20, 100 nM for MCF-7 cells.

3. Aspirate culture media, transfer onto ice, and add lysis buffer (described above to lyse cells in situ.

4. Scrape and transfer lysate to microfuge tube. Incubate on ice for 30 min. Microfuge at 14,000 rpm, 4° C., for 10 min.

5. Collect supernatants as lysates and aliquot for storage at −80° C. until use.

Lysis Buffer (Made Fresh and Stored on Ice):

| Final | ul | | Stock |
|---|---|---|---|
| 1% Triton X-100 | 1000 | | 10% |
| 20 mM Tris-HCl (pH 7.5) | 200 | | 1 M |
| 100 mM NaCl | 200 | | 5 M |
| 50 mM NaF | 500 | | 1 M |
| 50 mM Na beta-glycerophosphate | 1000 | | 0.5 M |
| 1 mM Na₃VO₄ | 100 | | 0.1 M |
| 5 mM EDTA | 100 | | 0.5 M |
| 10 ug/ml pepstatin | 100 | | 1 mg/ml |
| 1 tablet (per 10 ml) Roche Complete protease | N/A | | N/A |
| inhibitor (#1836170) | | | |
| Water | 6500 | | N/A |
| | 10 | ml | Total |

Assay design: Receptor complex formation is quantified ratiometrically based on the schematics illustrated in each figure. That is, the readout of the assays are the peak ratios of molecular tags, eTag2/eTag1.

The total assay volume is 40 ul. The lysate volume is adjusted to 10 ul with lysis buffer. The antibodies are diluted in lysis buffer up to 20 ul. Typically ˜5000 to 500,000 cell-equivalent of lysates is used per reaction.

Procedure: Working concentrations of pre-mixed antibodies prior to adding into reaction: For Her-3/PI3K complex with cleaving probe at Her-3 (the design of FIG. 11A)

eTag1_anti-Her-3 at 10 nM (eTag1 was Pro14 in this assay)

eTag2_anti-PI3K at 10 nM (eTag2 was Pro1 in this assay)

Biotin_anti-Her-3 at 20 nM

Universal Standard US-1 at 700 nM

[The Universal Standard US-1 is BSA conjugated with biotin and molecular tag Pro8, which is used to normalize the amount of streptavidin-photosensitizer beads in an assay]. The molecular tags were attached directly to antibodies by reacting an NHS-ester of a molecular tag precursor (see FIGS. 4A-4J) with free amines on the antibodies using conventional techniques, e.g. Hermanson (cited above).

For Her-3/PI3K complex with cleaving probe at PI3K (the design of FIG. 7C):

eTag1_anti-PI3K at 10 nM (eTag1 was Pro1 in this assay)

eTag2_anti-Her-3 at 10 nM (eTag2 was Pro14 in this assay)

Biotin_anti-PI3K at 20 nM

Universal Standard US-1 at 700 nM

1. To assay 96-well filter plate (Millipore MAGVN2250), add 20 ul antibody mix to 10 ul lysate and incubate for 1 hour at 4° C.

2. Add 10 ul streptavidin-derivatized cleaving probe (final 4 ug/well) to assay well and incubate for 40 min.

3. Add 200 ul wash buffer and apply vacuum to empty.

4. Add 30 ul illumination buffer and illuminate.

5. Transfer 10 ul of each reaction to CE assay plate for analysis.

Data Analysis:

1. Normalize relative fluorescence units (RFU) signal of each molecular tag against that of internal Universal Standard US-1.

2. Subtract RFU of “no lysate” background control from corresponding normalized eTag reporter signals.

3. Report receptor complex formation as the ratiometric of normalized eTag2/eTag1 signal (shown in FIGS. 7B and 7D).

### Example 2

### Shc/Her-3 Receptor-Adaptor Interaction

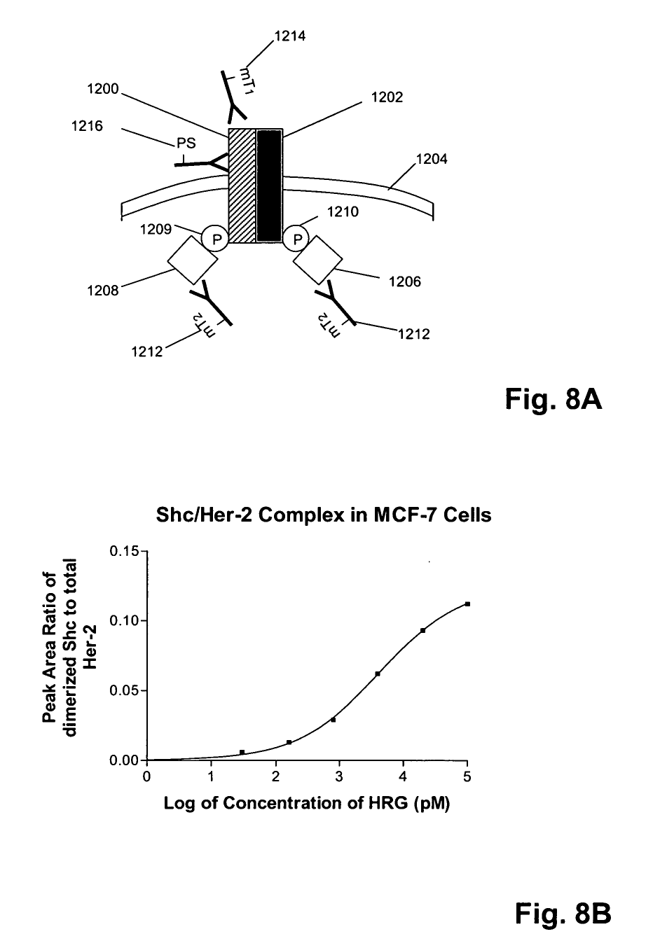

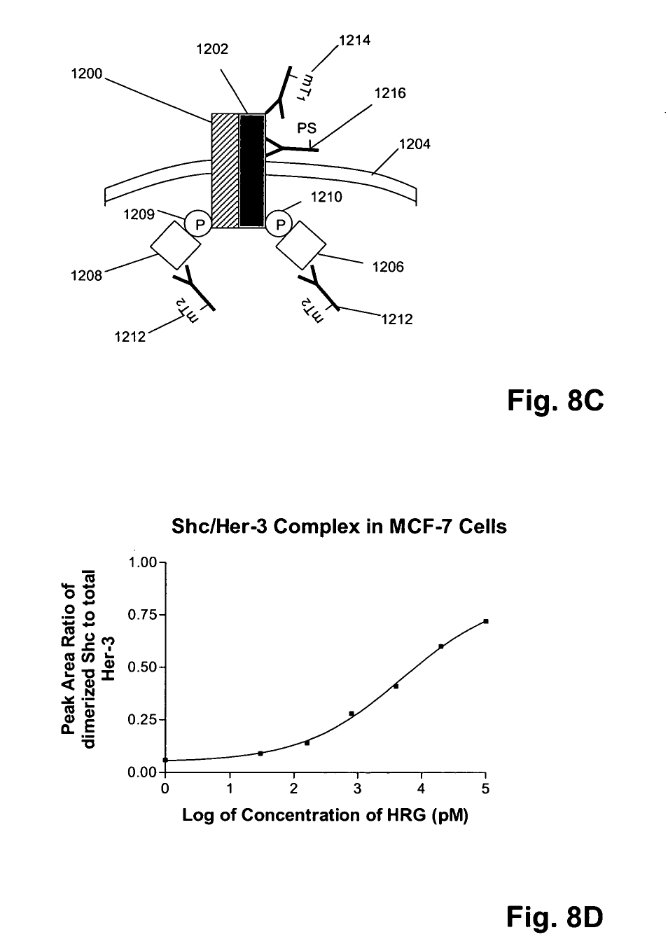

In this example, an assays were designed as shown in FIGS. 8A and 8C. In FIG. 8A, Her2 receptor (1200) and Her3 receptor (1202) form a dimer in cell surface membrane (1204) and each receptor is represented as having phosphorylated sites (1209 and 1210). Shc proteins (1206 and 1208) bind to phosphylation sites (1210) and (1209), respectively. A first binding compound (1214) and cleaving probe (1216) are specific for different antigenic determinants of the extracellular domain of Her2 receptor (1200). A second binding compound (1212) is specific for Shc proteins (1206 and 1208). The assay designs of FIGS. 8A and 8C are similar, except that in the design of FIG. 8A the cleaving probe is specific for the Her2 receptor, and in the design of FIG. 8C, the cleaving probe is specific for the Her3 receptor. Thus, in the former case, total Her2 receptor is measured, whereas in the latter case total Her3 receptor is measured. The assays were carried out as follows. Sample preparation was carried out as above (Example 1).

Assay design: Receptor complex formation is quantified ratiometrically based on the schematics illustrated in each figure. That is, in FIGS. 8B and 8D the readout of the assays are the peak ratios of mT₂/mT₁ as a function of HRG concentration.

The total assay volume is 40 ul. The lysate volume is adjusted to 10 ul with lysis buffer. The antibodies are diluted in lysis buffer up to 20 ul. Typically about 5000 to 500,000 cell-equivalent of lysates is used per reaction.

Procedure: Working concentrations of pre-mixed antibodies prior to adding into reaction:

For Her-3/Shc complex with cleaving probe at Her-3 (the design of FIG. 8B):

eTag1_anti-Her-3 at 10 nM (eTag1 was Pro14 in this assay)

eTag2_anti-Shc at 10 nM (eTag2 was Pro12 in this assay)

eTag3_anti-phospho-Tyr at 10 nM (eTag3 was Pro2 in this assay)

Biotin_anti-Her-3 at 20 nM

Universal Standard US-1 at 700 nM

For Her-2/Shc complex with cleaving probe at Her-2 (the design of 8A):

eTag1_anti-Her-2 at 10 nM (eTag1 was Pro14 in this assay)

eTag2_anti-Shc at 10 nM (eTag2 was Pro12 in this assay)

eTag3_anti-phospho-Tyr at 10 nM (eTag3 was Pro2 in this assay)

Biotin_anti-Her-2 at 20 nM

Universal Standard US-1 at 700 nM

1. To assay 96-well filter plate (Millipore MAGVN2250), add 20 ul antibody mix to 10 ul lysate and incubate for 1 hour at 4° C.

2. Add 10 ul streptavidin-derivatized cleaving probe (final 4 ug/well) to assay well and incubate for 40 min.

3. Add 200 ul wash buffer and apply vacuum to empty.

4. Add 30 ul illumination buffer and illuminate.

5. Transfer 10 ul of each reaction to CE assay plate for analysis.

Data Analysis:

1. Normalize relative fluorescence units (RFU) signal of each molecular tag against that of internal Universal Standard US-1.

2. Subtract RFU of “no lysate” background control from corresponding normalized signals for molecular tags.

3. Report receptor complex formation as the ratiometric of normalized mT₂/mT₁ signals (shown in FIGS. 8B and 8D) and receptor phosphorylation (data not shown) as mT3/mT1 signals.

### Example 3

### Simultaneous Measurement of 14-3-3//BAD and Bcl-2//BAD Apoptotic Complexes

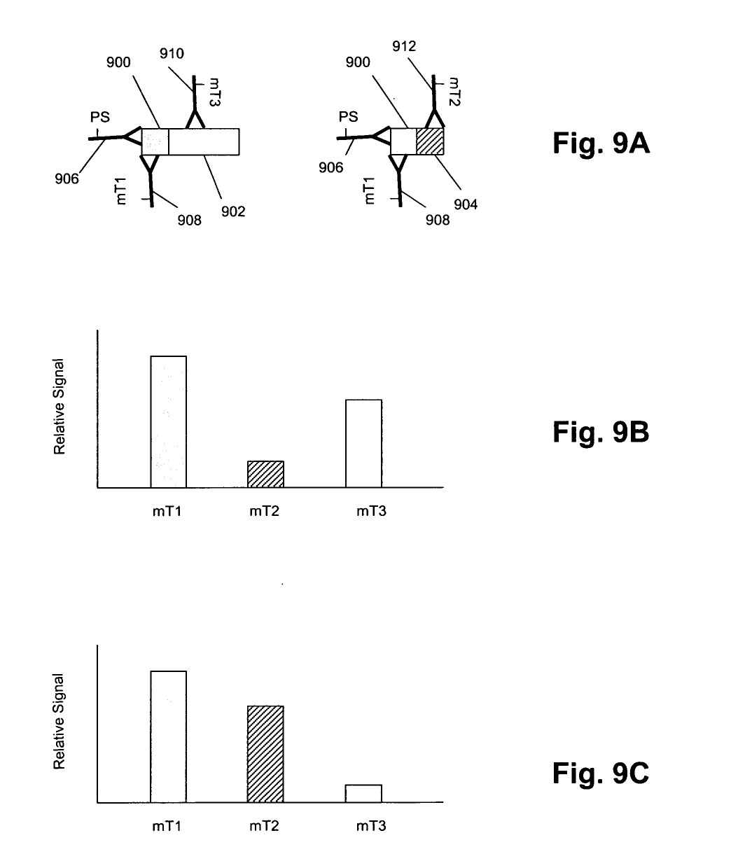

In this example, an assay, illustrated in FIGS. 9A-9C, is described for measuring relative amounts of two complexes that contain BAD protein: (i) 14-3-3 protein//BAD protein, and (ii) Bcl-2 protein//BAD protein. Cleaving probe (906) is specific for BAD protein (900) at a first antigenic determinant, binding compound (908) is specific for BAD protein (900) at a second antigenic determinant, binding compound (910) is specific for 14-3-3 protein (902), and binding compound (912) is specific for Bcl-2 protein (904). The cleaving probe and binding compounds are selected so that their binding sites do not interfere with one another and that site are not selected that are hidden when either complex forms. The assays are carried out as follows.

Sample Preparation:

1. Serum-starve breast cancer cell line culture (MCF-7) overnight before use.

2. Stimulate cell lines with HRG in culture media for 10 minutes at 37° C. Exemplary doses of HRG are 0, 0.032, 0.16, 0.8, 4, 20, 100 nM for MCF-7 cells.

3. Aspirate culture media, transfer onto ice, and add lysis buffer (described below) to lyse cells in situ.

4. Scrape and transfer lysate to microfuge tube. Incubate on ice for 30 min. Microfuge at 14,000 rpm, 4° C., for 10 min.

5. Collect supernatants as lysates and aliquot for storage at −80° C. until use.

Lysis Buffer (Made Fresh and Stored on Ice):

| Final | ul | | Stock |
|---|---|---|---|
| 1% Triton X-100 | 1000 | | 10% |
| 20 mM Tris-HCl (pH 7.5) | 200 | | 1 M |
| 100 mM NaCl | 200 | | 5 M |
| 50 mM NaF | 500 | | 1 M |
| 50 mM Na beta-glycerophosphate | 1000 | | 0.5 M |
| 1 mM Na₃VO₄ | 100 | | 0.1 M |
| 5 mM EDTA | 100 | | 0.5 M |
| 10 ug/ml pepstatin | 100 | | 1 mg/ml |
| 1 tablet (per 10 ml) Roche Complete protease | N/A | | N/A |
| inhibitor (#1836170) | | | |
| Water | 6500 | | N/A |
| | 10 | ml | Total |

The total assay volume is 40 ul. The lysate volume is adjusted to 10 ul with lysis buffer. The antibodies are diluted in lysis buffer up to 20 ul. Typically ˜5000 to 500,000 cell-equivalent of lysates is used per reaction.

Procedure: Working concentrations of pre-mixed antibodies prior to adding into reaction: For Her-3/PI3K complex with cleaving probe at Her-3 (the design of FIG. 9A)

eTag1_anti-BAD at 10 nM

eTag2_anti-Bcl-2 at 10 nM

eTag3_anti-14-3-3 at 10 nM

Biotin_anti-BAD at 20 nM

Universal Standard US-1 at 700 nM

[The Universal Standard US-1 is BSA conjugated with biotin and molecular tag Pro8, which is used to normalize the amount of streptavidin-photosensitizer beads in an assay]. The molecular tags are attached directly to antibodies by reacting an NHS-ester of a molecular tag precursor (see FIGS. 4A-4J) with free amines on the antibodies using conventional techniques, e.g. Hermanson (cited above).

6. To assay 96-well filter plate (Millipore MAGVN2250), add 20 ul antibody mix to 10 ul lysate and incubate for 1 hour at 4° C.

7. Add 10 ul streptavidin-derivatized cleaving probe (final 4 ug/well) to assay well and incubate for 40 min.

8. Add 200 ul wash buffer and apply vacuum to empty.

9. Add 30 ul illumination buffer and illuminate.

10. Transfer 10 ul of each reaction to CE assay plate for analysis.

Data Analysis:

1. Normalize relative fluorescence units (RFU) signal of each molecular tag against that of internal Universal Standard US-1.

2. Subtract RFU of “no lysate” background control from corresponding normalized eTag reporter signals.

The assay readout may be provided as bar graphs as illustrated in FIGS. 9B and 9C. These figures illustrate how the distribution of BAD between 14-3-3 and Bcl-2 would be manifested. In FIGS. 9B and 9C, the leftmost bar for molecular tag 1, “mT₁,” is a measure of the total amount of BAD in the assay, either in complex with 14-3-3, in complex with Bcl-2, or in monomeric form. In FIG. 9B, a situation is illustrated in which relatively more BAD is bound to 14-3-3 than Bcl-2, as indicated by the rightmost bar being larger than the middle bar. In FIG. 9C, the converse situation is illustrated; namely, relatively more BAD is bound to Bcl-2 than 14-3-3, as indicated by the rightmost bar being smaller than the middle bar.

### Example 4

### Correlation Between Her2-Her3 Heterodimer Measurements and Her3-PI3K Intracellular Complex Measurements in Breast Tumor Samnples

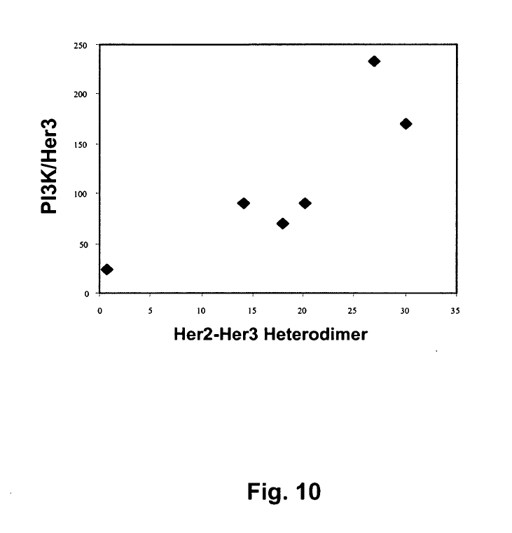

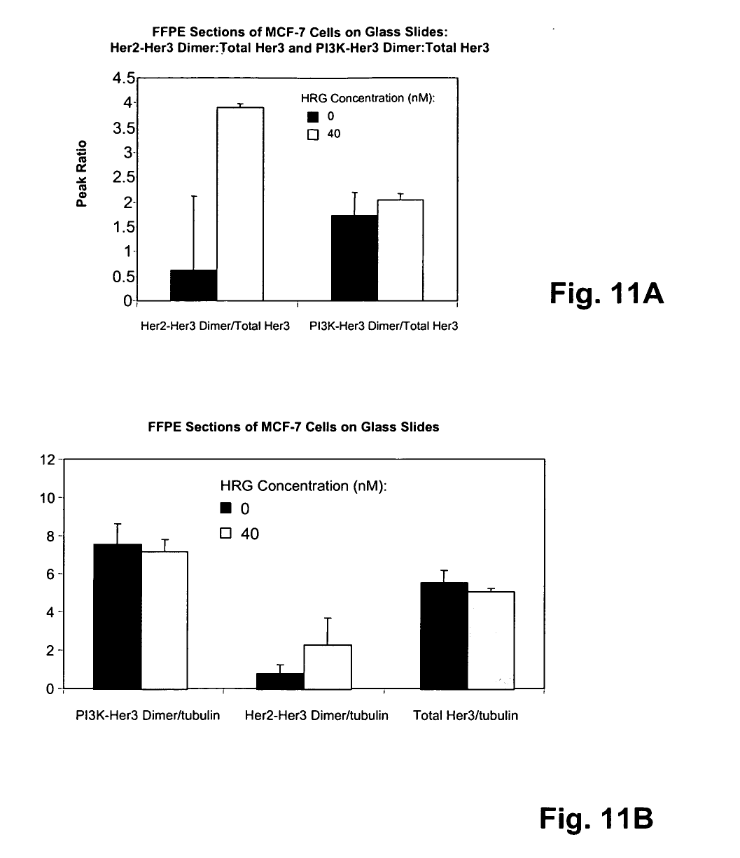

In this example, human breast tumor samples were separately assayed using the methods described above to determine the amounts of Her2-Her3 heterodimers and the amounts of Her3-PI3K complex. FIG. 10 illustrates data obtained from such assays, which shows that the two measurements are correlated.

### Example 5

### Measurement of Intracellular Complex in Formalin Fixed Paraffin Embedded Tissue Samples

In this example, model fixed tissues made from pelleted cell lines were assayed for the presence of PI3K-Her3 complexes. The assay design for heterodimers was essentially the same as that described in FIG. 7A, with exceptions as noted below.

Model fixed tissues were prepared as follows: cells grown on tissue culture plates were stimulated with either EGF or HRG as described in the prior examples, after which they were washed and removed by scrapping. The removed cells were centrifuged to form a pellet, after which formalin was added and the mixture was incubated overnight at 4° C. The fixed pellet was embedded in paraffin using a Miles Tissue Tek III Embedding Center, after which 10 μm tissue sections were sliced from the pellet using a microtome (Leica model 2145). Tissue sections were placed on positively charged glass microscope slides (usually multiple tissue sections per slide) and baked for 1 hr at 60° C.

Tissue sections on the slides were assayed as follows: Tissue sections on a slide were de-waxed with EZ-Dewax reagent (Biogenex, San Ramon, Calif.) using the manufacturer's recommended protocol. Briefly, 500 μL EZ-Dewax was added to each tissue section and the sections were incubated at RT for 5 min, after which the slide was washed with 70% EtOH. This step was repeated and the slide was finally rinsed with deionized water, after which the slide was incubated in water at RT for 20 min. The slide was then immersed into a 1× Antigen Retrieval solution (Biogenesis, Brentwood, NH) at pH 10, after which it was heated for 15 min in a microwave oven (5 min at high power setting followed by 10 min at a low power setting). After cooling to RT (about 45 min), the slide was placed in a water bath for 5 min, then dried. Tissue sections on the dried slide were circled with a hydrophobic wax pen to create regions capable of containing reagents placed on the tissue sections (as illustrated in FIGS. 1F-1G), after which the slide was washed three times in 1× Perm[Wash (BD Biosciences). To each section 50-100 μL blocking buffer was added, and the slide was placed in a covered humidified box containing deionized water for 2 hr at 4° C., after which the blocking buffer was removed from each section by suction. (Blocking buffer is 1× Perm/Wash solution with protease inhibitors (Roche), phosphatase inhibitors (sodium floride, sodium vanadate, β-glycerol phosphate), and 10% mouse serum). To each section 40-50 μL of antibody mix containing binding compounds and cleaving probe was added (each at 5 μg/mL, except that biotin-Ab5 (anti-Her1) was at 10 μg/mL in the Her1-Her2 assay), and the slide was placed in a humidified box overnight at 4° C. The sections were then washed three times with 100 μL Perm/Wash containing protease and phosphatase inhibitors, after which 50 μL of photosensitizer in 1× Perm/Wash solution (containing protease and phosphatase inhibitors) was added. The slide was then incubated for 1-1.5 hr at 4° C. in the dark in a humidified box, after which the photosensitizer was removed by suction while keeping the slide in the dark. While remaining in the dark, the slide was then immersed in 0.01×PBS and incubated on ice for 1 hr. The slide was remove from the PBS, dried, and to each section, 40-50 μL 0.01×PBS with 2 pM fluorescein was added, after which it was illuminated with a high power laser diode (GaAIAs IR emitter, model OD-880W, OPTO DIODE CORP, Newbury Park, Calif.) for 1 hr. The fluorescein acts as a standard to assist in correlating peaks in an electropherogram with moleuclar tags. After illumination, the solution covering each tissue section was mixed by gentle pipeting and transferred to a CE plate for analysis on an Applied Biosystems (Foster City, Calif.) model 3100 capillary electrophoresis instrument.

FIG. 15A shows data from analysis of Her2-Her3 heterodimers and PI3K-Her3 dimers in sections from fixed pellets of MCF-7 cells either non-stimulated or stimulated with 40 nM HRG. The assay design for PI3K-Her3 was essentially as described in FIG. 7A. The above fixation protocol was followed in both cases, except that neither sample was treated with antigen retrieval reagents. The data show that Her2-Her3 dimers increased with treatment by HRG, but that the amount of PI3K-Her3 dimer remained essentially unchanged.

FIG. 15B shows data from analysis of total PI3K, total Her2-Her3 dimer, and total Her3 all relative to amount of tubulin. Tubulin was measured in a conventional sandwich-type assay employing a cleavage probe and a binding compound with a molecular tag. Tubulin was measured to test procedures for normalizing dimer measurement against a target representative of total cell number in a sample, which may be required for measurements on samples with heterogeneous cell types. The data show that the ratios of PI3K-Her3 and Her2-Her3 to tubulin are qualitatively the same as the measurements directly on PI3K-Her3 and Her2-Her3.

What is claimed is:

1. A method of determining disease status of a patient suffering from a disease characterized by aberrant expression of one or more intracellular complexes, the method comprising the steps of:
measuring directly in a patient sample an amount of each of one or more intracellular complexes;
comparing each such amount to its corresponding amount in a reference sample; and
correlating differences in the amounts from the patient sample and the respective corresponding amounts from the reference sample to the disease status the patient.

2. The method of claim 1 wherein said patient sample is a fixed tissue sample, a frozen tissue sample, or circulating epithelial cells.

3. The method of claim 2 wherein said one or more intracellular complexes are selected from the group consisting of 14-3-3//BAD, BID//BAX, BAX//BAX, Bcl-X_{L}//BAD, Bcl-2//BAD, 14-3-3//BID, BID//BAK, BAX//Bcl-2, Bcl-X_{L}//BIK, Bcl-2//BIK, NF-kB//I-kB, BID//Bcl-2, Bcl-X_{L}//BID, Bcl-2//BID, FADD//caspase-9, BID//Bcl-X_{L}, Bcl-X_{L}//Hrk, Bcl-2//Hrk, TRADD//caspase-9, BID//A1/Bfl-1, Bcl-X_{L}//BIM, Bcl-2//BIM, Apaf-1//caspase-9, Bcl-X_{L}//Noxa, Bcl-2//Noxa, Bcl-X_{L}//Bmf, Bcl-2//Bmf, Bcl-X_{L}//Puma, Bcl-2//Puma, Bcl-X_{L}//Bcl-G, Bcl-2//Bcl-G, Bcl-X_{L}//NIP3, Bcl-2//NIP3, Bcl-X_{L}//Nix, and Bcl-2//Nix.

4. The method of claim 2 wherein said one or more intracellular complexes are selected from the group consisting of 14-3-3//BAD, Bcl-2//BAD, 14-3-3//BID, BAX//Bcl-2, Bcl-2//BIK BID//Bcl-2, Bcl-2//BID, Bcl-2//Hrk, Bcl-2//BIM, Bcl-2//Noxa, Bcl-2//Bmf, Bcl-2//Puma, Bcl-2//Bcl-G, Bcl-2//NIP3, and Bcl-2//Nix.

5. The method of claim 4 wherein said disease is a cancer.

6. The method of claim 2 wherein said one or more intracellular complexes is NF-kB//I-kB.

7. The method of claim 6 wherein said disease is an inflammatory condition.

8. The method of claim 2 wherein said one or more intracellular complexes are selected from the group consisting of Her1//Shc, Grb2//Sos, Her1//Grb7, Her1//RasGAP, Grb2//Shc, Her2//Shc, Her3//PI3K, Her3//Shc, Her3//Grb7, YAP//Her4, IGF-1R//PI3K, IGF-1R//Shc, IGFR//IRS1, VEGFR//Shc, VEGFR//PI3K, VEGFR//Src, VEGFR//FRS2, PDGFRa//Crk, PDGFR//Grb2, PDGFR//Grb7, PDGFR//Nck; PDGFR//Shc, DGFR//STAT5, PDGFRa//Crk, PDGFRb//GAP, PDGFR//Grb2, PDGFR//Grb7, PDGFR//Nck; PDGFR//Shc, PDGFR//Shp2, PDGFR//RasGAP, PDGFR//STAT5, PDGFRb//GAP, PDGFR//Grb2, PDGFR//Grb7, PDGFR//Nck, PDGFR//Shc, PDGFR//Shp2, PDGFR//RasGAP, PDGFR//STAT5, Kit//Shp-1, Kit//PI3K, Kit//Grb2, Kit//CRKL, FGFR//PLCg1, FGFR//Crk, FGFR//FRS2, GFR//Shp2, FGFR//Shb, Trk//p75NTR, and Trk//PI3K.

9. The method of claim 8 wherein said disease is a cancer.

10. The method of claim 2 wherein said one or more intracellular complexes are selected from the group consisting of Her1//Shc, Grb2//Sos, Her1//Grb7, Her1//RasGAP, Grb2//Shc, Her2//Shc, Her3//PI3K, Her3//Shc, and Her3//Grb7.

11. The method of claim 10 wherein said disease is a cancer.

12. The method of claim 11 wherein said cancer is breast cancer, ovarian cancer, colorectal cancer, or prostate cancer.

13. The method of claim 2 wherein said disease is a cancer and wherein said one or more intracellular complexes are selected from the group consisting of IGF-1R//PI3K, IGF-1R//Shc, and IGFR//IRS1.

14. The method of claim 2 wherein said disease is a cancer and wherein said one or more intracellular complexes are selected from the group consisting of VEGFR//Shc, VEGFR//PI3K, VEGFR//Src, and VEGFR//FRS2.

15. The method of claim 2 wherein said disease is a cancer and wherein said one or more intracellular complexes are selected from the group consisting of PDGFRa//Crk, PDGFR//Grb2, PDGFR//Grb7, PDGFR//Nck; PDGFR//Shc, PDGFR//STAT5, PDGFRa//Crk, PDGFRb//GAP, PDGFR//Grb2, PDGFR//Grb7, PDGFR//Nck; PDGFR//Shc, PDGFR//Shp2, PDGFR//RasGAP, PDGFR//STAT5, PDGFRb//GAP, PDGFR//Grb2, PDGFR//Grb7, PDGFR//Nck, PDGFR//Shc, PDGFR//Shp2, PDGFR//RasGAP, and PDGFR//STAT5.

16. The method according to claim 1, 2, 3, 4, 5, 6, 7, 8, 9, 10, 11, 12, 13, 14, or 15 wherein each of said one or more intracellular complexes are determined by the steps of:
providing for each of said one or more intracellular complexes a reagent pair comprising a cleaving probe having a cleavage-inducing moiety with an effective proximity, and one or more binding compounds each having one or more molecular tags attached thereto by a cleavable linkage, the molecular tags of different binding compounds having different separation characteristics;
mixing the cleaving probe and the one or more binding compounds for each of said one or more intracellular complexes with said patient sample such that the cleaving probe and the one or more binding compounds specifically bind to their respective intracellular complexes and the cleavable linkages of the one or more binding compounds are within the effective proximity of the cleavage-inducing moiety so that molecular tags are released; and
separating and identifying the released molecular tags to determine the presence or absence or the amount of said one or more intracellular complexes in said patient sample.

17. A method of determining an apoptotic status of cells in a sample, the method comprising the step of simultaneously measuring amounts of at least one complex selected from the group consisting of a complex comprising a Bcl-2 protein and a BH3-only protein and a complex comprising a 14-3-3 protein and a BAD protein.

18. The method of claim 17 further including simultaneously measuring free NF-KB proteins and free I-kB proteins, and wherein said group further consist of a complex comprising an NF-kB protein and an I-kB protein.

19. The method of claim 18 wherein said group further consists of a homodimer comprising a BAX protein.

20. The method according to claim 17, 18, or 19 wherein each of said one or more intracellular complexes are determined by the steps of:
providing for each of said one or more intracellular complexes a reagent pair comprising a cleaving probe having a cleavage-inducing moiety with an effective proximity, and one or more binding compounds each having one or more molecular tags attached thereto by a cleavable linkage, the molecular tags of different binding compounds having different separation characteristics;
mixing the cleaving probe and the one or more binding compounds for each of said one or more intracellular complexes with said patient sample such that the cleaving probe and the one or more binding compounds specifically bind to their respective intracellular complexes and the cleavable linkages of the one or more binding compounds are within the effective proximity of the cleavage-inducing moiety so that molecular tags are released; and
separating and identifying the released molecular tags to determine the presence or absence or the amount of said one or more intracellular complexes in said patient sample.

21. A method of determining a status of a cancer in a patient, the method comprising the step of simultaneously measuring in a sample from the patient amounts of at least one intracellular complex selected from the group consisting of a first complex comprising a Bcl-2 protein and a BH3-only protein and a second complex comprising a 14-3-3 protein and a BAD protein.

22. The method of claim 21 wherein said at least one intracellular complexes is determined by the steps of:
providing for each of said first and second intracellular complexes a reagent pair comprising a cleaving probe having a cleavage-inducing moiety with an effective proximity, and one or more binding compounds each having one or more molecular tags attached thereto by a cleavable linkage, the molecular tags of different binding compounds having different separation characteristics;
mixing the cleaving probe and the one or more binding compounds for each of said first and second intracellular complexes with said patient sample such that the cleaving probe and the one or more binding compounds specifically bind to their respective intracellular complexes and the cleavable linkages of the one or more binding compounds are within the effective proximity of the cleavage-inducing moiety so that molecular tags are released; and
separating and identifying the released molecular tags to determine the presence or absence or the amount of said first and second intracellular complexes in said patient sample.

23. A method of determining an apoptotic status of cells in a sample, the method comprising the step of simultaneously measuring amounts of at least one complex selected from the group consisting of a complex comprising an NF-kB protein and an I-kB protein and amounts of free NF-KB proteins and free I-kB proteins.
